(19) United States
(12) Patent Application Publication
(10) Pub. No.: US 2026/0256926 A1
(43) Pub. Date: Sep. 3, 2026

(54) BIFACIAL PEPTIDE NUCLEIC ACID PROBES AND METHODS OF USING THEREOF

(71) Applicant: Ohio State Innovation Foundation, Columbus, OH (US)

(72) Inventors: Dennis BONG, Columbus, OH (US); Shiqin MIAO, Gaithersburg, MD (US); Yufeng LIANG, Gaithersburg, MD (US)

(21) Appl. No.: 18/992,112

(22) PCT Filed: Jul. 10, 2023
(86) PCT No.: PCT/US2023/027292

§ 371 Date: Jan. 7, 2025

Related U.S. Application Data
(60) Provisional application No. 63/359,500, filed on Jul. 8, 2022.

(51) Int. Cl. A61K 47/55; A61K 47/62; A61K 49/00; C12Q 1/6825

(52) U.S. Cl. CPC A61K 47/55; A61K 47/62; A61K 49/0056; C12Q 1/6825

(57) ABSTRACT

Disclosed herein are bifacial peptide nucleic acid probes. These probes can include a triplex hybrid forming moiety (e.g., a plurality of binding motifs disposed along a peptidyl backbone) conjugated to a prosthetic group. The triplex hybrid forming moiety can bind to a non-canonical base pairing site in a target nucleic acid via triplex hybridization. In this way, a variety of prosthetic groups (e.g., dyes, labels, reactive groups, etc.) can be selectively delivered to a target nucleic acid (e.g., in vivo, in vitro, or ex vivo).

### CROSS-REFERENCE TO RELATED APPLICATIONS

This application claims benefit of U.S. Provisional Application No. 63/359,500, filed Jul. 8, 2022, which is hereby incorporated herein by reference in its entirety.

### STATEMENT REGARDING FEDERALLY SPONSORED RESEARCH OR DEVELOPMENT

This invention was made with government support under Grant No. R01 GM143543 and R01 GM111995 awarded by the National Institutes of Health. The government has certain rights in the invention.

### BACKGROUND

There remains a need for improved and orthogonal methods for localizing and tracking intracellular RNA and DNA molecules in real-time. Despite the existence of many useful RNA imaging strategies, there are limitations on placement of probe binding sites and concerns regarding overall structural impact of labeling on the RNA of interest. Modification of internal sites in RNA via chemical reaction or duplex hybridization can be technically challenging or sequence-limited. While fluorescence in situ (duplex) hybridization (FISH) with DNA or PNA probes is broadly used for labeling RNAs, FISH is limited to fixed cell experiments. Molecular beacons may be used with unmodified cells and transcripts in live cell labeling but require electroporation or microinjection strategies and have not been as widely adopted as other methods. In addition, duplex hybridization to internal sites destroys existing secondary structures via strand invasion or transforms a single-stranded loop into a new duplex stem where none existed previously, introducing potentially disruptive steric interactions.

Beyond chemical modification and FISH, approaches to intracellular RNA tracking may be roughly divided into small molecule dye binding and protein labels. Dye-binding SELEX-derived aptamers (eg—Spinach, Broccoli, Corn, Mango, Pepper) have been inserted into RNAs of interest for tracking and sensing applications. A variation on this approach exploits RNA binding to dequench dye-quencher conjugates (eg-SRB2, Gemini-561, Riboglow) and can utilize native aptamers to universal quenchers such as cobalamin; this method may be applied to a wider range of fluorophores without the need for additional aptamer selection. However, all aptamer methods all require insertion of a distinct folded structure within the ROI that has the potential to alter native RNA sterics or lifetime. Further, early fluorescent aptamer designs suffered from insufficient brightness thought to be due to misfolded RNA loops. In particular, the G-quadruplex motif common to Mango and Spinach aptamers is especially labile to intracellular degradation and requires non-native levels of potassium and magnesium, which may result in deficient intracellular aptamer performance.

Similarly, bacteriophage-derived RNA hairpins MS2 and PP7 are native sequences that bind to phage coat proteins MCP (MS2 coat protein) and PCP (PP7 coat protein), respectively. The most widely used RNA tracking methodology is to insert MS2 into ROIs and image with MCP fused to fluorescent proteins (MCP-FPs) or MCP-HaloTag and MCP-SNAP tag fusions, with subsequent dye staining of the MS2 RNPs. Though effective, the use of constitutively fluorescent dyes and FPs generates significant background signal, requiring multiple (often >20) MS2 hairpins to generate sufficient signal to noise. Further, these protein labeling systems carry significant steric bulk (>40 kD for a single MCP-FP) with the risk of altered transcript decay and inhibited native contacts. Despite these drawbacks, MS2/PP7 labeling remains the most widely used method for RNA tracking. The use of CRISPR-Cas/guide RNA complexes for targeting may be used with or without RNA modification, but similarly requires the formation of sterically encumbering synthetic RNPs with native RNA targets or genomic loci.

Accordingly, there remains a need for improved methods for localizing and tracking intracellular RNA and DNA molecules in real-time.

### SUMMARY

Provided herein are bifacial peptide nucleic acid probes. These probes can include a triplex hybrid forming moiety (e.g., a plurality of binding motifs disposed along a peptidyl backbone) conjugated to a prosthetic group. The triplex hybrid forming moiety can bind to a non-canonical base pairing site in a target nucleic acid via triplex hybridization. In this way, a variety of prosthetic groups (e.g., dyes, labels, reactive groups, etc.) can be selectively delivered to a target nucleic acid (e.g., in vivo, in vitro, or ex vivo).

For example, provided herein are bifacial peptide nucleic acid probes defined by Formula I below

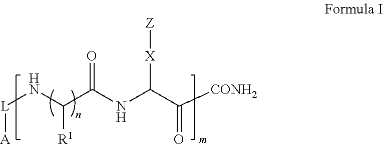

wherein
A represents a prosthetic group;
X is absent or represents a first bivalent linking group;
L is absent or represents a second bivalent linking group;
n is, individually for each occurrence, an integer selected from 1 and 2;
m is an integer selected from 2, 3, and 4;
Z represents, individually for each occurrence, a binding motif selected from one of the following

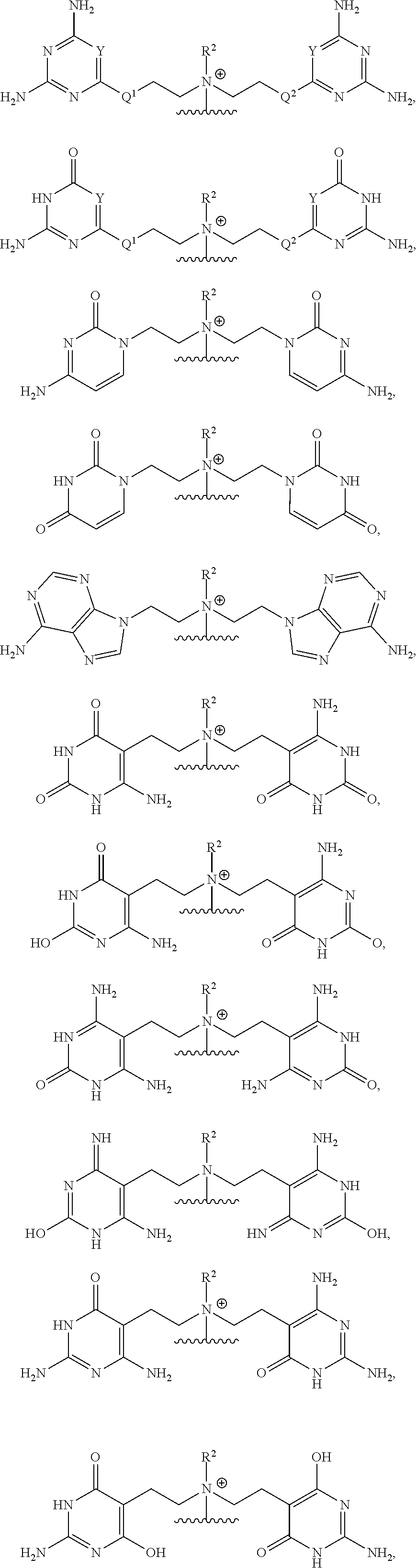

Q¹ and Q² individually represent —O— or —NR^{A}—;
Y is N, —CR^{B}—,
R¹ is, individually for each occurrence, selected from one of the following

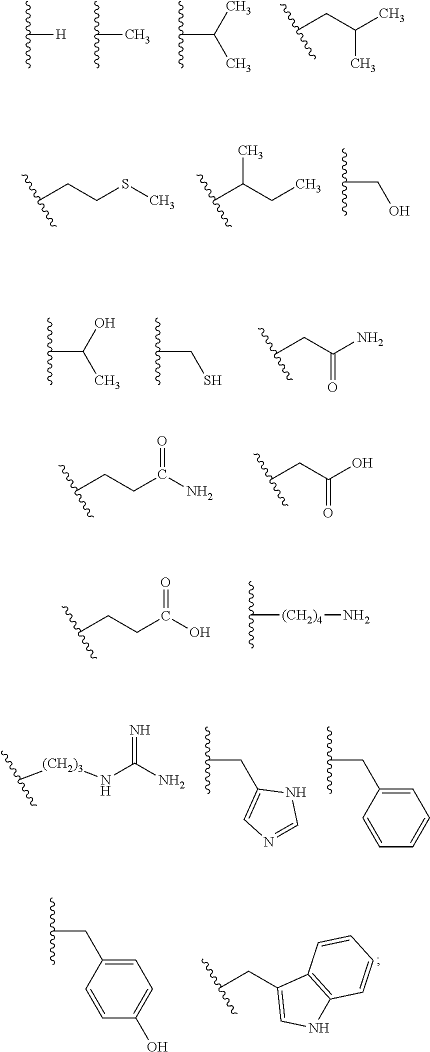

R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In some embodiments, R^{A} represents H for each occurrence (e.g., Q¹ and Q² individually represent —O— or —NH—). In some embodiments, Q¹ and Q² represent —O— for each occurrence. In other embodiments, Q¹ and Q² represent —NH— for each occurrence.

In some embodiments, R² represents H for each occurrence.

In some embodiments, R¹ is, individually for each occurrence, selected from one of the following

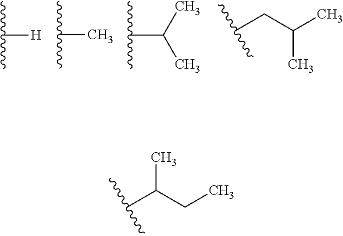

In certain embodiments, R¹ is, individually for each occurrence, —H or —CH₃.

In some embodiments, m is 2. In other embodiments, m is 3.

In some embodiments, n is 1 in all occurrences. In other embodiments, m is 3 and n is 1 in two occurrences and n is 2 in one occurrence.

In some embodiments, X represents a first bivalent linking group. In some embodiments, the first bivalent linking group comprises from 3 to 20 atoms, such as from 3 to 16 atoms or from 3 to 12 atoms. In certain embodiments, the first bivalent linking group comprises an alkylene linker or a heteroalkylene linker.

In some embodiments, L represents a second bivalent linking group. In some embodiments, the second linking group comprises from 3 to 36 atoms, such as from 3 to 24 atoms or from 3 to 16 atoms. In certain embodiments, the second bivalent linking group comprises an alkylene linker or a heteroalkylene linker.

In some embodiments, in at least one occurrence, Z represents the binding motif shown below

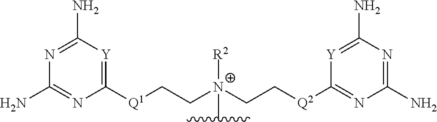

wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In certain embodiments, m is 2 and Z represents the binding motif shown below in two occurrences

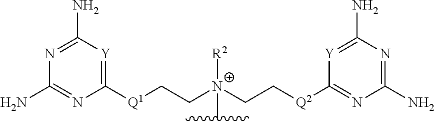

wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and R^{B} represents, individually for each occurrence, H, halogen, methyl, or trifluoromethyl.

In certain embodiments, m is 3 and Z represents the binding motif shown below in two occurrences

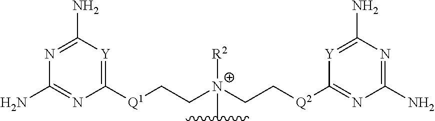

wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In certain examples, the compound is defined by Formula IA below

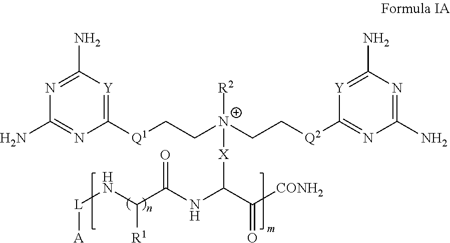

wherein A, L, Y, Q¹, Q², X, n, and m are as defined above

In certain examples, the compound is defined by Formula IB

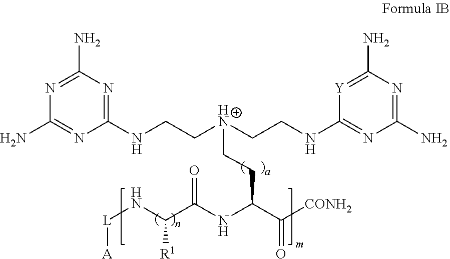

wherein
A, L, R¹, m, and n are as defined above; and
a is, individually for each occurrence, an integer selected from 3, 4, 5, and 6.

In some embodiments, the prosthetic group is not cyanine 5 (Cy5), cyanine 3 (Cy3), or carboxyfluorescein (Cbf).

In some embodiments, the prosthetic group is selected from the group consisting of a fluorogenic dye, a protein ligand, a redox-active center, an ROS-generating center, a photoreactive center, a spin label, a group transfer agent, a therapeutic agent, a catalytic center, a click motif, or an NMR active label.

In some embodiments, the prosthetic group is a fluorogenic dye. In certain embodiments, the fluorogenic dye is selected from the group consisting of thiazole derived dyes such as thiazole orange, dimethylindole red and derivatives thereof, symmetric cyanine dyes, asymmetric cyanine dyes, fluoresceins, rhodamines, fluorogenic variants of asymmetric cyanine dyes and fluoresceins such as JF635 and JF646, arsenate dyes such as FLASH and ReASH, malachite green and derivatives thereof, coumarin dyes, and hydroxybenzylidene dyes. In certain embodiments, the fluorogenic dye is thiazole orange.

In some embodiments, the prosthetic group is a protein ligand. In certain embodiments, the protein ligand is selected from the group consisting of a ubiquitin ligase ligand, a ligand for a translational activator, a ligand for a translational inhibitor, a ligand for a transcription activator, a ligand for a transcription inhibitor, a ligand for a nuclease, or a ligand for a cell surface protein. In certain embodiments, the protein ligand is a ubiquitin ligase ligand that binds to an E3 ligase selected from the group consisting of XIAP, VHL, cereblon, and MDM2.

In some embodiments, the compounds described herein can bind to a non-canonical base pairing site in a nucleic acid (e.g., RNA) via triplex hybridization. In certain embodiments, the non-canonical base pairing site comprises a U-rich internal loop (URIL). In certain embodiments, the URIL is a loop that includes at least four non-canonical base pairs, wherein at least 50% of the non-canonical base pairs comprise U—U pairs. In certain embodiments, the URIL comprises from 4-8 non-canonical base pairs.

Also provided are methods of detecting a target nucleic acid. These methods can comprise contacting the target nucleic acid with a bifacial peptide nucleic acid probe comprising a triplex hybrid forming moiety conjugated to a fluorogenic dye, wherein the bifacial peptide nucleic acid probe binds to a non-canonical base pairing site in the target nucleic acid via triplex hybridization.

Also provided are methods of selectively degrading a target protein in vivo. These methods can comprise contacting a nucleic acid with a bifacial peptide nucleic acid probe comprising a triplex hybrid forming moiety conjugated to a ubiquitin ligase ligand, wherein the bifacial peptide nucleic acid probe binds to a non-canonical base pairing site in the target nucleic acid via triplex hybridization; wherein the nucleic acid forms a ribonucleoprotein complex with the nucleic acid; and wherein the ubiquitin ligase ligand of the bifacial peptide nucleic acid probe bound to the nucleic acid directs ubiquitination of the target protein, thereby directing proteolysis of the target protein.

Also provided are methods of selectively labeling a target protein in vivo. These method can comprise contacting a nucleic acid with a bifacial peptide nucleic acid probe comprising a triplex hybrid forming moiety conjugated to a redox-active center, an ROS-generating center, a photoreactive center, or a catalytic center, wherein the bifacial peptide nucleic acid probe binds to a non-canonical base pairing site in the target nucleic acid via triplex hybridization; wherein the nucleic acid forms a ribonucleoprotein complex with the target protein; and wherein the redox-active center, the ROS-generating center, the photoreactive center, or the catalytic center of the bifacial peptide nucleic acid probe bound to the nucleic acid participates in a chemical reaction that results in covalent modification of the target protein, thereby labeling the target protein.

In some embodiments, the bifacial peptide nucleic acid probe comprises a triplex hybrid forming moiety conjugated to an ROS-generating center.

In some embodiments, the nucleic acid forms a ribonucleoprotein complex with the target protein; wherein the ROS-generating center generates radical species that react with biotin-phenol to generate a phenoxy radical which reacts with the target protein, thereby labeling the target protein with biotin.

### DESCRIPTION OF DRAWINGS

FIGS. 1A-1B illustrate FLURIL-tagging of RNAs with bPNA probes. FIG. 1A shows triplex hybridization of a U-rich internal loop (URIL) with bPNA (blue) via base triple formation between the melamine base (M) and two uracil bases (inset). FIG. 1B shows a general schematic of labeling strategy described herein. An RNA of interest is engineered to contain an URIL and expressed within the cell, with a fluorogenic bPNA probe introduced via cell culture media. Successful URIL targeting is reported by an increase in emission (green) and confirmed by a previously established RNA binding protein with a fluorescent protein (red) fusion.

FIG. 2 illustrates example synthetic bPNA probes. Structures of fluorogenic bPNA probes studied, with thiazole orange (TO) dye and K^{2M} sidechain structure shown inset. Lower case k^{2M} residue denotes D-configuration and K^{TO} indicates sidechain is acylated with R₁ (TO). For TO-K^{2M}AK^{2M} and TO-K^{2M}IK2^{M}, R₁ includes a β-alanine spacer between TO and bPNA.

FIGS. 3A-3D illustrate the design of URIL RNA constructs for bPNA probe binding. FIG. 3A shows RNAs for in vitro evaluation were prepared via run-off transcription or purchased directly. The 12-U6-12 and 12-U4-12 constructs feature 12 bp duplexes (indicated by dashed lines) separated by U₆ₓU₆ and U₄ₓU₄ internal bulges, respectively. FIG. 3B shows RNA constructs for fixed cell labeling (HEK-293T) of RNPs are shown; hairpin RNAs were used to replace the anticodon stem of tRNA^{Lys} (dashed lines) or appended to a TDP-43 binding (GU) s sequence (SEQ ID NO. 53). Constructs for live cell (U2OS) RNA tracking with (FIG. 3C) FLURIL-tagging and (FIG. 3D) Sirius IDR2-(MS2)₈ with MS2 (MBSV5)⁶¹ hairpins shown in red. IDR3-U4 carries a single PP7 hairpin as an internal control. RNAs for cell experiments were delivered by plasmid transfection.

FIGS. 4A-4D show the characterization of fluorogenic bPNA hybridization with URIL RNAs. FIG. 4A shows the normalized absorbance and emission of a representative TO-bPNA 1 hybrid with RNA. FIG. 4B shows the relative fluorescence units (RFU) of fluorescein standard and the hybrid in (A) indicating relative brightness (Φ=43%, ε₅₀₇=44,883 M⁻¹ cm⁻¹). In vitro fluorescence of (FIG. 4C) bPNAs alone or with RNAII (lacking a URIL) and (FIG. 4D) upon treatment with RNA constructs that have a URIL binding site (FIG. 2). The mean value of 3 separate measurements with standard deviation error for all RNA and bPNA combinations are shown under identical conditions (50 mM HEPES, pH 7.5, 100 mM NaCl, 1 μM bPNA and RNA.

FIGS. 5A-5B show the URIL-specific intracellular fluorescence. FIG. 5A shows the integrated fluorescence of U4-tRNA, NEG-tRNA and untreated cells, normalized to untreated cells. The mean value of 10 independent cell measurements of normalized fluorescence (scatter plot) and standard deviation error is shown. All cell samples were treated with TO-bPNA 1. FIG. 5B shows representative confocal fluorescence microscopy images from triplicate studies of HEK-293T cells treated with 1 in media and transfection with plasmid encoding (top row) U4 tRNA and (bottom row) NEG tRNA. 170/177 (97%) of cells were labeled with TO-bPNA. Scale bar=50 μm.

FIG. 6 illustrates simultaneous MS2 and FLURIL imaging of RNPs using MCP-RFP and TO-bPNA. Representative confocal fluorescence microscopy images of HEK-293T cells treated as indicated at left of each row and imaged under the dye channels indicated at the top of each column. Experiments were performed in triplicate. Scale bar=10 μm. The MCP-RFP fusion also contains a nuclear localization tag. The first row is imaged two hours after transfection and treatment with TO-K^{2M}Ala-K^{2M} (1 μM) in media while subsequent rows are imaged 8 hours after transfection.

FIG. 7 shows the imaging native RNP pairs using TDP-43-tdTomato and TO-bPNA. Representative confocal fluorescence microscopy images of HEK-293 cells treated as indicated at left of each row and imaged under the dye channels indicated at the top of each column. Experiments were performed in triplicate. Scale bar=10 μm. Top row indicates nuclear colocalization of TDP-43-tdTomato with TO-K^{2M}Ala-K^{2M} fluorescence when co-expressed with U4-(GU) s. Lower two rows feature mismatches between RNA and RBP (U4-(GU)₈ with MCP-RFP and U4-MS2 with TDP-43-tdTomato, respectively), resulting in partitioning of TO-K^{2M}Ala-K^{2M} emission to the cytoplasm.

FIGS. 8A-8B show FLURIL-tags in CRISPR-dCas live cell genomic loci tracking. FIG. 8A is an illustration of dual color genomic labeling of IDR2 and IDR3 by CRISPR-dCas9 targeting. IDR3 is tracked by FLURIL-tagging of IDR3-targeting gRNA while MS2-labeling of IDR2-targeting gRNA is used to track IDR2. FLURIL-tags are stained with TO-bPNA while MS2 labels are stained with MCP-HaloTag binding and HaloTag-JF549 reaction with the complex. MS2 labeling is accomplished using the CRISPR-Sirius gRNA design in Sirius-IDR2-(MS2)₈ gRNA which has an array of 8 MS2 hairpins. FIG. 8B shows the plasmids used: (Top) dual-gRNA plasmid driven by two promoters (hU6 for Sirius-IDR2-(MS2)₈, CMV for IDR3-U4), (Middle) control dual gRNA plasmid with IDR3-U4 gRNA replaced by Sirius-IDR3-(PP7)₈, (Bottom) plasmids carrying dCas9 under an inducible promoter, MCP-Halotag fusion under continuous expression. NLS=nuclear localization signal; P2A=cleavage peptide; HSA=mouse heat stable antigen.

FIG. 9 shows dual-color CRISPR imaging of IDR2 and IDR3 genomic loci in U2OS by simultaneous FLURIL-tagging and MS2-labeling of gRNAs. (Top Row) Representative loci imaging from triplicate independent measurements, following transfection with dual plasmid encoding Sirius-IDR2-(MS2)₈ and IDR3-U4 followed by staining with Halo-JF549 (red) and TO-bPNA (green), respectively. Genomic loci highlighted in the white square are shown enlarged, inset bottom right-hand corner. (Middle Row) Same plasmid transfection as top row without TO-bPNA treatment. (Bottom Row) Loci imaging following transfection with dual plasmid encoding Sirius-IDR2-(MS2)₈ and Sirius-IDR3-(PP7): followed by staining with Halo-JF549 (red) and TO-bPNA (green), respectively. Bar in the inset, 1 μm.

FIG. 10 shows FLURIL-tag Tracking of IDR2 and IDR3 dynamics in live cells. U2OS cells were imaged 48 hours post-transfection with dual plasmid encoding Sirius-IDR2-(MS2) s and IDR3-U4 and stained with HaloTag-JF549 (red) and TO-bPNA (green) Representative loci trajectories of 96 frames from homologous chromosomes are shown in the top and bottom from triplicate independent measurements. The imaging rate is 136 ms per frame. Bar in the inset, 1 μm. For comparison, trajectories were aligned to start from the origin (0,0).

FIG. 11 illustrates the predicted RNA secondary structures for URIL RNA probe constructs. (SEQ ID NO. 48, top left, NEG tRNA; SEQ ID NO. 7, top right, U4-tRNA; SEQ ID NO. 49, bottom left, MS2-U4 tRNA; SEQ ID NO. 10, bottom right, U4-(GU)₈))

FIG. 12 shows the sequence and structure of IDR3-U4 single gRNAs. CRISPRainbow with two PP7 hairpins (2×PP7) gRNA was modified to carry the U 4×U 4 internal bulge (URIL) at the second loop position. The cyan color shows crRNA that targets the endogenous DNA sequences (purple). PAM sequence is shown in orange. Red indicates the gRNA scaffold and PP7 and URIL hairpin scaffolds are shown in green. The PP7 hairpin was carried over from a prior sgRNA construct that was modified and serves as an internal control for labeling experiments that lack bPNA probe. (SEQ ID NO. 50, SEQ ID NO. 51, SEQ ID NO. 52, shown from top to bottom)

FIG. 13 shows high focus-to-nuclear background ratio of 1×URIL-TO-bPNA for DNA imaging. Comparison of the focus-to-nuclear background ratio using CRISPR-Sirius-IDR3-8×PP7/PCP-GFP (sgRNA structure as shown in FIG. 2D) and IDR3-1×URIL-TO-bPNA (sgRNA structure as shown in FIG. 3C and FIG. 11). The nuclear periphery is outlined in pink. Insets show enlarged images of the genomic loci. (Top row) Images were captured using the same microscope settings and scaled to the same grey levels. All experiments were repeated at least three times (biological replicates). (Bottom) Quantification of the focus/nucleoplasm intensity ratio for the two labeling methods, respectively. The lines within the boxes represent the mean; the outer edges of the box are the 10 th and 90 th percentiles; the whiskers extend to the minimum and maximum values; n cell=31 for each experiment. The significance test (two-tailed Welch t-test) was performed at 95% confidence level.

FIG. 14 shows the cellular fluorescence intensity following treatment with Cy5-bPNA in place of TO-bPNA. HEK-293 cells were transfected as previously described with U4-tRNA, negative-tRNA or no tRNA. The relative intensities were normalized. Data from 10 independent measurements are shown, with mean values indicated by the top of the bar and standard deviation error shown.

FIG. 15 is a plot showing the fluorescence activation (apparent K_{d}) of TO-K^{2M}-Ala-K^{2M} with 12-U4-12 RNA. Concentration of bPNA is 100 nM. The mean value of 3 independent replicates was plotted with standard deviation shown in error bars.

FIG. 16 shows the structures of the fluorogenic probes used in Example 2. All probes made based on literature procedures to install the R₁ and R² linkers indicated. Ammeline was synthesized with both R₁ and R² linkers to produce N1 and N2, respectively.

FIG. 17 is a heat map of fluorogenic binding for the abasic (Left) DNA and (Right) RNA) libraries. Fluorescence is reported as average intensity from triplicate measurement relative to unbound probe. Samples were prepared in HEPES buffer (pH 7.5, 50 mM, 100 mM NaCl) with 2 μM probe, 2 μM duplex.

FIG. 18 (Top) shows fluorescence heat maps of NCP-probe binding. Average emission intensities from triplicate measurement are shown according to the scales at right, indicating fold enhancement over unbound probes for DNA (Left) and RNA (Right). Probes and NCPs in both maps are ordered with the highest integrated relative fluorescence signal in DNA at the top and right, respectively. (Bottom) shows possible interactions of synthetic bases melamine (M), ammeline (N) and W1 with NCPs approached from the (above) major groove and (below) minor groove. Samples were prepared in HEPES buffer (pH 7.5, 50 mM, 100 mM NaCl) with 2 μM probe, 2 μM duplex.

FIG. 19 (Top) shows a heat map of tandem NCP RNA library screen. Tandem NCPs are ordered by calculated relative stability, with the most stable on the left. Probes are ordered with the highest integrated fluorescence (most reactive) in the top row and lowest in the bottom row. Fluorescence indicated is relative enhancement over background TO emission. (Bottom) shows probe emission data grouped by XQ/WZ size ratio in the 2×2 internal bulge. Size ratio is calculated using purine or pyrimidine molecular weight as an approximation for each base in the tandem NCP. Schematic illustration of the size ratio is indicated above the green bars highlighting the most reactive probe in each grouping. The most reactive probes in each group are labeled accordingly. Samples were prepared in HEPES buffer (pH 7.5, 50 mM, 100 mM NaCl) with 4 μM probe, 2 μM RNA duplex,

FIG. 20 shows binding isotherms of M, W1 and N2 probes binding to the indicated tandem NCPs, obtained by fluorescence (RFU) as a function of RNA (duplex) concentration, showing fit to a 1:1 binding model and standard deviation error bars from triplicate measurements. RNA substrates and probes were the same as in FIG. 19.

FIG. 21 is an illustration of the three nucleic acid library designs targeted by the fluorogenic probe library. (Left) The abasic library in DNA and RNA pairs a purine (R) or pyrimidine (Y) with an abasic site (Ap) wherein the anomeric carbon is replaced with CH₂; the single noncanonical pair (NCP) library contains all non-WC pairings in DNA and RNA; the tandem NCP library in RNA only contains 43 unique motifs of two non-WC pairs in tandem. (Right) Illustration of fluorogenic probe binding to the noncanonical sites. (SEQ ID NO. 54 first strand from left, abasic; SEQ ID NO. 55, second strand from left, abasic; SEQ ID NO. 54, third strand from left, single NCP; SEQ ID NO. 55, fourth strand from left, single NCP; SEQ ID NO. 56, fifth strand from left, tandem NCP; SEQ ID NO. 57, sixth strand from left, tandem NCP)

FIG. 22 illustrates bPNA binding to an RNA URIL with variation in the central tetrad NNxNN).

FIG. 23 shows a heat map of RNA binding gel data, normalized against 6 M bPNA binding to U6xU6. While melamine remains a strong binder, when the central bPNA residue is KNN (left axis) one observes selective binding to a UUxGG central tetrad site in the RNA duplex. Additional hits are seen as well, suggestive of other binding solutions.

FIG. 24 shows (Inset) Spinach binding DFHBI dye (green). (Right) P2 stem replacement with a 6×6 URIL with variation of the central tetrad. Fluorogenic bPNA binding depends on both the central tetrad sequence and the central residue of bPNA (K^{2M}K^{XX}K^{2M}).

FIG. 25 shows potential base triples formed with melamine (M), ammeline (N) and synthetic pyrimidine W1.

FIG. 26 shows an illustration of RNA motif centered PROTACs. Bifacial peptide nucleic acids (bPNAs) bind to U-rich internal loops (URILs) in RNA via base-triple formation between melamine and uracil and present an E3-ligase ligand. Following RNP formation, the E3-ligase ligand recruits an E3-ligase, resulting in ubiquitinylation of the RNP and subsequent degradation by the proteasome.

FIG. 27A shows the general design of the RNA-PROTACs reagent. An E3-ligase ligand is coupled via a linker to bPNA. Examples of established linker and ligands are shown below, which target two distinct E3-ligases, Cereblon/CNBR and VHL. FIG. 27B shows the structure of bPNA used in this example, with triplex hybridization to a URIL below.

FIG. 28 shows tRNA constructs introduced into HEK293 cells for proximity labeling. The MS2 hairpin binds MCP, which is fused to tagRFP, a nuclear-localized red fluorescent protein. (SEQ ID NO. 58, left strand (MS2-U4); SEQ ID NO. 59 middle strand (u4); SEQ ID NO. 60, right strand (neg ctrl))

FIGS. 29A and 29B show confocal fluorescence microscopy images of fixed and permeabalized HEK293 cells. Staining with Hoechst (nuclear) and red fluorescence from RFP. FIG. 29 shows imaging after 24 hr (FIG. 29A) and (FIG. 29B) 72 hr. Experiment column (correctly matched RNA and protein, with POM-bPNA) is the far right of each panel.

FIG. 30. (Top) bPNAs form triplex hybrids with URILs. (Middle) ROS-bPNAs. (Bottom) R₁=ROS-generating metal complexes.

FIG. 31 is an illustration of proof-of-concept proximity labeling experiment. A URIL was juxtaposed with an MS2 hairpin in an RNA construct introduced into HEK-293 cells via plasmid transfection. The cells were co-transfected with a plasmid encoding MCP-RFP fusion protein. An iron-EDTA bearing bPNA is introduced via cell media and allowed to bind and generate ROS in the endogenous reducing environment. The cells are fixed and permeabalized, then stained with Streptavidin-Alexa-488. Confocal microscopy is used to observe co-localization of RFP and Alexa-488 as an indication of proximity labeling.

FIG. 32 shows tRNA constructs introduced into HEK293 cells for proximity labeling. The MS2 hairpin binds MCP, which is fused to tagRFP, a nuclear-localized red fluorescent protein. (SEQ ID NO. 58, left strand (MS2-U4); SEQ ID NO. 59 middle strand (u4); SEQ ID NO. 60, right strand (neg ctrl))

FIGS. 33A and 33B show confocal fluorescence microscopy images of fixed and permeabalized HEK293 cells. Staining with Hoechst (nuclear) and streptavidin-488 (green, anti biotin) and red fluorescence from RFP. (FIG. 33A) Transfection with MS2-U4 RNA and bPNA or biotin-phenol is excluded, resulting in a lack of green stain. (FIG. 33B) Transfection with NEG RNA (fully base-paired, no URIL) and including bPNA and biotin-phenol. No green fluorescence observed.

FIG. 34 shows confocal fluorescence microscopy images of fixed and permeabalized HEK293 cells. Staining with Hoechst (nuclear) and streptavidin-488 (green, anti biotin) and red fluorescence from RFP. Staining as described above. Transfection with MS2-U4 RNA and treatment with ROS-bPNA and biotin-phenol. Co-localization of red and green fluorescence in the nucleus. (Right) Line-trace of fluorescence profile shows high degree of red-green correlation in support of co-localization.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

At various places in the present specification, divalent linking substituents are described. Where the structure clearly requires a linking group, the Markush variables listed for that group are understood to be linking groups.

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. As used herein, the term “substituted” means that a hydrogen atom is removed and replaced by a substituent. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used herein, “Cam alkynyl” refers to an alkyl group having one or more triple carbon-carbon bonds and having n to m carbons. Example alkynyl groups include, but are not limited to, ethynyl, propyn-1-yl, propyn-2-yl, and the like. In some embodiments, the alkynyl moiety contains 2 to 6, 2 to 4, or 2 to 3 carbon atoms.

As used herein, the term “Cₙ₋ₘ alkylene”, employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In some embodiments, the alkylene moiety contains 2 to 6, 2 to 4, 2 to 3, 1 to 6, 1 to 4, or 1 to 2 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include methoxy, ethoxy, propoxy (e.g., n-propoxy and isopropoxy), tert-butoxy, and the like. In some embodiments, the alkyl group has 1 to 6, 1 to 4, or 1 to 3 carbon atoms.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms.

As used herein, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonyl” refers to a group of formula —C(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylcarbonylamino” refers to a group of formula —NHC(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonylamino” refers to a group of formula —NHS(O)₂-alkyl, wherein the alkyl group has n to m carbon atoms. In some embodiments, the alkyl group has 1 to 6, 1 to 4, or 1 to 3 carbon atoms.

As used herein, the term “aminosulfonyl” refers to a group of formula —S(O)₂NH₂.

As used herein, the term “Cₙ₋ₘ alkylaminosulfonyl” refers to a group of formula —S(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonyl” refers to a group of formula —S(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ₋ₘ alkylaminosulfonylamino” refers to a group of formula —NHS(O)₂NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminosulfonylamino” refers to a group of formula —NHS(O)₂N(alkyl)₂, wherein each alkyl group independently has n to m carbon atoms. In some embodiments, each alkyl group has, independently, 1 to 6, 1 to 4, or 1 to 3 carbon atoms.

As used herein, the term “aminocarbonylamino”, employed alone or in combination with other terms, refers to a group of formula —NHC(O)NH₂.

As used herein, the term “Cₙ₋ₘ alkylaminocarbonylamino” refers to a group of formula —NHC(O)NH(alkyl), wherein the alkyl group has n to m carbon atoms. In some embodiments, the alkyl group has 1 to 6, 1 to 4, or 1 to 3 carbon atoms.

As used herein, the term “di(Cₙ₋ₘ alkyl)aminocarbonylamino” refers to a group of formula —NHC(O)N(alkyl)₂, wherein each alkyl group independently has n to m carbon atoms. In some embodiments, each alkyl group has, independently, 1 to 6, 1 to 4, or 1 to 3 carbon atoms.

As used herein, the term “Cₙ₋ₘ alkylcarbamyl” refers to a group of formula —C(O)—NH(alkyl), wherein the alkyl group has n to m carbon atoms. In some embodiments, the alkyl group has 1 to 6, 1 to 4, or 1 to 3 carbon atoms.

As used herein, the term “thio” refers to a group of formula —SH.

As used herein, the term “Cₙ₋ₘ alkylsulfinyl” refers to a group of formula —S(O)-alkyl, wherein the alkyl group has n to m carbon atoms. In some embodiments, the alkyl group has 1 to 6, 1 to 4, or 1 to 3 carbon atoms.

As used herein, the term “Cₙ₋ₘ alkylsulfonyl” refers to a group of formula —S(O)₂-alkyl, wherein the alkyl group has n to m carbon atoms. In some embodiments, the alkyl group has 1 to 6, 1 to 4, or 1 to 3 carbon atoms.

As used herein, the term “amino” refers to a group of formula —NH₂.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to about 20 carbon atoms, from 6 to about 15 carbon atoms, or from 6 to about 10 carbon atoms. In some embodiments, the aryl group is a substituted or unsubstituted phenyl.

As used herein, the term “carbamyl” to a group of formula —C(O) NH₂.

As used herein, the term “carbonyl”, employed alone or in combination with other terms, refers to a —C(═O)— group, which may also be written as C(O).

As used herein, the term “di(Cₙ₋ₘ-alkyl)amino” refers to a group of formula —N(alkyl) 2, wherein the two alkyl groups each has, independently, n to m carbon atoms. In some embodiments, each alkyl group independently has 1 to 6, 1 to 4, or 1 to 3 carbon atoms.

As used herein, the term “di(Cₙ₋ₘ-alkyl) carbamyl” refers to a group of formula —C(O)N(alkyl)₂, wherein the two alkyl groups each has, independently, n to m carbon atoms. In some embodiments, each alkyl group independently has 1 to 6, 1 to 4, or 1 to 3 carbon atoms.

As used herein, the term “halo” refers to F, Cl, Br, or I. In some embodiments, a halo is F, Cl, or Br. In some embodiments, a halo is F or Cl.

As used herein, “Ca-m haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, “cycloalkyl” refers to non-aromatic cyclic hydrocarbons including cyclized alkyl and/or alkenyl groups. Cycloalkyl groups can include mono- or polycyclic (e.g., having 2, 3 or 4 fused rings) groups and spirocycles. Cycloalkyl groups can have 3, 4, 5, 6, 7, 8, 9, or 10 ring-forming carbons (C3.10). Ring-forming carbon atoms of a cycloalkyl group can be optionally substituted by oxo or sulfido (e.g., C(O) or C(S)). Cycloalkyl groups also include cycloalkylidenes. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, and the like. In some embodiments, cycloalkyl is cyclopropyl, cyclobutyl, cyclopentyl, cyclohexyl, cyclopentyl, or adamantyl. In some embodiments, the cycloalkyl has 6-10 ring-forming carbon atoms. In some embodiments, cycloalkyl is adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom including a ring-forming atom of the fused aromatic ring.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl has 5-10 ring atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropurin, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazepine, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl has 4-10, 4-7 or 4-6 ring atoms with 1 or 2 heteroatoms independently selected from nitrogen, oxygen, or sulfur and having one or more oxidized ring members.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas a pyridin-3-yl ring is attached at the 3-position.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

In some embodiments, the compounds described herein can contain one or more asymmetric centers and thus occur as racemates and racemic mixtures, enantiomerically enriched mixtures, single enantiomers, individual diastereomers and diastereomeric mixtures (e.g., including (R)- and(S)-enantiomers, diastereomers, (D)-isomers, (L)-isomers, (+) (dextrorotatory) forms, (−) (levorotatory) forms, the racemic mixtures thereof, and other mixtures thereof). Additional asymmetric carbon atoms can be present in a substituent, such as an alkyl group. All such isomeric forms, as well as mixtures thereof, of these compounds are expressly included in the present description. The compounds described herein can also or further contain linkages wherein bond rotation is restricted about that particular linkage, e.g. restriction resulting from the presence of a ring or double bond (e.g., carbon-carbon bonds, carbon-nitrogen bonds such as amide bonds). Accordingly, all cis/trans and E/Z isomers and rotational isomers are expressly included in the present description. Unless otherwise mentioned or indicated, the chemical designation of a compound encompasses the mixture of all possible stereochemically isomeric forms of that compound.

Optical isomers can be obtained in pure form by standard procedures known to those skilled in the art, and include, but are not limited to, diastereomeric salt formation, kinetic resolution, and asymmetric synthesis. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, IN 1972), each of which is incorporated herein by reference in their entireties. It is also understood that the compounds described herein include all possible regioisomers, and mixtures thereof, which can be obtained in pure form by standard separation procedures known to those skilled in the art, and include, but are not limited to, column chromatography, thin-layer chromatography, and high-performance liquid chromatography.

Unless specifically defined, compounds provided herein can also include all isotopes of atoms occurring in the intermediates or final compounds. Isotopes include those atoms having the same atomic number but different mass numbers. Unless otherwise stated, when an atom is designated as an isotope or radioisotope (e.g., deuterium, [¹¹C], [¹⁸F]), the atom is understood to comprise the isotope or radioisotope in an amount at least greater than the natural abundance of the isotope or radioisotope. For example, when an atom is designated as “D” or “deuterium”, the position is understood to have deuterium at an abundance that is at least 3000 times greater than the natural abundance of deuterium, which is 0.015% (i.e., at least 45% incorporation of deuterium).

All compounds, and pharmaceutically acceptable salts thereof, can be found together with other substances such as water and solvents (e.g. hydrates and solvates) or can be isolated.

In some embodiments, preparation of compounds can involve the addition of acids or bases to affect, for example, catalysis of a desired reaction or formation of salt forms such as acid addition salts.

Example acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Example bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

### Bifacial Peptide Nucleic Acid Probes

Described herein are bifacial peptide nucleic acid probes. These probes can include a triplex hybrid forming moiety (e.g., a plurality of binding motifs disposed along a peptidyl backbone) conjugated to a prosthetic group. The triplex hybrid forming moiety can bind to a non-canonical base pairing site in a target nucleic acid via triplex hybridization. In this way, a variety of prosthetic groups (e.g., dyes, labels, reactive groups, etc.) can be selectively delivered to a target nucleic acid (e.g., in vivo, in vitro, or ex vivo).

In some embodiments, the bifacial peptide nucleic acid probe can be defined by Formula I below

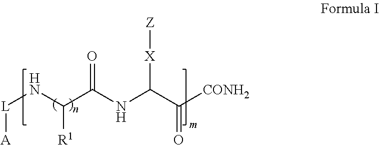

wherein
A represents a prosthetic group;
X is absent or represents a first bivalent linking group;
L is absent or represents a second bivalent linking group;
n is, individually for each occurrence, an integer selected from 1 and 2;
m is an integer selected from 2, 3, and 4;
Z represents, individually for each occurrence, a binding motif selected from one of the following

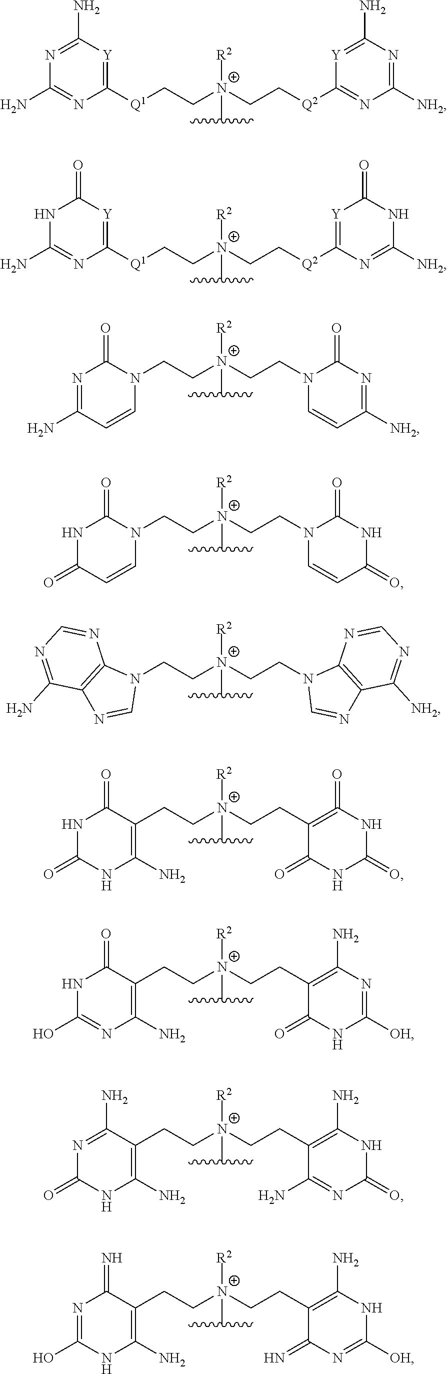

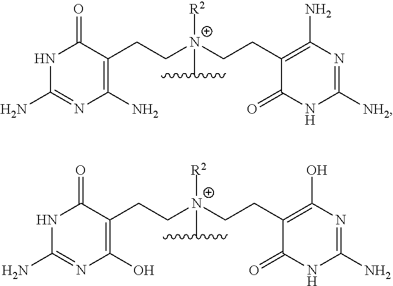

Q¹ and Q² individually represent —O— or —NR^{A}—;
Y is N, —CR^{B}—,
R¹ is, individually for each occurrence, selected from one of the following

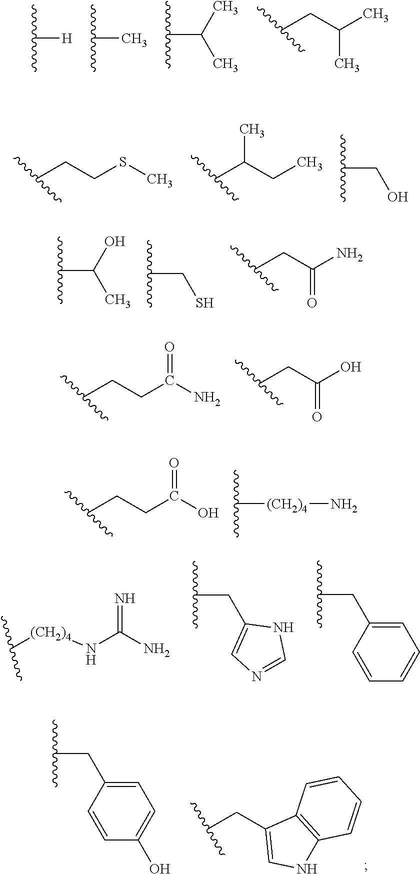

R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In some embodiments, R^{A} represents H for each occurrence (e.g., Q¹ and Q² individually represent —O— or —NH—). In some embodiments, Q¹ and Q² represent —O— for each occurrence. In other embodiments, Q¹ and Q² represent —NH— for each occurrence.

In some embodiments, R² represents H for each occurrence.

In some embodiments, R¹ is, individually for each occurrence, selected from one of the following

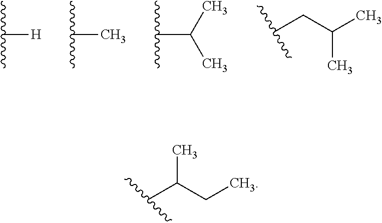

In certain embodiments, R¹ is, individually for each occurrence, —H or —CH₃.

In some embodiments, m is 2. In other embodiments, m is 3.

In some embodiments, n is 1 in all occurrences. In other embodiments, m is 3 and n is 1 in two occurrences and n is 2 in one occurrence.

In some embodiments, X represents a first bivalent linking group. In some embodiments, the first bivalent linking group comprises from 3 to 20 atoms, such as from 3 to 16 atoms or from 3 to 12 atoms. In certain embodiments, the first bivalent linking group comprises an alkylene linker or a heteroalkylene linker.

In some embodiments, L represents a second bivalent linking group. In some embodiments, the second linking group comprises from 3 to 36 atoms, such as from 3 to 24 atoms or from 3 to 16 atoms. In certain embodiments, the second bivalent linking group comprises an alkylene linker or a heteroalkylene linker.

In some embodiments, in at least one occurrence, Z represents the binding motif shown below

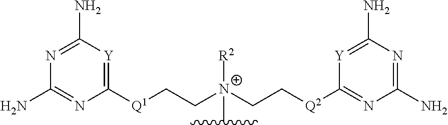

wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In certain embodiments, m is 2 and Z represents the binding motif shown below in two occurrences

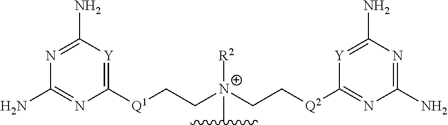

wherein
Q¹ and Q² individually represent —O— or —NR^{A}:
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In certain embodiments, m is 3 and Z represents the binding motif shown below in two occurrences

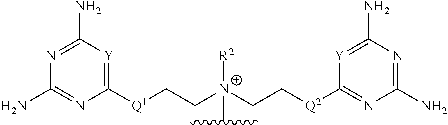

wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

In certain examples, the compound is defined by Formula IA below

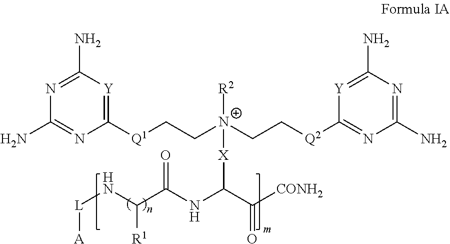

wherein A, L, Y, Q¹, Q², X, n, and m are as defined above

In certain examples, the compound is defined by Formula IB

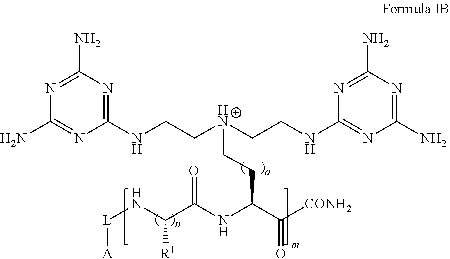

wherein
A, L, R¹, m, and n are as defined above; and
a is, individually for each occurrence, an integer selected from 3, 4, 5, and 6.

### Linking Groups

In the compounds above, the linking groups (e.g., the first linking group and/or the second linking group), when present, can be any suitable group or moiety which can function as a bivalent linker connecting the prosthetic moiety to the peptidyl backbone. The linking group can be composed of any assembly of atoms, including oligomeric and polymeric chains. In some cases, the total number of atoms in the linking group can be from 3 to 200 atoms (e.g., from 3 to 150 atoms, from 3 to 100 atoms, from 3 and 50 atoms, from 3 to 25 atoms, from 3 to 15 atoms, or from 3 to 10 atoms).

In some embodiments, the linking group can be, for example, an alkyl, alkoxy, alkylaryl, alkylheteroaryl, alkylcycloalkyl, alkylheterocycloalkyl, alkylthio, alkylsulfinyl, alkylsulfonyl, alkylamino, dialkylamino, alkylcarbonyl, alkoxycarbonyl, alkylaminocarbonyl, dialkylaminocarbonyl, or polyamino group. In some embodiments, the linking group can comprise one of the groups above joined to one or both of the moieties to which it is attached by a functional group. Examples of suitable functional groups include, for example, secondary amides (—CONH—), tertiary amides (—CONR—), secondary carbamates (—OCONH—; —NHCOO—), tertiary carbamates (—OCONR—; —NRCOO—), ureas (—NHCONH—; —NRCONH—; —NHCONR—, or —NRCONR—), carbinols (—CHOH—, —CROH—), ethers (—O—), and esters (—COO—, —CH₂O₂C—, CHRO₂C—), wherein R is an alkyl group, an aryl group, or a heterocyclic group. For example, in some embodiments, the linking group can comprise an alkyl group (e.g., a C₁-C₁₂ alkyl group, a C₁-C₈ alkyl group, or a C₁-C₆ alkyl group) bound to one or both of the moieties to which it is attached via an ester (—COO—, —CH₂O₂C—, CHRO₂C—), a secondary amide (—CONH—), or a tertiary amide (—CONR—), wherein R is an alkyl group, an aryl group, or a heterocyclic group. In certain embodiments, the linking group can be chosen from one of the following:

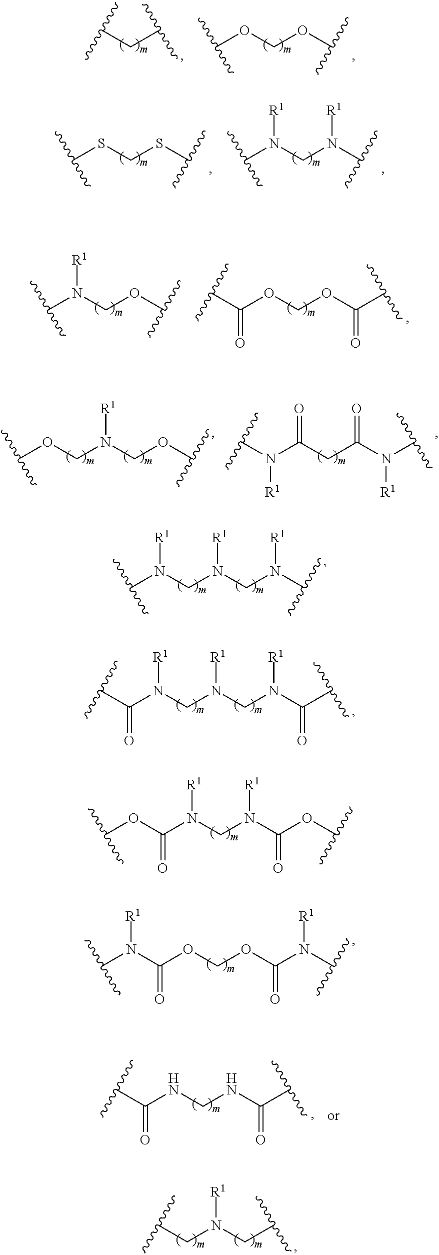

where m is an integer from 1 to 12 and R¹ is, independently for each occurrence, hydrogen, an alkyl group, an aryl group, or a heterocyclic group.

In some embodiments, the linking group can be

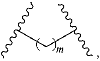

where m is an integer from 1 to 12 (e.g., an integer from 1 to 6, or an integer from 1 to 3). In certain embodiments, the linking group can be

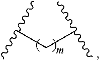

where m is 1.

If desired, the linker can serve to modify the solubility of the compounds described herein. In some embodiments, the linker is hydrophilic. In some embodiments, the linker can be an alkyl group, an alkylaryl group, an oligo- or polyalkylene oxide chain (e.g., an oligo- or polyethylene glycol chain), or an oligo- or poly(amino acid) chain.

### Prosthetic Groups

In the compounds above, the prosthetic group can comprise any functional moiety which can be delivered as cargo using the bifacial peptide nucleic acid probes described herein.

For example, the prosthetic group can comprise a fluorogenic dye (e.g., a thiazole derived dye such as thiazole orange, dimethylindole red or a derivative thereof, a symmetric cyanine dye, an asymmetric cyanine dye, a fluorescein, a rhodamine, a fluorogenic variant of an asymmetric cyanine dye or fluorescein such as JF635 and JF646, an arsenate dye such as FIASH and ReASH, malachite green or a derivative thereof, a coumarin dye, or a hydroxybenzylidene dye), a protein ligand (e.g., a ligand for a protein which facilitates/drives association of a protein of interest with a target nucleic acid bound to the bifacial peptide nucleic acid probe and/or other proteins bound to the target nucleic acid, such as a ubiquitin ligase ligand (e.g., a ligand that binds to an E3 ligase such as XIAP, VHL, cereblon, and MDM2), a ligand for a translational activator, a ligand for a translational inhibitor, a ligand for a transcription activator, a ligand for a transcription inhibitor, a ligand for a nuclease, or a ligand for a cell surface protein), a redox-active center, an ROS-generating center (e.g., Cu-Phen or Fe-EDTA), a photoreactive center (e.g., a photoreactive organic moiety (e.g., benzophenone) that generate radicals upon activation with actinic radiation, an ethylenically unsaturated moiety that induces crosslinking), a spin label (e.g., a paramagnetic metal center or stable radical that assists in spectroscopic analysis of a target nucleic acid bound to the bifacial peptide nucleic acid probe and/or other proteins bound to the target nucleic acid), a group transfer agent (e.g., an active ester that transfers to a protein bound to the target nucleic acid to which the bifacial peptide nucleic acid probe is bound, thereby deactivating the protein), a therapeutic agent (e.g., a therapeutic, diagnostic, or prophylactic agent), a catalytic center, a click motif (e.g., a functional group that can participate in a click chemistry to allow for subsequent conjugation of another moiety bearing a complementary click motif to the bifacial peptide nucleic acid probe), or an NMR active label.

In some embodiments, the prosthetic group is not cyanine 5 (Cy5), cyanine 3 (Cy3), or carboxyfluorescein (Cbf).

### Methods of Use

The bifacial peptide nucleic acid probes described herein can be used to localize any suitable prosthetic group with a target nucleic acid of interest (which itself can be associated with other biomolecules in vivo, such as proteins). Depending on the nucleic acid and the nature of the prosthetic group, the prosthetic group can then be utilized for some functionality (e.g., for labeling the target nucleic acid, for reacting with the target nucleic acid, for labeling a protein associated with the target nucleic acid, and/or for reacting with a protein associated with the target nucleic acid.

For example, provided herein are methods of associating a prosthetic group with a target nucleic acid. These methods can comprise contacting the nucleic acid with a bifacial peptide nucleic acid probe comprising a triplex hybrid forming moiety conjugated to a prosthetic group. The bifacial peptide nucleic acid probe can bind to a non-canonical base pairing site in the target nucleic acid via triplex hybridization, thereby associating the prosthetic group with the target nucleic acid.

Also provided herein are methods of associating a prosthetic group with a target protein. These methods can comprise contacting a nucleic acid with a bifacial peptide nucleic acid probe comprising a triplex hybrid forming moiety conjugated to a prosthetic group. The bifacial peptide nucleic acid probe can bind to a non-canonical base pairing site in the target nucleic acid via triplex hybridization. The nucleic acid can form a ribonucleoprotein complex with the target protein, thereby associating the prosthetic group with the target protein.

Also provided are method of detecting a target nucleic acid. These methods can comprise contacting the target nucleic acid with a bifacial peptide nucleic acid probe comprising a triplex hybrid forming moiety conjugated to a fluorogenic dye, a spin label, a therapeutic agent (e.g., a diagnostic agent), or an NMR active label. The bifacial peptide nucleic acid probe can bind to a non-canonical base pairing site in the target nucleic acid via triplex hybridization, thereby associating the fluorogenic dye, the spin label, the therapeutic agent (e.g., the diagnostic agent), or the NMR active label with the target nucleic acid. The fluorogenic dye, the spin label, the therapeutic agent (e.g., the diagnostic agent), or the NMR active label can then be interrogated (e.g., visually or spectroscopically) to detect the target nucleic acid.

Also provided are methods of selectively degrading a target protein in vivo. These methods can comprise contacting a nucleic acid with a bifacial peptide nucleic acid probe comprising a triplex hybrid forming moiety conjugated to a ubiquitin ligase ligand. The bifacial peptide nucleic acid probe can bind to a non-canonical base pairing site in the target nucleic acid via triplex hybridization. The nucleic acid can form a ribonucleoprotein complex with the target protein, thereby associating the ubiquitin ligase ligand with the target protein. The ubiquitin ligase ligand of the bifacial peptide nucleic acid probe bound to the nucleic acid can then direct ubiquitination of the target protein, thereby directing proteolysis of the target protein.

Also provided are methods of selectively labeling a target protein in vivo. These methods can comprise contacting a nucleic acid with a bifacial peptide nucleic acid probe comprising a triplex hybrid forming moiety conjugated to a redox-active center, an ROS-generating center, a photoreactive center, or a catalytic center. The bifacial peptide nucleic acid probe can bind to a non-canonical base pairing site in the target nucleic acid via triplex hybridization. The nucleic acid can form a ribonucleoprotein complex with the target protein. The redox-active center, the ROS-generating center, the photoreactive center, or the catalytic center of the bifacial peptide nucleic acid probe bound to the nucleic acid can then participate in a chemical reaction that results in covalent modification of the target protein, thereby labeling the target protein.

By way of example, in some embodiments, the bifacial peptide nucleic acid probe comprises a triplex hybrid forming moiety conjugated to an ROS-generating center. The nucleic acid can form a ribonucleoprotein complex with the target protein, and the ROS-generating center can generate radical species that react with biotin-phenol to generate a phenoxy radical which reacts with the target protein, thereby labeling the target protein with biotin.

In the methods above, the bifacial peptide nucleic acid probe can bind to a non-canonical base pairing site in a nucleic acid via triplex hybridization. In certain embodiments, the nucleic acid comprises RNA. In certain embodiments, the non-canonical base pairing site comprises a U-rich internal loop (URIL). The URIL can be a loop that includes at least four non-canonical base pairs, wherein at least 50% of the non-canonical base pairs comprise U—U pairs. In some examples, the URIL comprises from 4-8 non-canonical base pairs.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Intracellular RNA and DNA Tracking by Uridine Rich Internal Loop Tagging with Fluorogenic bPNA

The most widely used method for intracellular RNA fluorescence labeling is MS2 labeling, which generally relies on the use of multiple protein-labels targeted to multiple RNA (MS2) hairpin structures installed on the RNA of interest (ROI). While effective and conveniently applied in cell biology labs, the protein labels add significant mass to the bound RNA, which potentially impacts steric accessibility and native RNA biology. We have previously demonstrated that internal, genetically encoded, uridine-rich internal loops (URILs) comprised of 4 contiguous UU pairs (8 nt) in RNA may be targeted with minimal structural perturbation by triplex hybridization with 1 kD bifacial Peptide Nucleic Acids (bPNAs). A URIL-targeting strategy for RNA and DNA tracking would avoid the use of cumbersome protein-fusion labels and minimize structural alterations to the RNA of interest. Here we show that URIL-targeting fluorogenic bPNA probes in cell media can penetrate cell membranes and effectively label RNAs and RNPs in fixed and live cells. This method, which we call Fluorogenic U-rich Internal Loop (FLURIL) tagging, was internally validated through the use of RNAs bearing both URIL and MS2 labeling sites. Notably, direct comparison of CRISPR-dCas labeled genomic loci in live U2OS cells revealed that FLURIL tagged gRNA yielded loci up to 7× brighter than loci targeted by guide RNA modified with an array of 8 MS2 hairpins. Together, these data show that FLURIL tagging provides a versatile scope of intracellular RNA and DNA tracking in a light molecular footprint, while maintaining compatibility with existing methods

### INTRODUCTION

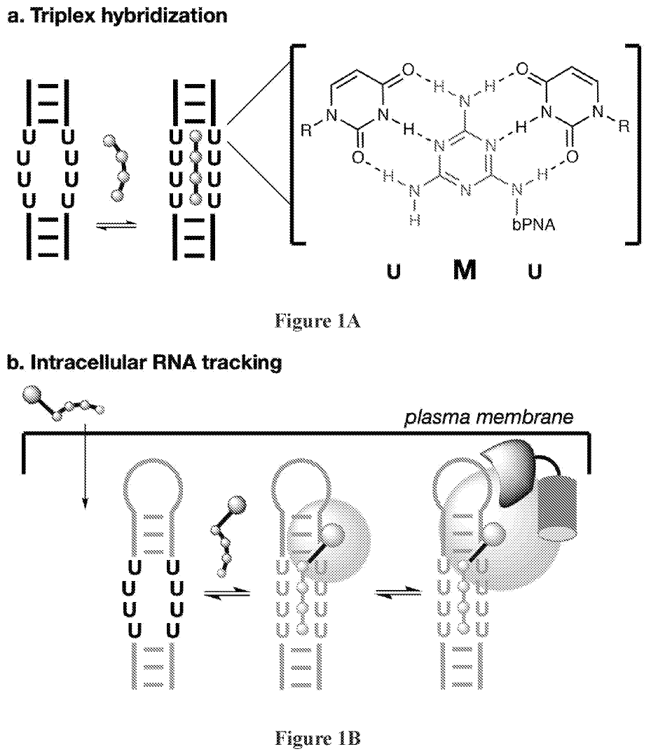

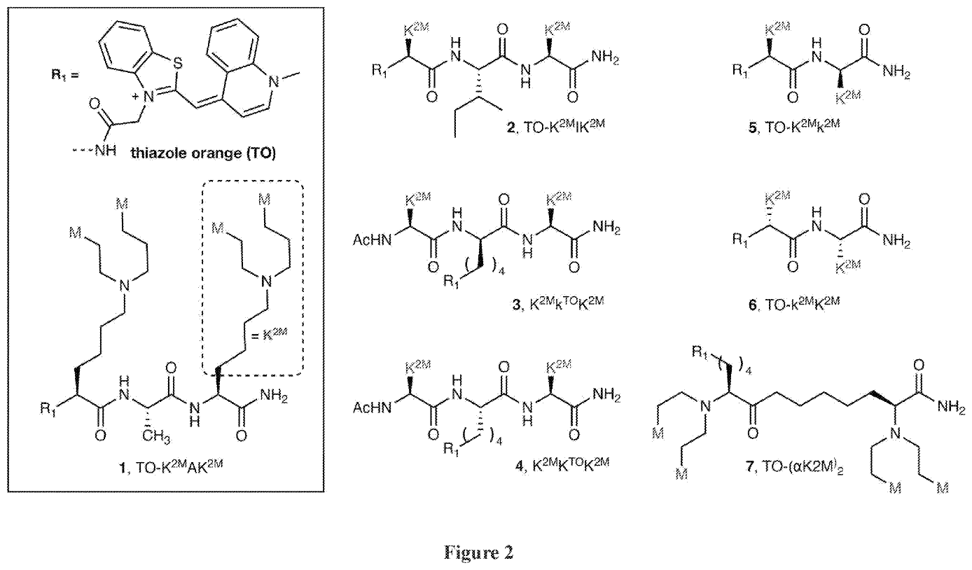

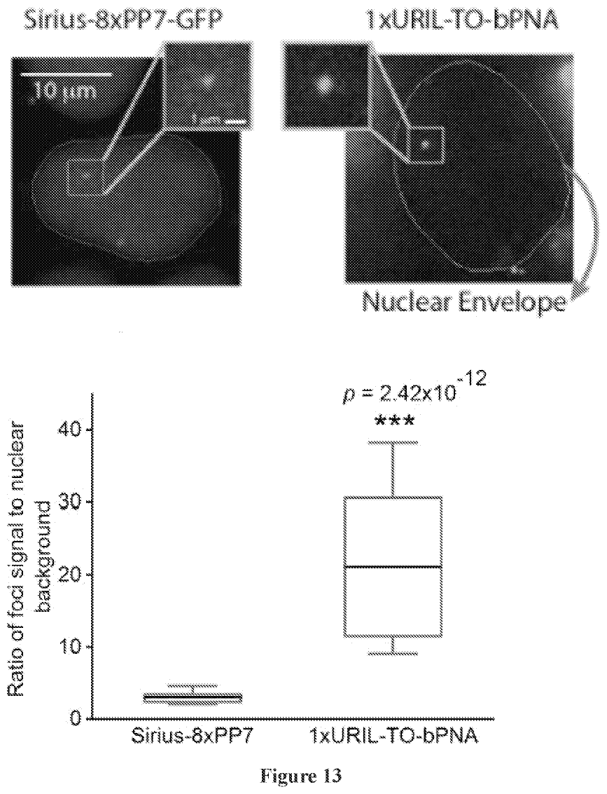

In this example, we show that intracellular RNAs bearing an 8-nt (U₄ₓU₄) U-rich internal loop (URIL) can be selectively labeled with a fluorogenic bPNA probe via triplex hybridization, in a method we call Fluorogenic U-rich internal loop (FLURIL) tagging. The FLURIL system minimizes the introduction of new RNA secondary structures by duplex-for-triplex stem replacement and is considerably smaller than a typical aptamer or protein-labeling systems for RNA, yet has improved brightness and stability (FIG. 13). We have developed a bifacial peptide nucleic acid (bPNA), which presents the synthetic melamine base on an α-peptide backbone (FIG. 1). The bPNA family of compounds selectively hybridize with U_{nX}Uₙ internal bulges via formation of uracil-melamine-uracil (UMU) base triples, forming in triplex stems that can functionally replace native RNA stems, tertiary contacts, block protein readthrough, direct chemistry, and modulate lncRNA lifetime. Optimized cationic 4 M bPNAs (with 4 melamine bases, FIG. 2) are cell-permeable and can bind to structured U₄ₓU₄ bulges while retaining nanomolar affinity, thus enabling specific intracellular targeting of ROIs that have been engineered to contain a U₄ₓU₄ URIL in the RNA scaffold. This URIL site forms a triplex stem upon hybridization with 4 M bPNA that is structurally similar to a 4 bp native duplex RNA stem. Thus, URIL-tagging with bPNA can drive placement of any sterically-acceptable prosthetic group into an internal site within the RNA fold without the need for aptamer selection; this example describes the targeting of fluorogen-modified bPNA to the URIL site, which we call FLURIL-tagging. We demonstrate FLURIL-tagging of intracellular RNAs in both fixed and live cell contexts.

### Results

Design and synthesis of fluorogenic bPNA probes for URIL RNAs. Cyanine dye (Cy3, Cy5) modified bPNAs exhibit fluorescence enhancement upon triplex hybridization to URIL RNA and subsequent hybrid RNA-protein binding, as presaged by observations with DNA binding. Thiazole orange has been more widely used to signal intercalative binding: sequence-selective “forced intercalation” (FIT) emission with PNA-thiazole orange (TO) conjugates have been demonstrated that place TO at the hybridization interface. Similarly, TO can be attached to pyrrole-imidazole polyamide (Py-Im) minor groove intercalators that fluoresce upon sequence recognition. While these probes have utility, they are limited with regard to transport and RNA binding.

We have synthesized bPNAs N-terminated with Cy5, difluorohydroxybenzylidene (DFHBI) and TO derivatives (FIG. 2). Compact dipeptide or tripeptide bPNA scaffolds containing just 4 melamine base-tripling units (4 M bPNAs) can exhibit high affinity binding to a structured U₄ₓU₄ internal bulge. Structure-function studies found highly effective nucleic acid binding with the use of a lysine derivative (K^{2M}) that presents two melamine bases on the ε-amine; the resulting tertiary amino sidechain becomes cationic upon protonation, obviating the need for additional solubilizing groups and providing electrostatic stabilization of nucleic acid binding. Even following N-terminal modification with fluorogenic modules, these efficient bPNA probes are in a low molecular weight regime (~1 kD). Moreover, Fmoc-K^{2M} can be obtained on multigram scale by double reductive alkylation of Fmoc-lysine with melamine acetaldehyde without column purification; when applied in a di or tripeptide backbone, these bPNA probes are highly accessible and synthetically scalable.

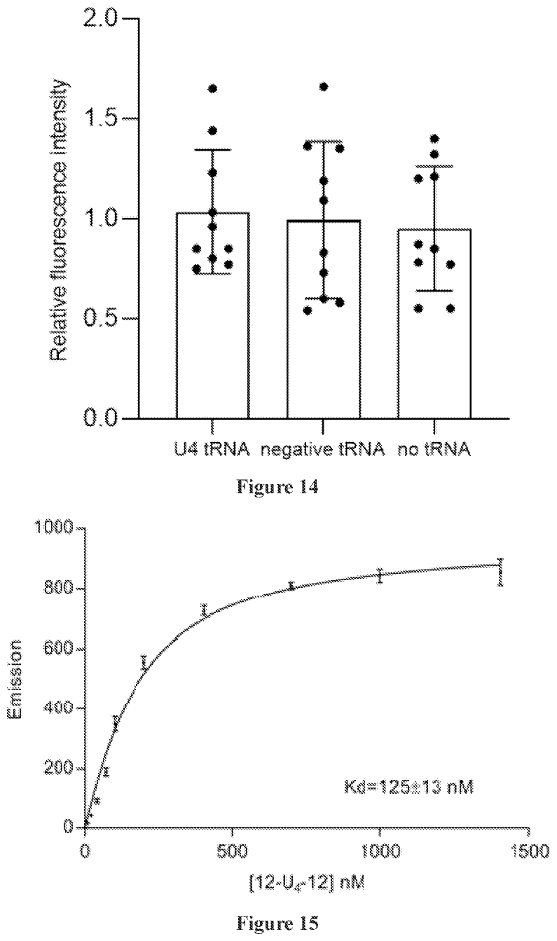

We prepared a small family of 4 M bPNAs (FIG. 2) based on prior structure-function studies, focusing primarily on the tripeptide (K^{2M}X-K^{2M}) scaffold where X is an α-amino acid, and dipeptide isomers. Within the tripeptide K^{2M}Ala-K^{2M} we tested Cy3, Cy5, DFHBI and thiazole orange (TO) dyes at the N-terminus. Of these, the turn-on upon RNA complexation was modest for the cyanine and DFHBI dyes (FIGS. 14 and 15); we thus focused on TO derivatives. In K^{2M}X-K^{2M}, thiazole orange carboxylic acid was coupled via a β-alanine linker to the N-terminus of (X=Ala, Ile) or directly to the α-amine of lysine (X=K^{TO}). Dipeptide and tripeptide bPNAs of alternating amino acid configuration show an advantage in triplex hybridization over the homochiral dipeptides due to syndiotactic base presentation and we set out to test the impact of stereochemistry on fluorogenic binding when modified with TO on the N-terminus (TO-K^{2M}K^{2M}, TO-K^{2M}K^{2M}) and ε-amine of the central amino acid (K^{2M}k^{TO}-K^{2M}). Similarly, we synthesized the isopeptide (TO-(αk^{2M})₂) that features a sidechain linkage, base presentation on the α-nitrogens and direct TO modification on the ε-amine.

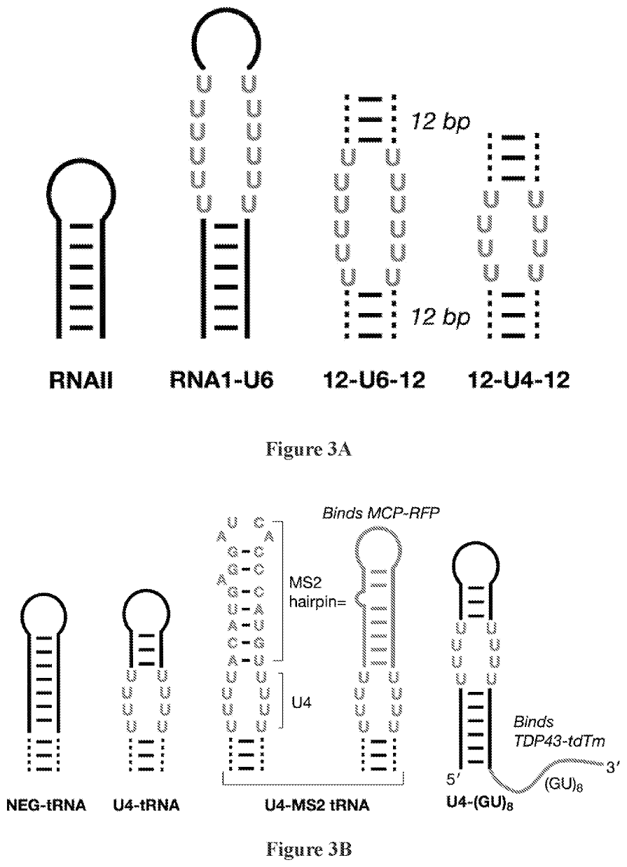

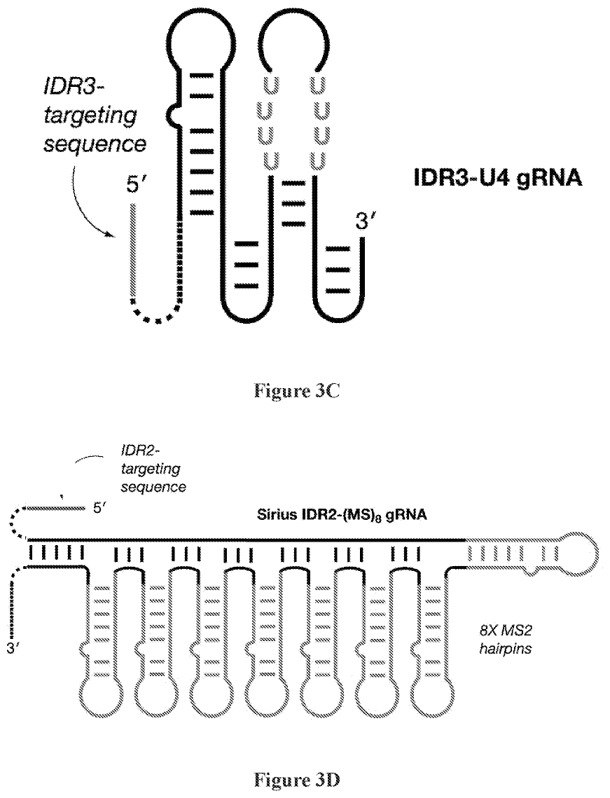

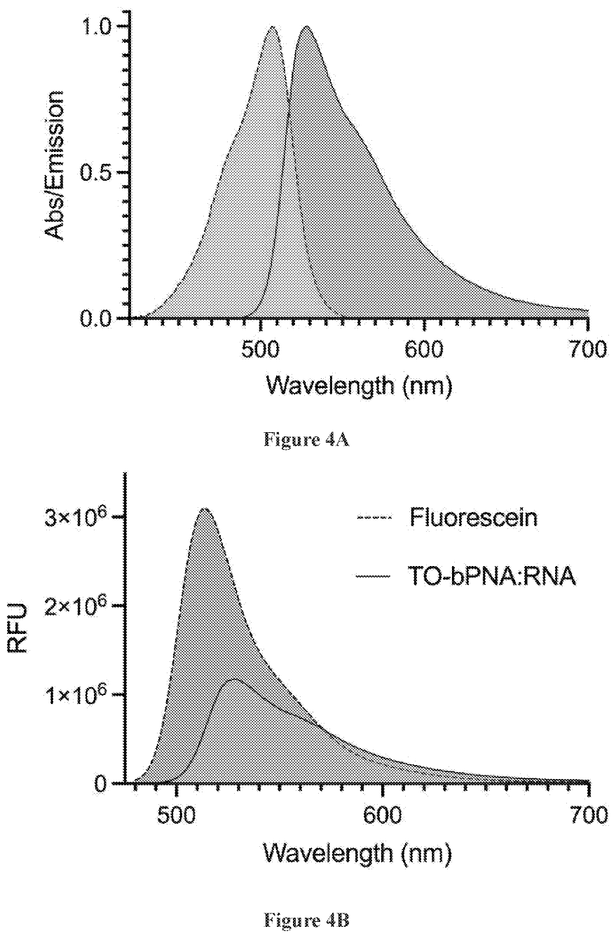

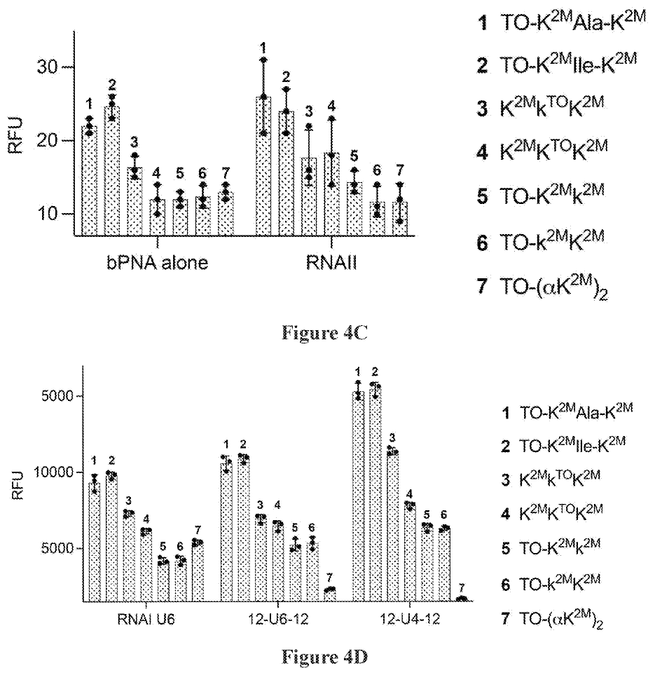

In vitro evaluation of fluorogenic URIL-RNA binding by bPNA variants. With these URIL-RNAs and bPNAs in hand, we tested fluorogenic binding in vitro to hairpin and duplex RNAs that were designed to present U-rich internal loops (URILs) in varying contexts (FIG. 3A). The absorbance and emission properties of the free TO-bPNAs were similar to the parent thiazole orange dye, which exhibits weak emission in solution (quantum yield ~10⁻⁴). However, when TO-K^{2M}Ala-K^{2M} binds to URIL RNAs (FIG. 2A) emission increases by up to 600 fold (FIG. 4). Indeed, the TO-bPNA:RNA hybrid exhibited a robust relative quantum yield of 43% (FIG. 3B). This TO-bPNA:URIL hybrid quantum yield exceeds that observed with the RNA aptamer Mango bound to TO-biotin (quantum yield=14%). An RNA hairpin construct with a fully base-paired duplex stem (RNAII) does not improve the brightness of the TO-bPNA derivatives (FIG. 4C). While thiazole orange fluorescence turn-on upon RNA binding is expected for non-specific intercalation, this only occurs at higher concentrations; the null response with non-URIL RNA (RNAII) indicates the critical role of bPNA triplex hybridization in fluorogenic binding. All bPNAs gave a strong fluorogenic response to URIL RNAs, but there appeared to be significant differences among bPNA variants. Further, despite the biophysical advantage previously observed for triplex hybridization with L,D and D,L dipeptide bPNAs, these fluorogenic variants were not as bright as the tripeptides (K^{2M}AlaK^{2M}, K^{2M}Ilek^{2M}). Based on these results, we chose the TO-K^{2M}AlaK^{2M} probe (1) for subsequent intracellular studies.

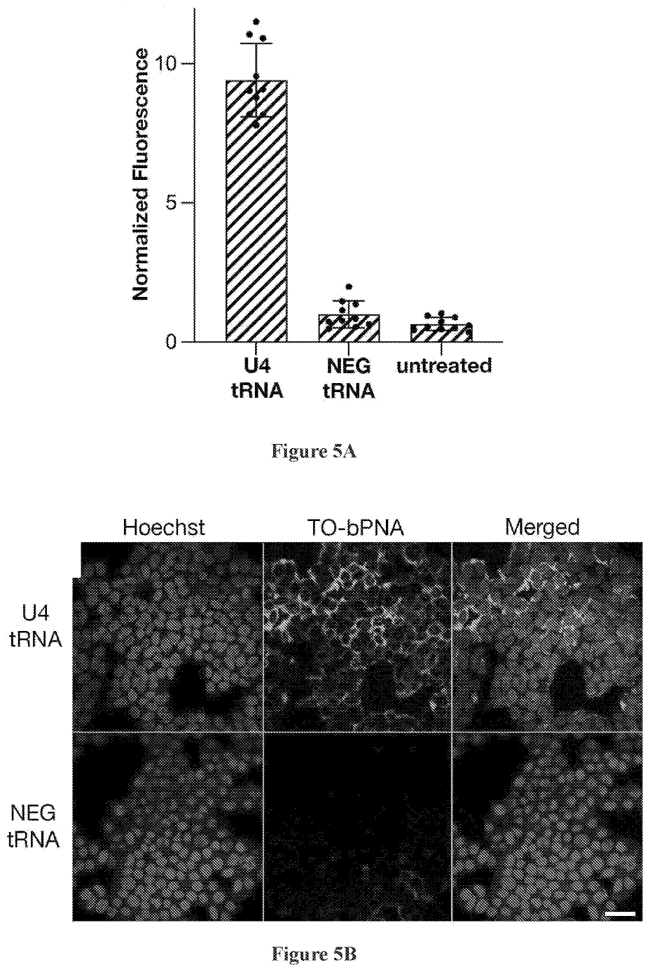

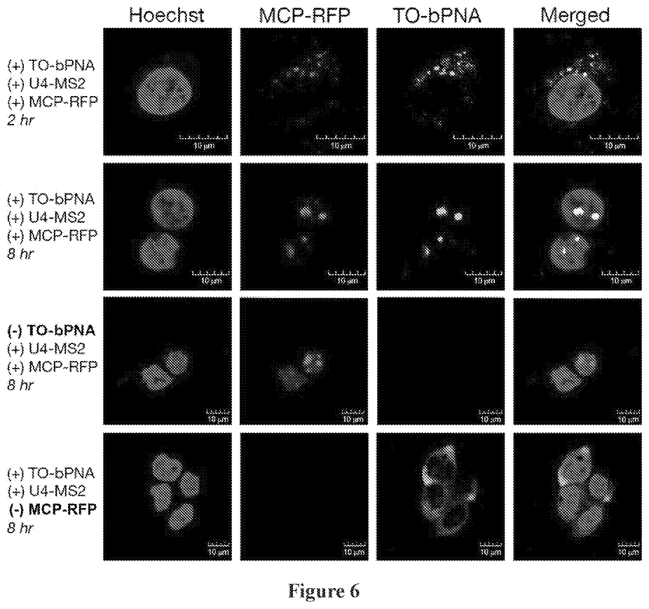

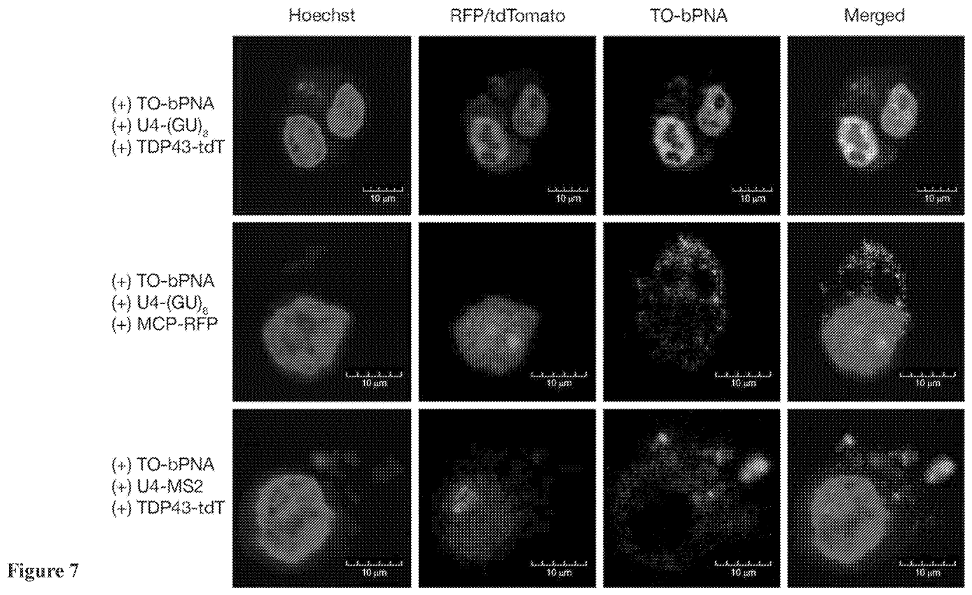

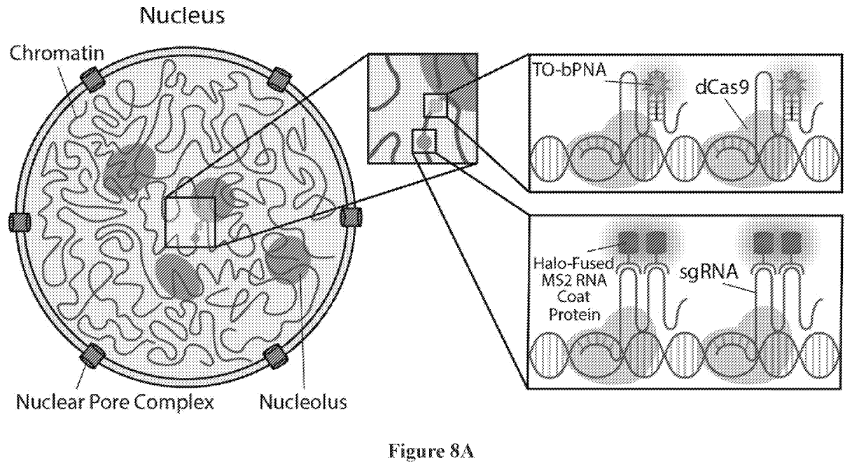

Intracellular labeling of URIL RNAs and bacteriophage RNPs with fluorogenic bPNA. Using similar design principles as the in vitro studies, URIL RNA hairpins (FIG. 2B) were engineered to replace the anticodon loop of a tRNA^{Lys} platform that affords stable intracellular RNA expression upon plasmid transfection into HEK-293T cells. In analogy to the RNAII in vitro control, a fully base-paired anticodon stem (NEG tRNA) was designed to serve as an intracellular negative control that lacks bPNA binding. A bPNA-binding target was provided by the U4-tRNA construct, which was used to determine intracellular fluorogenic triplex hybridization with TO-bPNA. Indeed, treatment of HEK-293T cells with probe 1 in cell culture media following transfection with either U4 or NEG tRNA resulted in a 5-10 fold brighter fluorescence intensity in the U4-tRNA transfected cells (FIG. 5). These data were supportive of intracellular targeting of the engineered URIL RNA, albeit with diminished enhancement relative to in vitro conditions.

To benchmark bPNA labeling of RNA against known RNA tracking strategies, we juxtaposed the U4 URIL with the MS2 hairpin sequence in the tRNA^{Lys} anticodon loop to yield U4-MS2 tRNA (FIG. 2B). The U4-MS2 tRNA was co-transfected into HEK-293T cells with a plasmid encoding an MCP-RFP fusion, which also bears a nuclear localization signal (NLS). Cells imaged two hours after transfection and treatment in media with 1 revealed clear co-localization of red and green fluorescence, supportive of labeling of the U4-MS2 tRNA with TO-bPNA and subsequent binding of the MCP-RFP protein fusion to the triplex hybrid in the cytoplasm (FIG. 6). This red-green co-localization was accompanied by a condensation of signal intensity, possibly due to RFP aggregation. Notably, imaging 8 hours after transfection indicated that both green and red fluorescence had co-localized to the nucleus, consistent with time-dependent transport of both MCP-RFP (which bears the NLS) along with the TO-bPNA hybrid with U4-MS2 tRNA. Thus, treatment of cell culture with TO-bPNA in media effectively labels intracellular URIL RNAs, associated complexes with RNA binding proteins and further, can track intracellular transport of these complexes. Importantly, in the absence of MCP-RFP, green fluorescence from TO-bPNA remains cytoplasmic; in the absence of TO-bPNA, MCP-RFP remains localized to the nucleus as expected.

Intracellular labeling of mammalian RNPs with fluorogenic bPNA. To complement the bacteriophage RBP system, we conducted the identical experiment with a native mammalian RNA and protein pair to verify intracellular targeting by TO-bPNA. There have been extensive studies on TAR DNA/RNA binding protein (TDP-43) due to its central role in the progression of neurodegenerative diseases such as ALS, where it is mislocalized from the nucleus to the cytoplasm. As repeat sequences of UG/TG are established as native RNA partners for TDP-43 repeat sequence, we co-transfected HEK-293T cells with plasmids encoding a URIL hairpin pendant to a UG repeat (U4-(GU)₈, FIG. 3B) and TDP-43-tdTomato. Upon incubation with TO-bPNA in cell media as before, this protocol resulted in the co-localization of red (tdTomato) and green fluorescence in the nucleus (FIG. 7). This is again supportive of TO-bPNA reporting on the location of a mammalian RNA-protein complex in the correct subcellular compartment. With the MS2/MCP and (GU)₈/TDP-43 systems verified, we transfected with swapped RNA-protein pairings that should not result in complex formation: U4-MS2 was co-transfected with TDP-43-tdTomato, and U4-(GU)₈ with MCP-RFP (FIG. 7). In these experiments, both red fluorescent proteins were retained in the nucleus, but the TO-bPNA tagged RNAs remained cytoplasmic, indicating that RNA-protein binding is driving co-localization of red and green fluorescence signals.

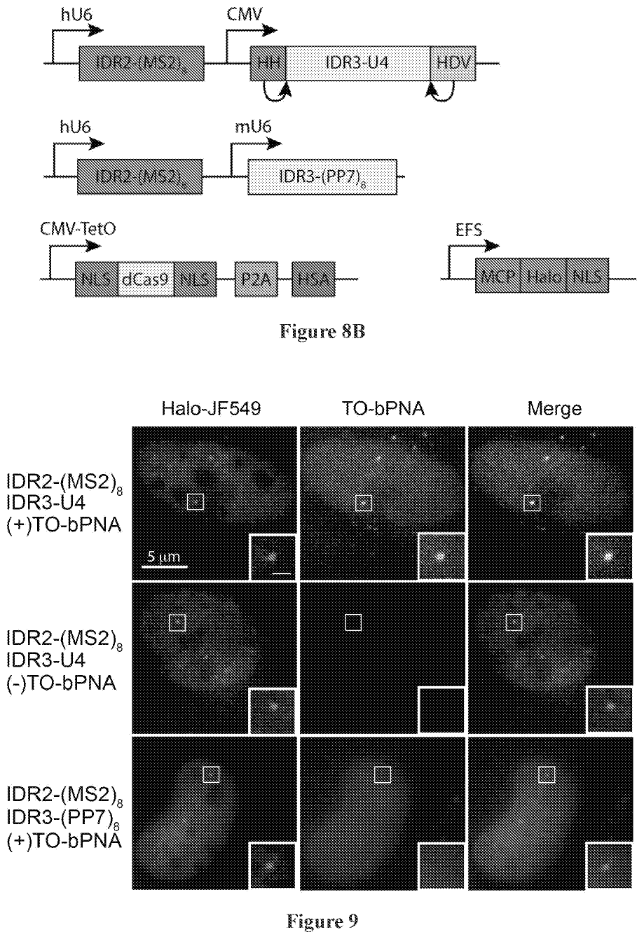

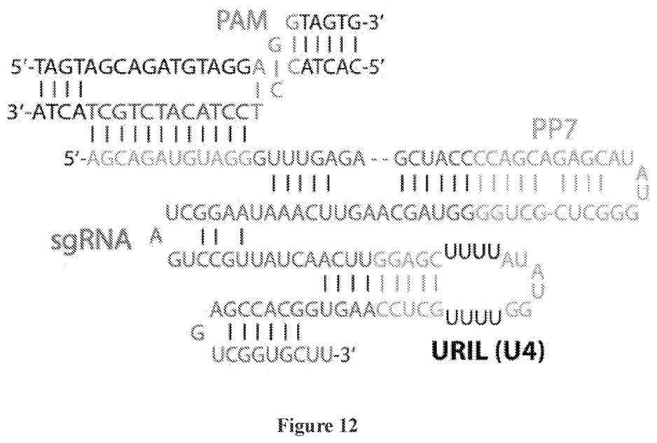

Live cell tracking of genomic loci with bPNA-labeled CRISPR-dCas/gRNA complexes. To test the scope of bPNA cell imaging, we incorporated TO-bPNA reporting into an established approach for live cell genomic loci tracking. CRISPRainbow is a multiplexed, live cell imaging strategy that uses MS2 or PP7 modified guide RNA (gRNA) complexed to deactivated Cas9 (dCas9) to precisely track endogenous DNA repeats marking specific genomic loci. Incorporation of MS2/PP7 bacteriophage RNA hairpin sequences into the gRNA enables the dCas-gRNA complexes to be tracked in live cells by fluorescence imaging upon co-expression of phage coat protein (MCP/PCP) fusions with a “rainbow” of fluorescent proteins; display of multiple MS2/PP7 hairpins in a sequence-optimized array yielded more stable and brighter imaging systems known as CRISPR-Sirius gRNAs. We thus selected two CRISPRainbow/Sirius gRNAs that target proximal intergenic DNA regions IDR2 and IDR3 and modified one to utilize FLURIL-tagging only, with the other serving as an internal benchmark of MS2 labeling. On the gRNA targeting IDR3, one bacteriophage hairpin domain was deleted and replaced with a single U₄XU₄ bulge (URIL) in a stabilized hairpin (FIG. 3C, FIG. 12), rendering the construct (IDR3-U4) trackable by FLURIL-tagging. The IDR3-U4 gRNA carries a PP7 hairpin that serves as an internal control for labeling experiments that lack bPNA probe, and was indeed found to be unreactive to labeling in the absence of TO-bPNA. To target IDR2, a CRISPR-Sirius gRNA called Sirius-IDR2-(MS2)₈ was used (FIG. 3D), which features an array of 8 MS2 hairpins optimized for stability. A plasmid was constructed encoding both IDR3-U4 and Sirius-IDR2-(MS2); and this vector was transfected into U2OS cells stably expressing dCas9 and MCP-HaloTag. Staining of live cells in culture with TO-bPNA and HaloTag-JF549 dye thus enabled simultaneous and orthogonal imaging of IDR3 and IDR2, respectively (FIG. 8A).

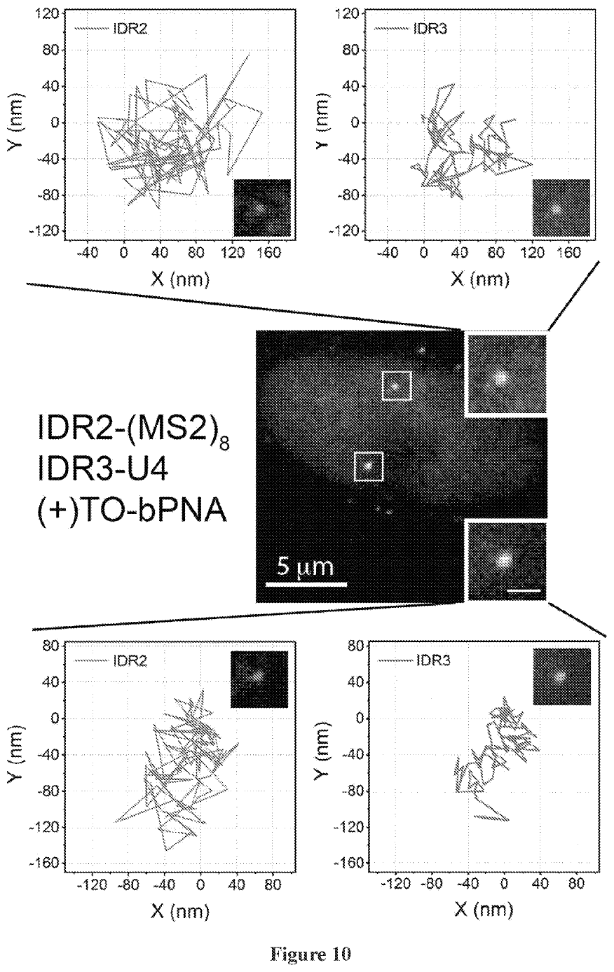

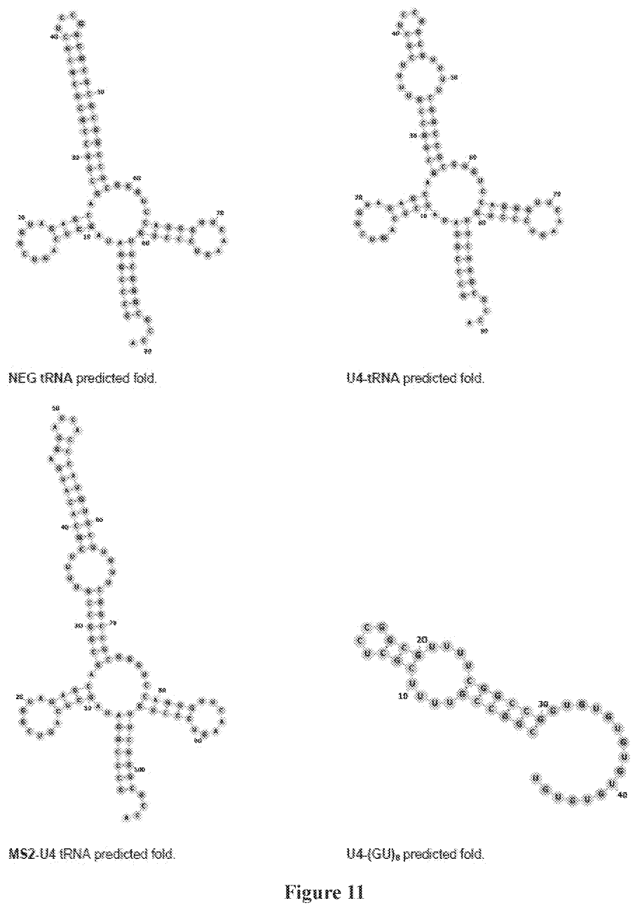

Final vector design was driven by the low efficiency of initial efforts in expression of the modified gRNAs. We speculated that the oligo-U domains triggered early termination by RNA Pol III, which is known to significantly reduce the gRNA targeting efficiency in the CRISPR system. To bypass this issue, we expressed gRNA under a CMV Pol II promoter and ensured precise transcript generation by flanking the gRNA sequence with Hammerhead (HH) and Hepatitis Delta Virus (HDV) self-cleaving ribozymes. We thus modified the gRNA plasmid to contain the Hammerhead (HH) and Hepatitis delta virus (HDV) ribozymes on either side of the gRNA under a CMV promoter, and inserted it into a dual gRNA plasmid expressing CRISPR-Sirius gRNA (Sirius-IDR2-(MS) s) for dual-color genomic loci targeting (FIG. 8). Indeed, the resulting dual color genomic loci labeling system for IDR2 (Sirius-IDR2-(MS2)₈+MCP-Halotag+Halotag-JF549) and IDR3 (IDR3-U4+TO-bPNA) was readily established in U2OS cells by lipofectamine plasmid transfection and staining with dyes delivered in media (HaloTag-JF549, TO-bPNA). Live cell imaging revealed partially overlapped red (IDR2, HaloTag-JF549) and green (IDR3, TO-bPNA) foci (FIG. 9, top row), which was expected given the close proximity (~4.6 kB) of the two loci on chromosome 19. Exclusion of TO-bPNA from media resulted in loss of IDR3 foci, leaving only red IDR2 foci visible (FIG. 9, middle row). Additionally, identical plasmid transfection protocols were carried out in which the FLURIL gRNA was replaced with a CRISPRainbow gRNA bearing PP7 hairpins (Sirius-IDR3-8×PP7). When stained with HaloTag-JF549 and TO-bPNA, only red foci were visible (FIG. 8 bottom row), consistent with URIL-selective fluorogenic binding of the TO-bPNA probe previously observed in fixed cells (FIG. 5). FLURIL-tagged IDR3 loci was easily tracked over 13s without significant photobleaching or dye blinking, performing comparably to IDR2 loci tracking with the established CRISPR-Sirius (8X-MS2) system. Time-lapse imaging was carried out for 96 frames at a capture rate of 136 ms per frame, yielding IDR2 and IDR3 trajectories on identical and homologous chromosomes of similar range (~100-200 nm) but in different patterns of loci territories, consistent with labeling of proximal, but distinct loci (FIG. 10). FLURIL-tagging of gRNA thus gave a highly usable fluorescence signal-to-noise ratio with single locus labeling, likely due to minimal background fluorescence from unbound TO-bPNA, unlike constitutively fluorescent FP and HaloTag systems. Though single molecule fluorescence tracking has not been demonstrated, we note that the sole FLURIL-tag was sufficient for labeling the low-copy number IDR3 locus (45 copies over 1.5 KB). Under standard operating conditions, the low background of a single FLURIL gRNA tag affords 7× greater brightness (FIG. 13) than the CRISPR-Sirius gRNA tags that have a substantially larger molecular footprint of 8 bacteriophage RNA hairpins bound by bacteriophage coat protein fusions.

### DISCUSSION

Taken together, these data establish FLURIL-tagging as an efficient and convenient strategy for intracellular RNA, RNP, and DNA tracking that leverages selective intracellular triplex hybridization of cell-permeable bPNA to URILs with fluorogenic binding to establish a robust and bright RNA tracking signal. We find that TO-bPNA exhibits significant (~600×) enhancement of emission intensity upon bPNA triplex hybridization with structured RNAs URILs in vitro and a substantive increase in quantum yield from ~0.01% in the unbound state to 43% in the RNA complex. This significant fluorogenic response to URIL-RNAs translated to 5-10× intracellular signal-to-noise and a highly usable, low-background cellular imaging of URILs. This method requires a modest 4 bp stem site within an RNA of interest to install an 8 nt URIL and plasmid transfection to introduce the modified RNA. While an engineered biomolecule (URIL-RNA) is needed, this is a universal requirement for the most broadly used, genetically-encoded live cell tracking strategies.

Intracellular FLURIL-tagging of RNAs in both fixed and live cells was directly verified using established protein-labels from endogenous mammalian RNPs as well as bacteriophage MS2-labeling, which remains the gold standard for RNA tracking. In the present study, we have inserted stabilized hairpin structures to host the URIL site and included a PP7 hairpin as a control element in IDR3-U4; however, a URIL could be inserted into a native fold by replacement of an existing native 4 bp stem buttressed by other structures in the ROI, further streamlining the FLURIL-tag footprint. Indeed, RBP fusions with fluorescent proteins (FP) confirmed that FLURIL-tags were labeling the correct RNA species, while cells lacking the correct RNA-protein pairing resulted in segregation of FLURIL tag and RBP-FP fusion signals. Under live cell tracking conditions, FLURIL tags also performed comparably to established MS2-based methods (CRISPR-Sirius), while occupying a considerably more compact molecular footprint. It is particularly noteworthy that the FLURIL tag only requires replacement of a 4 bp stem with an 8 nt URIL to enable RNP live cell tracking with a cell-permeable, ~IkD bPNA probe.

Though there remain many appealing aspects to MS2 labeling including multiplex and multicolor approaches, the use of protein fusions and multiple RNA hairpin sites encumbers the RNA of interest with substantial steric bulk, adding 41-47 kD of mass per MCP-FP and MCP-HaloTag fusion, respectively. The steric bulk and RNA secondary structures required for protein labeling have raised concerns that this could affect native transcript processing and tertiary contacts;¹ however, inhibition of transcript degradation by insertion of bacteriophage (MS2/PP7) hairpins is less of an issue in mammalian cells. In contrast, FLURIL tags add negligible additional mass to the RNA target and a structural perturbation which may be as minor as replacement of a duplex stem with a triplex stem. Prior work has demonstrated that this replacement does not disrupt proximal RNA domains, retaining tertiary interactions as well as catalytic function. The FLURIL tag also avoids the use of labile G-quadruplex domains and insertion of foreign secondary structures found in some SELEX-derived dye-binding aptamers such as Spinach and Mango. Furthermore, as fluorogenic dye binding is driven by bPNA triplex hybridization to the URIL rather than direct RNA recognition of the dye, prosthetic groups may be used to decorate RNPs without the technical demand of a SELEX campaign. In particular, other fluorogens in the cyanine family could potentially be used in FLURIL tagging, thus expanding the color range available without laborious aptamer selection procedures. Analogously, the aptamer Pepper binds a family of fluorogen dyes with significant structural variation, suggesting that minimal specific contacts are needed to access fluorogenic binding.

The unique advantages of FLURIL tagging are counterbalanced by some limitations. A URIL motif must be installed in the RNA of interest, requiring non-native RNA expression by transfection. The efficiency of tracking as described herein thus depends on efficiency of transfection and vector design, which can lead to variability in labeling outcomes; additionally, transfection procedures yield elevated, non-native transcript levels. These issues may be alleviated with genome editing, though this comes with a significant cost and effort per experiment. Currently, FLURIL tagging has only been demonstrated with a single color, though efforts to expand both color selection and sequence scope are underway. Despite these caveats, this example demonstrates the utility of the FLURIL tag, as well as an appealing compatibility with popular methods such as MS2-labeling. FLURIL tagging can be carried out simultaneously with the widely-used MS2/MCP/HaloTag technology, and is likely also compatible with other methods that include dye aptamers, Riboglow, dCas13-gRNA, FISH. We anticipate RNA FLURIL tagging will be a useful tool that may be used in conjunction with other methods to facilitate RNA tracking.

### Materials and Methods

General Materials and Methods. All chemicals were used without further purification from commercial sources as indicated, unless otherwise noted. DNAs and RNAs were purchased from Integrated DNA Technologies (IDT). Nucleic acid strands shorter than 20 nt were used without further purification. Otherwise, the DNAs/RNAs were purified by TBE-urea denaturing gel. DNA stock solutions were serially diluted in MilliQ water and concentrations were determined by measuring solution absorbance at 260 nm on a Thermo Fisher Nanodrop 2000. Sample fluorescence was measured on a Thermo Fisher Nanodrop 3300. Cell lines were acquired from ATCC (HEK-293T, cat #CRL-2316; U2OS, cat #HTB-96) and cultured according to ATCC protocols. Microscopy images are shown in green and red color to correspond with green and red emission wavelengths as in green fluorescent protein (GFP), red fluorescent proteins (RFP, dTomato).

Nucleic acid sequences. RNAs for in vitro fluorescence studies shown below were purchased. All RNAs were annealed prior to use. Duplexes were annealed together from A and B strands indicated.

12-U4-12 A:
(SEQ ID NO. 1)
5′-CGCAUAGCUCAGUUUUGACUCGAUACGC-3′
12-U4-12 B:
(SEQ ID NO. 2)
5′-GCGUAUCGAGUCUUUUCUGAGCUAUGCG-3′
12-U6-12 A:
(SEQ ID NO. 3)
5′-CGCAUAGCUCAGUUUUUUGACUCGAUACGC-3′
12-U6-12 B:
(SEQ ID NO. 4)
5′-GCGUAUCGAGUCUUUUUUCUGAGCUAUGCG-3′
RNAI U6:
(SEQ ID NO. 5)
5′-GGCAGCUUUUUUUUGGUAGUUUUUUGCUGCC-3′
RNAII WT:
(SEQ ID NO. 6)
5′-GCACCGCUACCAACGGUGC-3′

The following RNA sequences were delivered into cells by plasmid transfection for intracellular labeling.

U4 tRNA:
(SEQ ID NO. 7)
5′-GCCCGGAUAGCUCAGUCGGUAGAGCAGCGGCCGUUUUCGCUC
CGGCGUUUUCGGCCGCGGGUCCAGGGUUCAAGUCCCUGUUCGGGCG
CCA-3′
U4-MS2 (MBSV5) tRNA:
(SEQ ID NO. 8)
5′-GCCCGGAUAGCUCAGUCGGUAGAGCAGCGGCCGUUUUCGCAC
AUGAGGAUCACCCAUGUGCGUUUUCGGCCGCGGGUCCAGGGU
UCAAGUCCCUGUUCGGGCGCCA-3′
NEG tRNA:
(SEQ ID NO. 9)
5′-GCCCGGAUAGCUCAGUCGGUAGAGCAGCGGCCGCGCGCGCUCC
GGCGCGCGCGGCCGCGGGUCCAGGGUUCAAGUCCCUGUUCGGGCGC
CA-3′
(GU)₈-U4 RNA:
(SEQ ID NO. 10)
5′-CGGCCGUUUUCGCUCCGGCGUUUUCGGCCGGUGUGUGUGUGUG
UGU-3′

HPLC measurements. Fluorogenic bPNA probe was purified using HPLC: Hitachi D-7000 (interface), Hitachi L-7150 (UV-detector) and Hitachi L-7400 (pump). Analytical and semi-preparative HPLC was carried out with C 18 reverse phase columns. HPLC solvent A: 99% MilliQ H₂O, 1% HPLC grade acetonitrile, 0.1% trifluoroacetic acid; HPLC solvent B: 10% MilliQ H₂O, 90% HPLC grade acetonitrile, 0.07% trifluoroacetic acid.

In vitro fluorescence measurements. The nucleic acids were annealed from 95° C. to pre-form the structure before use. RNAs were annealed with bPNA at 1:1 ratio and incubated for 30 min following slow cooling (50 mM HEPES, pH 7.5, 100 mM NaCl, 2 μM RNA, 2 μM bPNA). Sample fluorescence was measured using a Thermo Fisher Nanodrop 3300 (relative fluorescence units (RFU), excitation=470 nm, emission=522 nm, 2 μL sample volume). All fluorescence values are the average of triplicate measurements, starting from fresh sample preparation. Error bars indicate standard deviation. Data processed using OriginPro and Graphpad Prism. All fluorescence values are the average of triplicate measurements, starting from fresh sample preparation. Error bars indicate standard deviation.

Fitting methodology for binding curves. Probes (bPNA-TO) were held at a constant concentration of 100 nM and treated with RNA to final concentrations from 0 to 1.4 μM in buffer (50 mM HEPES, pH 7.5, 100 mM NaCl). The experiments were triplicated from sample preparation and error bars indicate standard deviation. The data were fit with the following equation to obtain dissociation constant Kd:

$$RFU = \frac{m * ( Kd + x + 100 ) - m * sqrt [ sqr ( Kd + x + 100 ) - 4 * x * 100 ]}{2}$$

Quantum yield measurements. Relative quantum yield was calculated against fluorescein (quantum yield=92%). All dye samples (TO, TO-bPNA) were prepared at 1 μM with hybrid samples prepared at 1:1 TO:RNA ratio (50 mM HEPES, pH 7.5, 100 mM NaCl). Sample concentrations were determined by UV absorbance (Cary UV-Vis-NIR Spectrophotometer) and adjusted to obtain matched sample absorbances, not to exceed 0.04 at 470 nm (+0.0002). The fluorescence was measured by Quantamaster 8000 spectrofluorometer and corrected for anisotropy with a vertical polarizer in the excitation path (470 nm) and a polarizer in the emission path (507 nm) set at the magic angle (54.7°), with excitation and emission slit widths=5 nm). Integrated emission of the bPNA hybrid was 49.7% that of fluorescein, indicating a relative quantum yield of 43%.

Construction of RNA plasmid vector. The DNA inserts corresponding to modified tRNA scaffolds (U4, U4-MS2, NEG (RNA) were annealed into duplexes from which 2 μg were digested with SalI and XbaI (Thermofisher, 2× Tango buffer, 12 h), following Thermofisher protocol. Vector pAV U6+27 (1 μg) was separately digested with SalI and XbaI (12 h) and all digested products were gel purified (band isolation from with Qiagen QIAquick Gel Extraction Kit). Purified products were ligated (DNA insert: linearized vector=5:1) with T4 DNA ligase (Thermofisher), following published protocol. Ligation product was transformed into DHSα for amplification and the resulting mixture was inoculated on agar plate with ampicillin selection. After 16 hours, several colonies were picked and amplified in LB media containing ampicillin. Harvested plasmid was isolated by miniprep (Qiagen) and verified by sequencing. Tomato-TDP43 and MCP-TagRFPt plasmids were obtained from Addgene (#28205 and #64541), and amplified in the corresponding E. coli cell lines.

Cell treatment (fixed cell analysis). HEK-293T cells were cultured based on ATCC protocol. Cells were seeded to 35 mm culture dish (Thermofisher) with clean coverslip (Thermofisher) attached to the bottom at the concentration of 5×10⁵/ml. After one day of incubation, the cells were transfected with corresponding plasmids (1000 ng each per dish) with Lipofectamine 3000 (Invitrogen) following the online protocol and the cells were incubated for 24 hr. Then the culture medium was removed and the fresh medium containing 1 μM bPNA-dye was added to the attached cells. The cells were incubated at 37° C. for 2-8 hr and the medium was discarded. Hoechst 33258 (Invitrogen) in culture medium was added at the final concentration of 200 ng/ml to the cells and incubated for 15 min at 37° C. and the medium was discarded. The cells were washed with PBS, fixed with 4% formaldehyde in PBS for 15 min at room temperature and rinsed with PBS. The coverslip was transferred to glass slides for fluorescence microscopy. Cells were imaged under Olympus FV3000 systems (Objective lens: 40×, Zoom: 1×. Blue channel: (excitation) 405 nm (emission) 422 nm, Voltage: 355 V, detection range: 420-470 nm; Green (excitation) 488 nm, (emission) 520 nm, Voltage: 580 V, detection range: 499-599 nm). Three or more replicate data sets were obtained that showed consistent findings, starting from cell seeding.

Cellular fluorescence quantification (fixed cell). Using ImageJ software, the fluorescence intensity in a circular area of diameter ~5 μm within a single cell (HEK-293) was selected and the grayscale was reported. For each treatment, 10 areas were selected in this manner from 10 different cells. The intensity from NEG-tRNA plasmid treated cells was normalized to 1.0.

Plasmid Construction. The dCas9 (Addgene #121936) and MCP-HaloTag (Addgene #121937) plasmids were used without modification and expression vector for the guide RNAs (gRNAs) were modified from previous gRNA plasmids. The single gRNA plasmid for IDR3-U4 gRNA was constructed from pLH-sgRNA1-2×PP7 (Addgene #75390) where PP7-U4 was inserted to replace 2×PP7 (FIG. 11). The dual gRNA plasmid 9 was constructed from pPUR-hU6-Sirius-8×MS2-mU6-Sirius-8×PP7 (Addgene #121944), in which CMV—HH-PP7-4USL-HDV was inserted to replace mU6-Sirius-8×PP7, resulting in pPUR-hU6-Sirius-8×MS2-CMV—HH-PP7-4USL-HDV. The HH and HDV cassettes were inserted for precise gRNA sequences through a cleavage mechanism at a defined position expressed under a pol II promoter in eukaryotic cells. The targeting sequences for IDR2 and IDR3 were 5′GGAGAGGCTGGG-3′ (SEQ ID NO. 11) and 5′-AGCAGATGTAGG-3′ (SEQ ID NO. 12), respectively. The gRNA sequences were ordered from IDT and inserted using BbsI restriction enzyme following the same protocol.

Cell Culture, Lentivirus Production and Transduction (live cell experiments). We cultured human osteosarcoma U2OS at 37° C. in Dulbecco-modified Eagle's Minimum Essential Medium (DMEM) containing high glucose and supplemented with 10% (vol/vol) fetal bovine serum. U2OS^{dCas9-HSA/MCP-HaloTag} cell line that was generated by lentiviral transduction and sorted by FACSFusion cell sorter (BD Bioscience). Lentiviral particles carrying dCas9 and MCP-HaloTag were generated by HEK-293 cells using the same protocol. For lentiviral transduction, U2OS cells maintained as described above were transduced by Spinfection in 6-well plates with lentiviral supernatant for 48 hours and ~2×10⁵ cells were combined with 1 ml lentiviral supernatant and centrifuged for 30 minutes at 1200×g. For imaging, U2OS^{dCas9-HSA/MCP-HaloTag} cells were grown on 35-mm glass-bottom dishes (MatTek) and 2 μg of sgRNA plasmid were transfected using TransIT transfection reagent (Mirus) following manufacturer's protocol. Cells were washed with fresh media 24 hours post-transfection and imaged after another 24 h incubation. A final concentration of 1 μM TO-bPNA was added to the culture medium two hours before imaging. Cell toxicity was not observed even with prolonged incubation (up to 48 hours) in the presence of TO-bPNA. Cells were tested and found to be free of mycoplasma contamination.

Flow cytometry. The cell line U2OS^{dCas9-HISA/MCP-Halo Tag} was generated using the same protocol that generated U2OS^{dCas9-HAS/PCP-GFP/MCP-HaloTag} with the following modifications: (1) The PCP-GFP for labeling PP7 stem loop was not added; (2) cells expressing the dCas9-p2A-HSA and MCP-HaloTag (stained with HaloTag-JF549) were selected using a BD FACSAria Fusion cell sorter (BD Bioscience) equipped with 405, 488, 561 and 640 nm excitation lasers and standard emission filters for PE (582/15) and APC (670/30); (3) AlexaFluor 647-conjugated anti-mouse CD24 antibody (BioLegend) was used to stain for HSA (heat stable antigen, mouse) carried on the dCas9 plasmid; (4) U2OS^{dCas9-HSA/MCP-HaloTag} was not selected from a single cell. FACS sorting for dCas9 positive cells was carried out following sample staining (1 μL Alexa Fluor-647 conjugated anti-mouse CD24 antibody, 100 μL cell solution, 30 min). FACS sorting of MCP-HaloTag positive cells was carried out after staining with HaloTag-JF549 (2 nM dye, 12-24 hr).

Fluorescence Microscopy (live cell imaging). Cell imaging (U2OS) was carried out on an Olympus IX83 microscope equipped with three EMCCD cameras (Andor iXon 897) mounted on a 4-camera splitter, four lasers (405 nm, 488 nm, 561 nm, and 647 nm), mounted with a 1.6× magnification adapter and 60× apochromatic oil objective lens (NA 1.5), resulting in a total of 96× magnification. The microscope stage incubation chamber was maintained at 37° C. with CO₂ and humidity supplement. A laser quad-band filter set for TIRF (emission filters at 445/58, 525/50, 595/44, 706/95) was used to collect fluorescence signals simultaneously. Data acquisition was carried out with CellSens 4.1.1 software. Localization precision was ~5 nm in 4 seconds, ~6 nm in 16 seconds, and ~10 nm in 80 seconds. The video was recorded 136 ms per frame with a total of 96 frames and 100 ms exposure time. Image size was adjusted to show individual nuclei and intensity thresholds were set on the basis of the ratios between nuclear foci signals to background nucleoplasmic fluorescence.

Image Processing. The images were registered and analyzed by Fij and Mathematica (Wolfram) software. To achieve subpixel registration accuracy, parameters for shifting, scaling, and rotating camera images were determined by the least-squares fitting of fluorescent bead images (100 nm TetraSpeck fluorescent microspheres, Invitrogen). The experimental data from each channel were processed through an affine transformation and overlapped in false-color channels for visualization. The locus trajectory was obtained by the tracking of locus position over time and graphs were generated by OriginPro (OriginLab version 2019b).

### Synthetic Procedures

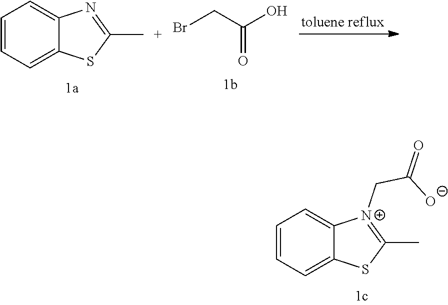

Synthesis of 2-(2-Methylbenzo[d]thiazol-3-ium-3-yl)acetate (1c). A mixture of 2-methylbenzothiazole (1a, 4.78 g, 32 mmol, 1.0 eq.) and bromoacetic acid (1b, 6.5 g, 47 mmol, 1.5 eq.) in toluene (100 ml) was heated at reflux overnight. After cooling to room temperature, the resulting mixture was filtered and the solid was washed with toluene (3×5 ml) to afford the product (1c, 6.5 g, 98%) after drying.

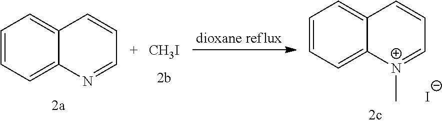

Synthesis of 1-methylquinolin-1-ium iodide (2c). A mixture of quinoline (2a, 2 g, 15.5 mmol, 1.0 eq.) and iodomethane (2b, 2 ml, 32.2 mmol, 2.1 eq.) in 1,4-dioxane (150 ml) was heated at reflux for 1 hr. After cooling to room temperature, the resulting mixture was filtered and the solid was washed with diethyl ether (3×5 ml) and hexanes (3×5 ml), affording the product (2c, 4 g, 95%) after drying.

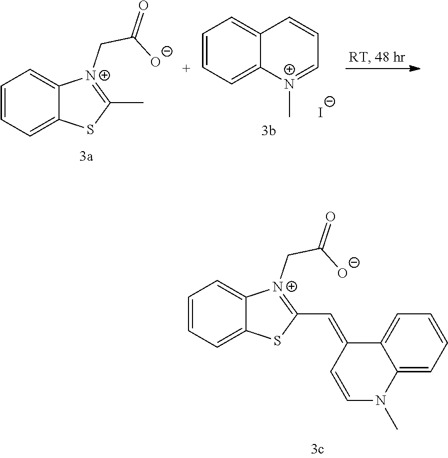 [SMILES: Cc1sc2ccccc2[n+]1CC(=O)[O-] ; C[n+]1cccc2c1=C[C+]=CC=2 ; [I-] ; CN1C=C/C(=C\c2sc3ccccc3[n+]2CC(=O)[O-])c2ccccc21]

Synthesis of TO-acetate (3c). 2-(2-Methylbenzo[d]thiazol-3-ium-3-yl)acetate (3a, 2.14 g, 10.4 mmol, 1.4 eq.), 1-methylquinolin-1-ium iodide (3b, 2 g, 7.4 mmol, 1 eq.) and triethylamine (2.24 g, 22.2 mmol, 3 eq.) were added in a round bottom flask with 30 ml dichloromethane. The mixture was stirred at room temperature for 48 hr. After 48 hr, the mixture was concentrated and 16 ml ethanol and 4 ml diethyl ether were added. The resulting mixture was stirred at 4° C. overnight. The resulting precipitate was collected by filtration and washed with diethyl ether (3×8 ml). The solid was then dissolved in 12 ml methanol with 3 ml water and was stirred overnight at 4° C. The resulting precipitate was collected by filtration and washed with water (3×8 ml). Then the solid was resuspended in acetone and stirred at room temperature for 1 hr. The final product was obtained by filtration and was washed with acetone (3×8 ml) and dried in vacuum to afford TO-acetate as red solid (3c, 160 mg, 6.4%). ¹H NMR (DMSO-d₆): 8.54 (1H, d); 8.48 (1H, d); 7.98 (1H, d); 7.94 (1H, d); 7.86 (1H, t); 7.64 (1H, d); 7.55 (2H, m); 7.38 (1H, t); 7.18 (1H, d); 6.77 (1H, s); 5.09 (2H, s); 4.10 (3H, s). HRMS (ESI): Mass calculated: [M+H⁺]=349.1005; Mass found: [M+H⁺]=349.1004.

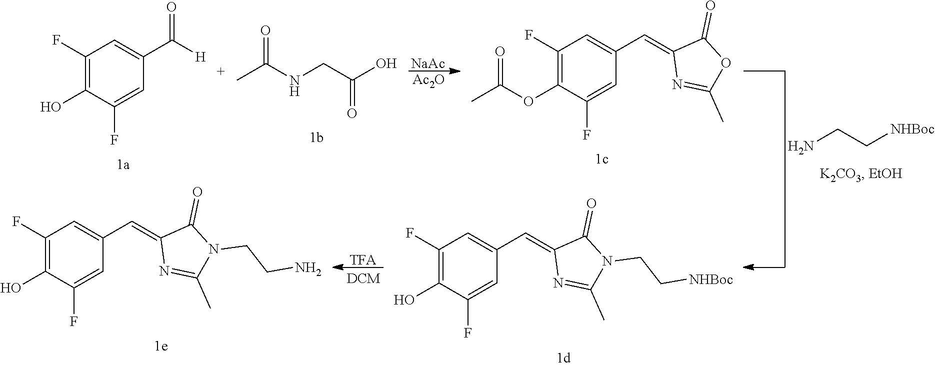

Synthesis of (Z)-2,6-difluoro-4-((2-methyl-5-oxo oxazol-4 (5H)-ylidene)methyl) phenyl acetate (1c). N-Acetylglycine (1b, 0.22 g, 1.9 mmol), anhydrous sodium acetate (0.156 g, 1.9 mmol), 4-hydroxy-3,5-difluorobenzaldehyde (1a, 0.3 g, 1.9 mmol), and acetic anhydride (0.72 ml) were stirred at 100° C. for 2 h. Then the reaction was cooled to room temperature and 3 ml ethanol was added to the mixture. The mixture was stirred at 4° C. overnight. The resulting solid was collected by filtration, washed with cold ethanol, hot water, hexanes and dried to afford 0.32 g (60%) of product as a yellow solid.

Synthesis of DFHIBI-ethylenediamine (1e). (Z)-2,6-difluoro-4-((2-methyl-5-oxoxazol-4 (5H)-ylidene)methyl) phenyl acetate (1c, 100 mg, 0.36 mmol), Boc-ethylenediamine (110 mg, 0.69 mmol) and potassium carbonate (0.13 g) were added to 2 ml ethanol and refluxed for 4 hr. The mixture was cooled to room temperature and the solvent was removed in vacuum. The residue was redissolved in acetate buffer (pH=3.0) and ethyl acetate (1:1 mixture). The organic layer was collected and the solvent was removed in vacuum. The residue was purified by column (DCM:MeOH=10:1), yielding 104 mg (76%) product (1d) as yellow solid. The product was added to TFA:DCM=1:1 for Boc deprotection.

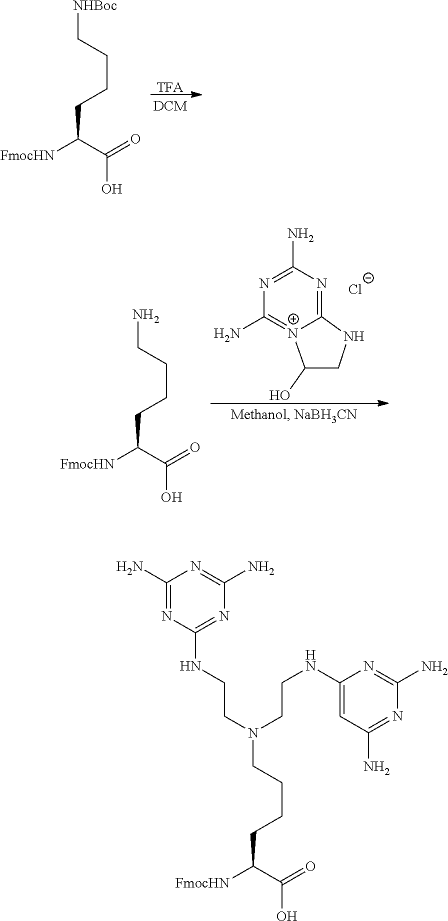

Synthesis of Fmoc-K^{2M}—OH. Fmoc-Lys (Boc)-OH (10 g, 21 mmol) was dissolved in 100 mL dichloromethane and 50 mL of trifluoroacetic acid was added. The reaction was stirred for 1 h and the solution was condensed to syrup under a stream of N2. Dichloromethane (50 mL) was added to dissolve the syrup and was removed by a stream of N2. The syrup was dissolved in 200 mL methanol and the pH was adjusted to 5 with solid NaHCO₃. Melamine aldehyde (Hemiaminal form, 9.5 g, 46.2 mmol) and NaBH₃CN (2.93 g, 46.2 mmol) was aliquoted into 4 portions, respectively. To the reaction solution, 2 portions of melamine aldehyde were added and were stirred and incubated at 50° C. for 30 min. Then the reaction was taken out to cool to room temperature and 1 portion of NaBH₃CN was added. The reaction was stirred at room temperature for another 30 min. Then 1 portion of aldehyde was added, incubated at 50° C. for 30 min and 1 portion of NaBH₃CN was added and incubated at room temperature for 30 min. These addition of aldehyde and reductant steps were repeated until all 4 portions of aldehyde and 3 portions of NaBH₃CN were added. The last portion of NaBH₃CN was added to the reaction and stirred for 30 min at room temperature. The reaction was monitored by HPLC. The remaining monoadduct (Fmoc-KIM-OH) was reacted by adding 2.3 g of melamine aldehyde, incubating at 50° C. for 30 min and 0.72 g NaBH₃CN was added to finish the reaction. Methanol was reduced to ~50 ml and was discarded after centrifugation, yielding white solid as crude product after drying. The reaction was quenched by adding 10 mL IN hydrochloric acid and the solid was triturated. The hydrochloric acid was discarded after centrifugation. Acetone (30 mL) was added to the residue and the residue was triturated. The acetone was removed by centrifugation. The acetone wash was repeated 3 times and ethanol was added for trituration instead of acetone. After removing ethanol by centrifugation, the product (~11 g, 78%) was obtained as white solid.

Synthesis of Fmoc-αK^{2M}—OH. Boc-Lys(Fmoc)-OH (1 g, 2.1 mmol) was dissolved in 20 mL dichloromethane and 4 mL of trifluoroacetic acid was added. The reaction was stirred for 1 h and the solution was condensed to syrup under a stream of N2. Dichloromethane (10 mL) was added to dissolve the syrup and was removed by a stream of N2. The syrup was dissolved in 30 mL methanol and the pH was adjusted to 5 with solid NaHCO₃. Melamine aldehyde (0.95 g, 4.62 mmol) and NaBH₃CN (0.293 g, 4.62 mmol) were aliquoted into 4 portions, respectively. To the reaction solution, 2 portions of melamine aldehyde were added and incubated at 50° C. for 30 min with stirring. Then the reaction was taken out to cool to room temperature and 1 portion of NaBH₃CN was added. The reaction was stirred at room temperature for another 40 min. Then 1 portion of aldehyde was added, incubated at 50° C. for 30 min and 1 portion of NaBH₃CN was added and incubated at room temperature for 40 min. These additions of aldehyde and reductant steps were repeated until all 4 portions of aldehyde and 3 portions of NaBH₃CN were added. The last portion of NaBH₃CN was added to the reaction and stirred for 40 min at room temperature. The reaction was monitored by HPLC. The remaining monoadduct (Fmoc-αK^{M}—OH) was reacted by adding 0.25 g of melamine aldehyde, incubating at 50° C. for 30 min and 0.078 g NaBH₃CN was added to finish the reaction. Methanol was reduced to 5 ml and was discarded after centrifugation, yielding pale yellow solid as crude product after drying. The reaction was quenched by adding 2 mL IN hydrochloric acid and the solid was triturated. The hydrochloric acid was discarded after centrifugation. Acetone (5 mL) was added to the residue and the residue was triturated. The acetone was removed by centrifugation. The acetone wash was repeated 3 times and ethanol was added for trituration instead of acetone. After removing ethanol by centrifugation, the product (0.85 g, 60%) was obtained as pale yellow solid. ¹H NMR (400 MHz, DMSO-d₆): 7.91 (2H, d); 7.79 (10H, d); 7.66 (2H, d); 7.41 (2H, t); 7.32 (2H, t); 7.25 (1H, t); 4.28 (2H, d); 4.20 (1H, t); 3.33 (4H, q); 3.28 (1H, m); 2.95 (4H, q); 2.76 (2H, q); 1.62 (2H, q); 1.15-1.50 (4H, m). ¹³C NMR (100 MHz, DMSO): 174.3; 170.7; 166.3; 154.5; 144.3; 141.1; 128.0; 127.5; 125.5; 120.5; 80.1; 79.6; 65.5; 50.6; 49.0; 47.2; 29.3; 23.6; 21.9. HRMS (ESI): calculated for [M+H⁺]=673.3430, [M+2H]=337.1751, found [M+H]⁺=673.3425, [M+2H]=337.1756

Solid phase peptide synthesis. Peptide synthesis was performed manually using Rink Amide resin (100-200 mesh, loading 0.3 mmol/g) employing standard Fmoc chemistry. With 150 mg resin, 0.25 M of Amino acids were coupled with 0.25 M of PyAOP and 0.25 M DIPEA in 2 ml NMP. Fluorogenic dyes were coupled using three equivalents of dye, 3.3 equivalents of HBTU, and 3.3 equivalents of DIPEA in 2 ml DMF. Fmoc cleavage was performed with 2 mL of piperidine:NMP (1:1) with 3% DBU. Dye and coupling reagents were allowed to react for 15 min before addition to resin. For TO peptides, TO-acetate was coupled to the peptide N-terminus directly. For DFHBI peptide, the peptide was reacted with 3 equivalents of succinic anhydride, and 3 equivalents of DIPEA for 15 min, washed and DFHBI-NH 2 was coupled to the N-terminus of the peptide. Peptides were cleaved from the solid support using 95% trifluoroacetic acid (TFA) and 5% H₂O for 2 h. Cold diethyl ether (Et₂O) was added to precipitate the peptide and the crude pellet was washed with cold Et₂O two times and dried over vacuum. Crude peptides were then dissolved in solvent A and purified by HPLC on a semi-prep C 18 reversed phase column at 8 mL/min. The UV detector was set at 238 nm. The purified peptides were lyophilized to dryness. The identity of peptide was checked by MALDI-TOF and purity checked by analytical HPLC on a C 18 column. (solvent A=0.1% TFA in water, solvent B=0.07% TFA in 90% acetonitrile, 10% water).

### REFERENCES

1 Braselmann, E., Rathbun, C., Richards, E. M. & Palmer, A. E. Illuminating RNA Biology: Tools for Imaging RNA in Live Mammalian Cells. Cell Chem Biol 27, 891-903 (2020)
2. Alexander, S. C., Busby, K. N., Cole, C. M., Zhou, C. Y. & Devaraj, N. K. Site-Specific Covalent Labeling of RNA by Enzymatic Transglycosylation. J. Am. Chem. Soc. 137, 12756-12759 (2015).
3. Jahn, K. et al. Site-specific chemical labeling of long RNA molecules. Bioconjug. Chem. 22, 95-100 (2011).
4. Smith, G. J., Sosnick, T. R., Scherer, N. F. & Pan, T. Efficient fluorescence labeling of a large RNA through oligonucleotide hybridization. RNA 11, 234-239 (2005).
5. Schmitz, A. G., Zelger-Paulus, S., Gasser, G. & Sigel, R. K. O. Strategy for Internal Labeling of Large RNAs with Minimal Perturbation by Using Fluorescent PNA. Chembiochem 16, 1302-1306 (2015).
6. Mao, S., Ying, Y., Wu, R. & Chen, A. K. Recent Advances in the Molecular Beacon Technology for Live-Cell Single-Molecule Imaging. iScience 23, 101801 (2020).
7. Paige, J. S., Wu, K. Y. & Jaffrey, S. R. RNA Mimics of Green Fluorescent Protein. Science 333, 642-646 (2011).
8. Filonov, G. S., Moon, J. D., Svensen, N. & Jaffrey, S. R. Broccoli: Rapid Selection of an RNA Mimic of Green Fluorescent Protein by Fluorescence-Based Selection and Directed Evolution. J. Am. Chem. Soc. 136, 16299-16308 (2014).
9. Warner, K. D. et al. A homodimer interface without base pairs in an RNA mimic of red fluorescent protein. Nat. Chem. Biol. 13, 1195-1201 (2017).
10. Dolgosheina, E. V. et al. RNA Mango Aptamer-Fluorophore: A Bright, High-Affinity Complex for RNA Labeling and Tracking. ACS Chem. Biol. 9, 2412-2420 (2014).
11. Chen, X. et al. Visualizing RNA dynamics in live cells with bright and stable fluorescent RNAs. Nat. Biotechnol. 37, 1287-1293 (2019).
12. George, L., Indig, F. E., Abdelmohsen, K. & Gorospe, M. Intracellular RNA-tracking methods. Open Biol. 8, (2018).
13. Schmidt, A., Gao, G., Little, S. R., Jalihal, A. P. & Walter, N. G. Following the messenger: Recent innovations in live cell single molecule fluorescence imaging. Wiley Interdiscip. Rev. RNA 11, e1587 (2020).
14. Sunbul, M. & Jäschke, A. SRB-2: a promiscuous rainbow aptamer for live-cell RNA imaging. Nucleic Acids Res. 46, e110 (2018).
15. Bouhedda, F. et al. A dimerization-based fluorogenic dye-aptamer module for RNA imaging in live cells. Nat. Chem. Biol. 16, 69-76 (2020).
16. Braselmann, E. et al. A multicolor riboswitch-based platform for imaging of RNA in live mammalian cells. Nat. Chem. Biol. 14, 964-971 (2018).
17. Trachman, R. J. & Ferré-D'Amaré, A. R. Tracking RNA with light: selection, structure, and design of fluorescence turn-on RNA aptamers. Q. Rev. Biophys. 52, e8 (2019).
18. Warner, K. D. et al. Structural basis for activity of highly efficient RNA mimics of green fluorescent protein. Nat. Struct. Mol. Biol. 21, 658-663 (2014).
19. Huang, H. et al. A G-quadruplex-containing RNA activates fluorescence in a GFP-like fluorophore. Nat. Chem. Biol. 10, 686-691 (2014).

20. Trachman, R. J., III et al. Structural basis for high-affinity fluorophore binding and activation by RNA Mango. Nat. Chem. Biol. 13, 807-813 (2017).
21. Guo, J. U. & Bartel, D. P. RNA G-quadruplexes are globally unfolded in eukaryotic cells and depleted in bacteria. Science 353, (2016).
22. Tutucci, E. et al. An improved MS2 system for accurate reporting of the mRNA life cycle. Nat. Methods 15, 81-89 (2018).
23. Bertrand, E. et al. Localization of ASH1 mRNA particles in living yeast. Mol. Cell 2, 437-445 (1998).
24. Grimm, J. B. et al. A general method to improve fluorophores for live-cell and single-molecule microscopy. Nat. Methods 12, 244-50, 3 p following 250 (2015).
25. Hoelzel, C. A. & Zhang, X. Visualizing and manipulating biological processes by using HaloTag and SNAP-tag technologies. Chembiochem 21, 1935-1946 (2020).
26. Garcia, J. F. & Parker, R. MS2 coat proteins bound to yeast mRNAs block 5′ to 3′ degradation and trap mRNA decay products: implications for the localization of mRNAs by MS2-MCP system. RNA 21, 1393-1395 (2015).
27. Garcia, J. F. & Parker, R. Ubiquitous accumulation of 3′ mRNA decay fragments in Saccharomyces cerevisiae mRNAs with chromosomally integrated MS2 arrays. RNA 22, 657-659 (2016).
28. Heinrich, S., Sidler, C. L., Azzalin, C. M. & Weis, K. Stem-loop RNA labeling can affect nuclear and cytoplasmic mRNA processing. RNA 23, 134-141 (2017).
29 Pickar-Oliver, A. & Gersbach, C. A. The next generation of CRISPR-Cas technologies and applications. Nat. Rev. Mol. Cell Biol. 20, 490-507 (2019).
30. Nelles, D. A. et al. Programmable RNA Tracking in Live Cells with CRISPR/Cas9. Cell 165, 488-496 (2016).
31. Chen, B. et al. Dynamic imaging of genomic loci in living human cells by an optimized CRISPR/Cas system. Cell 155, 1479-1491 (2013).
32. Lange, R. F. M. et al. Crystal Engineering of Melamine-Imide Complexes; Tuning the Stoichiometry by Steric Hindrance of the Imide Carbonyl Groups. Angew. Chem. Int. Ed Engl. 36, 969-971 (1997).
33. Whitesides, G. M. et al. Noncovalent Synthesis: Using Physical-Organic Chemistry To Make Aggregates. Acc. Chem. Res. 28, 37-44 (1995).
34. Ma, M. & Bong, D. Determinants of cyanuric acid and melamine assembly in water. Langmuir 27, 8841-8853 (2011).
35. Ma, M. & Bong, D. Controlled Fusion of Synthetic Lipid Membrane Vesicles. Acc. Chem. Res. 46, 2988-2997 (2013).
36. Arambula, J. F., Ramisetty, S. R., Baranger, A. M. & Zimmerman, S. C. A simple ligand that selectively targets CUG trinucleotide repeats and inhibits MBNL protein binding. Proc. Natl. Acad. Sci. U.S.A 106, 16068-16073 (2009).
37. Sanjayan, G. J., Pedireddi, V. R. & Ganesh, K. N. Cyanuryl-PNA monomer: synthesis and crystal structure. Org. Lett. (2000).
38. Piao, X., Xia, X. & Bong, D. Bifacial Peptide Nucleic Acid Directs Cooperative Folding and Assembly of Binary, Ternary, and Quaternary DNA Complexes. Biochemistry 52, 6313-6323 (2013).
39. Zeng, Y., Pratumyot, Y., Piao, X. & Bong, D. Discrete assembly of synthetic peptide-DNA triplex structures from polyvalent melamine-thymine bifacial recognition. J. Am. Chem. Soc. 134, 832-835 (2012).
40. Miao, S. et al. Unnatural bases for recognition of noncoding nucleic acid interfaces. Biopolymers 112, e23399 (2021).
41. Liang, Y. et al. Screening of Minimalist Noncanonical Sites in Duplex DNA and RNA Reveals Context and Motif-Selective Binding by Fluorogenic Base Probes. Chemistry 28, e202103616 (2022).
42. Mittapalli, G. K. et al. Mapping the landscape of potentially primordial informational oligomers: oligodipeptides and oligopeptides tagged with triazines as recognition elements. Angew. Chem. Int. Ed. 46, 2470-2477 (2007).
43. Mittapalli, G. K. et al. Mapping the landscape of potentially primordial informational oligomers: oligodipeptides tagged with 2,4-disubstituted 5-aminopyrimidines as recognition elements. Angew. Chem. Int. Ed. 46, 2478-2484 (2007).
44 Ura, Y., Beierle, J. M., Leman, L. J., Orgel, L. E. & Ghadiri, M. R. Self-assembling sequence-adaptive peptide nucleic acids. Science 325, 73-77 (2009).
45. Miao, S. et al. Duplex Stem Replacement with bPNA+Triplex Hybrid Stems Enables Reporting on Tertiary Interactions of Internal RNA Domains. J. Am. Chem. Soc. 141, 9365-9372 (2019).
46. Mao, J., DeSantis, C. & Bong, D. Small Molecule Recognition Triggers Secondary and Tertiary Interactions in DNA Folding and Hammerhead Ribozyme Catalysis. J. Am. Chem. Soc. 139, 9815-9818 (2017).
47. Xia, X., Piao, X. & Bong, D. Bifacial peptide nucleic acid as an allosteric switch for aptamer and ribozyme function. J. Am. Chem. Soc. 136, 7265-7268 (2014).
48. Xia, X., Piao, X., Fredrick, K. & Bong, D. Bifacial PNA complexation inhibits enzymatic access to DNA and RNA. Chembiochem 15, 31-36 (2013).
49. Liang, Y., Miao, S., Mao, J., DeSantis, C. & Bong, D. Context-Sensitive Cleavage of Folded DNAs by Loop-Targeting bPNAs. Biochemistry 59, 2410-2418 (2020).
50. Miao, S. et al. Bifacial PNAs Destabilize MALATI by 3′ A-Tail Displacement from the U-Rich Internal Loop. ACS Chem. Biol. 16, 1600-1609 (2021).
51. Rundell, S., Munyaradzi, O. & Bong, D. Enhanced Triplex Hybridization of DNA and RNA via Syndiotactic Side Chain Presentation in Minimal bPNAs. Biochemistry 61, 85-91 (2022).
52. Klymchenko, A. S. Solvatochromic and Fluorogenic Dyes as Environment-Sensitive Probes: Design and Biological Applications. Acc. Chem. Res. 50, 366-375 (2017).
53. Mohammed, H. S., Delos Santos, J. O. & Armitage, B. A. Noncovalent binding and fluorogenic response of cyanine dyes to DNA homoquadruplex and PNA-DNA heteroquadruplex structures. Artif. DNA PNA XNA 2, 43-49 (2011).
54. Vilaivan, T. Fluorogenic PNA probes. Beilstein J. Org. Chem. 14, 253-281 (2018).
55. Köhler, O., Jarikote, D. V. & Seitz, O. Forced intercalation probes (FIT Probes): thiazole orange as a fluorescent base in peptide nucleic acids for homogeneous single-nucleotide-polymorphism detection. Chembiochem 6, 69-77 (2005).
56. Fechter, E. J., Olenyuk, B. & Dervan, P. B. Sequence-specific fluorescence detection of DNA by polyamide-thiazole orange conjugates. J. Am. Chem. Soc. 127, 16685-16691 (2005).
57. Chenoweth, D. M., Meier, J. L. & Dervan, P. B. Pyrrole-imidazole polyamides distinguish between double-helical DNA and RNA. Angew. Chem. Int. Ed Engl. 52, 415-418 (2013).
58. Munyaradzi, O., Rundell, S. & Bong, D. Impact of bPNA Backbone Structural Constraints and Composition on Triplex Hybridization with DNA. Chembiochem e202100707 (2022) doi: 10.1002/cbic.202100707.
59. Devari, S., Bhunia, D. & Bong, D. Synthesis of Bifacial Peptide Nucleic Acids with Diketopiperazine Backbones. Synlett 33, 965-968 (2022).
60. Liang, Y., Mao, J. & Bong, D. Chapter Eight-Synthetic bPNAs as allosteric triggers of hammerhead ribozyme catalysis. in Methods in Enzymology (ed. Hargrove, A. E.) vol. 623 151-175 (Academic Press, 2019).
61. Tutucci, E., Vera, M. & Singer, R. H. Single-mRNA detection in living S. cerevisiae using a re-engineered MS2 system. Nat. Protoc. 13, 2268-2296 (2018).
62. Bong, D. T., Clark, T. D., Granja, J. R. & Ghadiri, M. R. Self-Assembling Organic Nanotubes. Angew. Chem. Int. Ed. 40, 988-1011 (2001).
63. Ghadiri, M. R., Granja, J. R., Milligan, R. A., McRee, D. E. & Khazanovich, N. Self-assembling organic nanotubes based on a cyclic peptide architecture. Nature 366, 324-327 (1993).
64. Nygren, J., Svanvik, N. & Kubista, M. The interactions between the fluorescent dye thiazole orange and DNA. Biopolymers 46, 39-51 (1998).
65. Suss, O., Motiei, L. & Margulies, D. Broad Applications of Thiazole Orange in Fluorescent Sensing of Biomolecules and Ions. Molecules 26, (2021).
66. Ma, H. et al. Multiplexed labeling of genomic loci with dCas9 and engineered sgRNAs using CRISPRainbow. Nat. Biotechnol. 34, 528-530 (2016).
67. Piatkevich, K. D. & Verkhusha, V. V. Guide to red fluorescent proteins and 67. biosensors for flow cytometry. Methods Cell Biol. 102, 431-461 (2011).
68. Ma, X. R. et al. TDP-43 represses cryptic exon inclusion in the FTD-ALS gene UNC13A. Nature 603, 124-130 (2022).
69 Brown, A. L. et al. TDP-43 loss and ALS-risk SNPs drive mis-splicing and depletion of UNC13A. Nature 603, 131-137 (2022).
70. Lagier-Tourenne, C. & Cleveland, D. W. Rethinking ALS: the FUS about TDP-43. Cell 136, 1001-1004 (2009).
71. Suk, T. R. & Rousseaux, M. W. C. The role of TDP-43 mislocalization in amyotrophic lateral sclerosis. Mol. Neurodegener. 15, 45 (2020).
72. Kuo, P. H., Doudeva, L. G., Wang, Y. T., Shen, C. K. J. & Yuan, H. S. Structural insights into TDP-43 in nucleic-acid binding and domain interactions. Nucleic Acids Res. 37, 1799-1808 (2009).
73. Kuo, P. H., Chiang, C. H., Wang, Y. T., Doudeva, L. G. & Yuan, H. S. The crystal structure of TDP-43 RRM1-DNA complex reveals the specific recognition for UG- and TG-rich nucleic acids. Nucleic Acids Res. 42, 4712-4722 (2014).
74. Yang, C. et al. The C-terminal TDP-43 fragments have a high aggregation propensity and harm neurons by a dominant-negative mechanism. PLOS One 5, e15878 (2010).
75. Ma, H. et al. CRISPR-Cas9 nuclear dynamics and target recognition in living cells. J. Cell Biol. 214, 529-537 (2016).
76. Ma, H. et al. CRISPR-Sirius: RNA scaffolds for signal amplification in genome imaging. Nat. Methods 15, 928-931 (2018).
77. Nielsen, S., Yuzenkova, Y. & Zenkin, N. Mechanism of eukaryotic RNA polymerase III transcription termination. Science 340, 1577-1580 (2013).
78. Xu, L., Zhao, L., Gao, Y., Xu, J. & Han, R. Empower multiplex cell and tissue-specific CRISPR-mediated gene manipulation with self-cleaving ribozymes and tRNA. Nucleic Acids Res. 45, e28 (2017).
79. Brouwer, A. M. Standards for photoluminescence quantum yield measurements in solution (IUPAC Technical Report). J. Macromol. Sci. Part A Pure Appl. Chem. 83, 2213-2228 (2011).
80 Levitus, M. Tutorial: measurement of fluorescence spectra and determination of relative fluorescence quantum yields of transparent samples. Methods Appl Fluoresc 8, 033001 (2020).
81. Song, W. et al. Imaging RNA polymerase III transcription using a photostable RNA-fluorophore complex. Nat. Chem. Biol. 13, 1187-1194 (2017).
82. Halstead, J. M. et al. An RNA biosensor for imaging the first round of translation from single cells to living animals. Science (2015) doi: 10.1126/science.aaa3380.
83. Chung, Y. C., Bisht, M. & Tu, L. C. CRISPR-based multi-locus real-time tracking reveals single chromosome dynamics and compaction. bioRxiv 2022.02.01.478681 (2022) doi: 10.1101/2022.02.01.478681.
84. Schindelin, J. et al. Fiji: an open-source platform for biological-image analysis. Nat. Methods 9, 676-682 (2012).

### Example 2. Screening of Minimalist Noncanonical Sites in Duplex DNA and RNA Reveals Context and Motif-Selective Binding by Fluorogenic Base Probes

### Summary

We hypothesize that programmable hybridization to noncanonical nucleic acid motifs may be achieved by macromolecular display of binders to individual noncanonical pairs (NCPs). As each recognition element may individually have weak binding to an NCP, we developed a semi-rational approach to detect low affinity interactions between selected nitrogenous bases and noncanonical sites in duplex DNA and RNA. A set of fluorogenic probes was synthesized by coupling abiotic (triazines, pyrimidines) and native RNA bases to thiazole orange (TO) dye. This probe library was screened against duplex nucleic acid substrates bearing single abasic, single NCP, and tandem NCP sites. Probe engagement with NCP sites was reported by 100-1000× fluorescence enhancement over background. Binding is strongly context-dependent, reflective of both molecular recognition and stability: less stable motifs are more likely to bind a synthetic probe. Further, DNA and RNA substrates exhibit entirely different abasic and single NCP binding profiles. While probe binding in the abasic and single NCP screens was monotonous, much richer binding profiles were observed with the screen of tandem NCP sites in RNA, in part due to increased steric accessibility. In addition to known binding interactions between the triazine melamine (M) and T/U sites, the NCP screens identified new targeting elements for pyrimidine-rich motifs in single NCPs and 2×2 internal bulges. We anticipate that semi-rational approaches of this type will lead to programmable noncanonical hybridization strategies at the macromolecular level.

### Background

Nucleic acid recognition at noncoding interfaces remains a frontier. Extensive studies have been performed on synthetic binders designed to target the minor and major groove interfaces of duplex DNA and RNA; in particular, the native and designed pyrrole-imidazole polyamide (Pyr-Im) systems established rules for sequence selectivity directed by hydrogen bond donor/acceptor matching in the minor groove. Similarly, functional and orthogonal synthetic base pairs may be prepared by shuffling Watson-Crick interfaces, yielding new pairs that are biophysically similar to native nucleic acids.

Recently, high throughput screening (HTS) of small molecule libraries have been applied to noncanonical RNA folds, in analogy to the approach for protein targets. Though selected designed systems and RNA HTS have yielded many important findings, general principles for targeting non-duplex secondary structures remain elusive. We hypothesized that a semi-rational approach akin to fragment-based strategies that includes both design and screening could be useful to gain insight into targeting of individual and tandem noncanonical pairs (NCPs) within internal bulges.

Noncanonical motifs are high-value targets due to their importance to noncoding function and notably present a richer set of available hydrogen bonding interfaces relative to duplex structures due to their unpaired or weakly paired nature. If it were possible to identify recognition elements for individual NCPs and minimal noncanonical motifs, this could enable programmable noncanonical hybridization to internal bulge structures when binders are displayed together on a macromolecular scaffold. This recognition problem has been solved for the TT/UU noncanonical pair: melamine (M) forms a base-triple with two T/U bases. Bifacial peptide nucleic acids (bPNAs) exploit this base triple to hybridize with oligo T/U internal bulges, forming hybrid stems that can be compatible or competitive with native nucleic acid function.

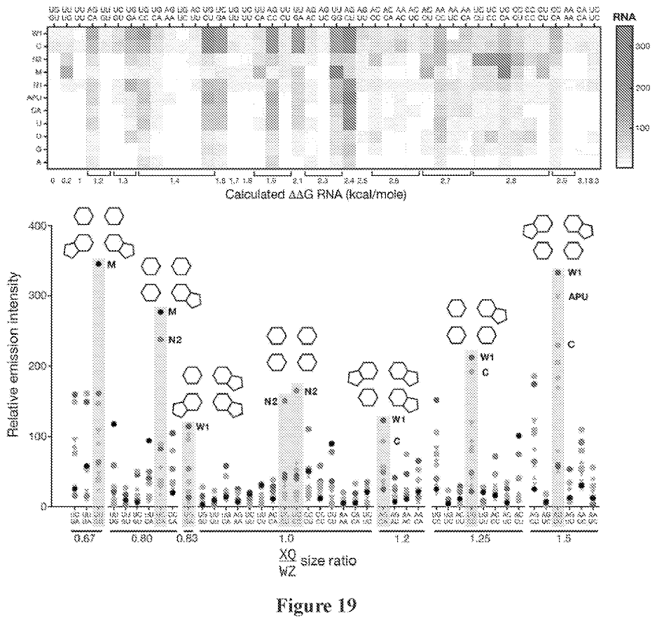

We speculate that the sequence scope of bPNA triplex hybridization could be expanded by the use of other synthetic and native bases that bind to NCPs other than TT/UU, independently or in conjunction with melamine. Notably, the melamine base itself only binds weakly to DNA, but can generate low nanomolar affinity binding when displayed multivalently on bPNAs, or micromolar affinity when coupled to an acridine intercalator. We have developed herein a fluorescence-based screen to facilitate the discovery of new NCP binding bases that is sufficiently sensitive to detect weak binding interactions, thus avoiding the higher synthetic cost of monomer and bPNA preparation. Synthetic and native bases were transformed into NCP probes by coupling to thiazole orange dye. The thiazole orange derivatives studied herein have low background affinity to dsDNA/RNA and low emission in solution, but we find that pendant nitrogenous bases with weak affinity to NCPs in duplex nucleic acids can trigger intercalation, resulting in increased emission. Similar sequence-directed “forced intercalation” and emission has been reported with TO-coupled Py-Im minor groove binders and PNAs. With these prior studies in mind, a TO-probe library was prepared with native RNA bases, symmetric triazines, and selected synthetic pyrimidines; each base has been shown— or has the potential—to form hydrogen bonding interactions with NCPs. In addition to the known native pairings and melamine triple with T/U, among this library is W1, an amino pseudoisocytidine which form a base-triple with T/C in DNA. Three classes of duplex substrates of increasing complexity were designed and tested: 1) single abasic sites, 2) single NCP sites, 3) tandem NCP sites. Single NCP (1×1 internal bulge) libraries in DNA and RNA were prepared with all 12 non Watson-Crick (WC) pairs. The larger space of tandem NCPs was scanned using a dsRNA library of 43 unique tandem NCPs (2×2 internal bulge); though not comprehensive, this set provides good coverage of purine/pyrimidine usage, excluding WC pairs. Each group was designed to reveal the step-wise impact of changing context on molecular recognition and point the way to a general design strategy for binding NCPs. Importantly, probe screens identified the important role of NCP stability in the inherent probe-reactivity of particular bulge sequences and highlighted stark differences in DNA vs RNA binding. Further, purine probes and adenine-containing sites were conspicuously unreactive, suggesting the sensitivity of this approach to steric demand. Though the context-dependent nature of NCP targeting complicates a difficult problem, the RNA-substrate driven binding trends revealed herein indicate that simple calculations of fold energetics can be useful when choosing an RNA target (FIG. 19). Most importantly, the screens have identified ammeline, W1 and cytidine as promising recognition elements for RNA NCP-targeting that could work in conjunction with the known U-site targeting function of melamine.

### Results and Discussion

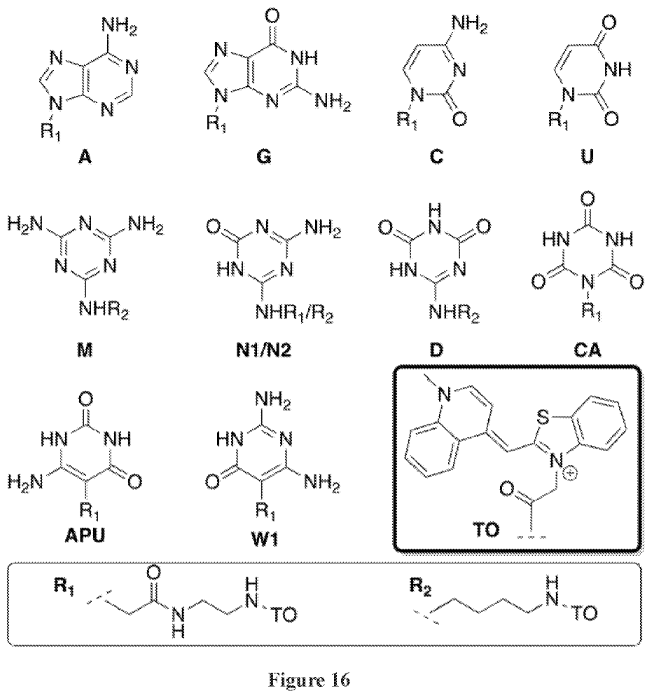

Probe synthesis. We synthesized a library of 11 NCP thiazole orange (TO) probes bearing adenine (A), guanine (G), cytidine (C), uridine (U), melamine (M), ammeline (N), ammelide (D), cyanuric acid (CA), 6-aminopseudouridine (APU), 6-aminopseudoisocytidine (W1) and two linker variations (R1, R2) (FIG. 16). The 4 native bases (A, G, C, U), cyanuric acid (CA), and the synthetic pyrimidines (APU, W1) were alkylated directly to form the acetate, then amidated with Boc-ethylenediamine. The symmetric triazine probes (M, N, D) were all prepared from alkylation of mono-Boc 1,4-diaminobutane with cyanuric chloride, followed by partial reaction with ammonium hydroxide or hydrolysis of the remaining chloride sites. Following Boc cleavage, the amines were acylated with thiazole orange to yield the R1 (A, G, C, U, APU, W1, CA) or R2 (M, N, D) linker family of probes; R1 and R2 linkers are identical in length (FIG. 16). An ammeline probe with the R1 linker (N1) was also prepared to examine linker effects. All probes exhibited negligible emission in solution without RNA.

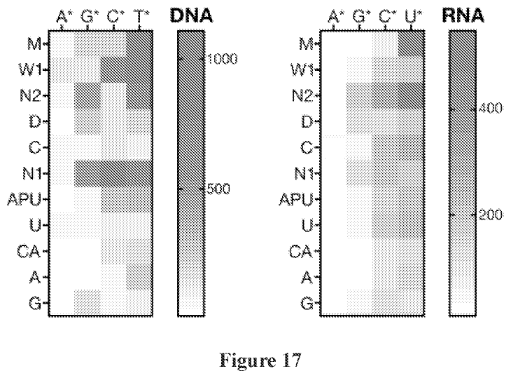

Abasic sites. Single T and C abasic sites in DNA exhibited the expected binding with complementary A and G probes, albeit with modest signals (FIG. 17). Much stronger binding signals were observed with M and W1 to T-abasic and T/C-abasic sites, respectively, confirming prior selectivity studies on these synthetic bases. Among the synthetic bases not previously examined, APU showed weak binding to T/C abasic sites and D showed a modest binding to G-abasic; all binding patterns can be rationalized through complementary hydrogen bonding patterns. Of the new bases, ammeline showed the strongest binding as well as a significant linker effect, with N₂ binding to G and C abasic sites while N1 bound to G, C and T abasic. Though N and W1 have tautomers with the same hydrogen bonding patterns, their binding profiles are distinct, even when the same linker (R1) is used for both (N1, W1). Notably, the A-abasic site did not accept any probe, and pyrimidine-abasic sites were overall much more probe-reactive. Remarkably, though there were general similarities, RNA binding profiles were significantly different to those in DNA. Like in DNA, A-abasic sites refused all probes and strong M, N selectivity for U/C abasic sites was observed. However, other selectivity patterns were diminished and blurred: while W1 elicited the most intense integrated binding signals in DNA, it was not a stand-out binder in RNA. Furthermore, while probe turn-on reached ~1000× over background in DNA, the range in RNA was about half, suggesting that NCPs in dsRNA are generally less probe-accessible than in dsDNA.

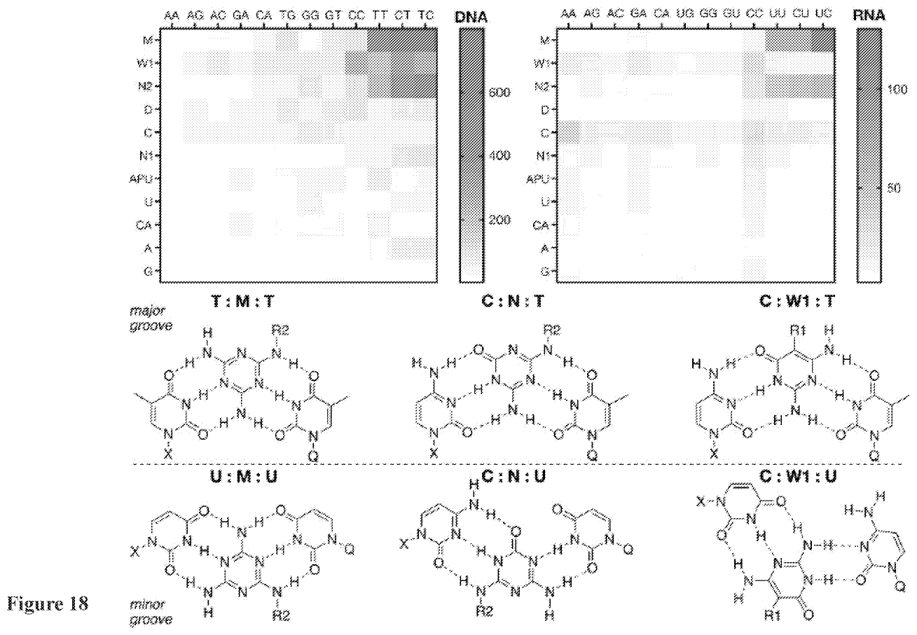

Single NCP sites. The general trends seen in the abasic library played out the single NCP libraries: probe emission with DNA was ~7× more intense than in RNA and triazine probes M-R2 and N—R2 were again the best binders, targeting T/U and C containing sites. Pyrimidine-pyrimidine (YY) NCPs were clearly more accepting of base probes while purine-purine (RR) and pyrimidine-purine (YR/RY) sites were generally unresponsive. Single mismatch stability likely plays a role, with GU the most stable due to wobble pairing and CU/UC/CC mismatches are among the least stable; among YY pairs, UU is the most stabilized by wobble pairing. Probe W1 again showed affinity for CC, TT and TC/CT sites; binding was muted in RNA as seen in the abasic library. The affinity of melamine and ammeline for T/U sites was affirmed, with the affinity for mixed CT and TC sites perhaps indicative of single base pairing rather than base-triple formation. Again, W1 and N1 exhibited distinct behavior, particularly in DNA, where W1 was a strong binder to all YY sites, while N1 did not exhibit clear binding to any site. However, N2 showed good binding to all YY sites except CC. These observations underscore a significant linker effect as well as a fundamental difference between pyrimidine and triazine bases despite similar hydrogen bonding tautomers. In addition, the ~7× fold advantage in probe brightness in DNA over RNA is likely reflective of steric barriers to probe insertion presented by A-form dsRNA, which is more tightly wound than B-form dsDNA. The abasic DNA and RNA libraries are more similar in probe emission intensity, possibly because the abasic site is more inherently accessible than a site with two bases; further, the lack of probe reactivity in purine-abasic, RY and RR sites relative to YY sites is consistent with sterically blocked purine sites. We speculate that probes approach NCPs from the major groove in DNA and from the minor groove in RNA, as these are sterically the most accessible paths in B and A-form helices, respectively. The altered probe binding profiles would thus reflect the distinct hydrogen bonding interfaces offered upon approach from either groove (FIG. 18). One would expect similar melamine binding to TT and UU sites due to the symmetry of both the pyrimidine faces and the triazine itself, while W1 and N would encounter significantly different interactions at the major vs minor grooves. Notably, while W1 binding to TC sites in DNA has been studied, binding to RNA has not been previously reported; in our assay, W1 exhibits no binding to the single UC or CU site in dsRNA (FIG. 18).

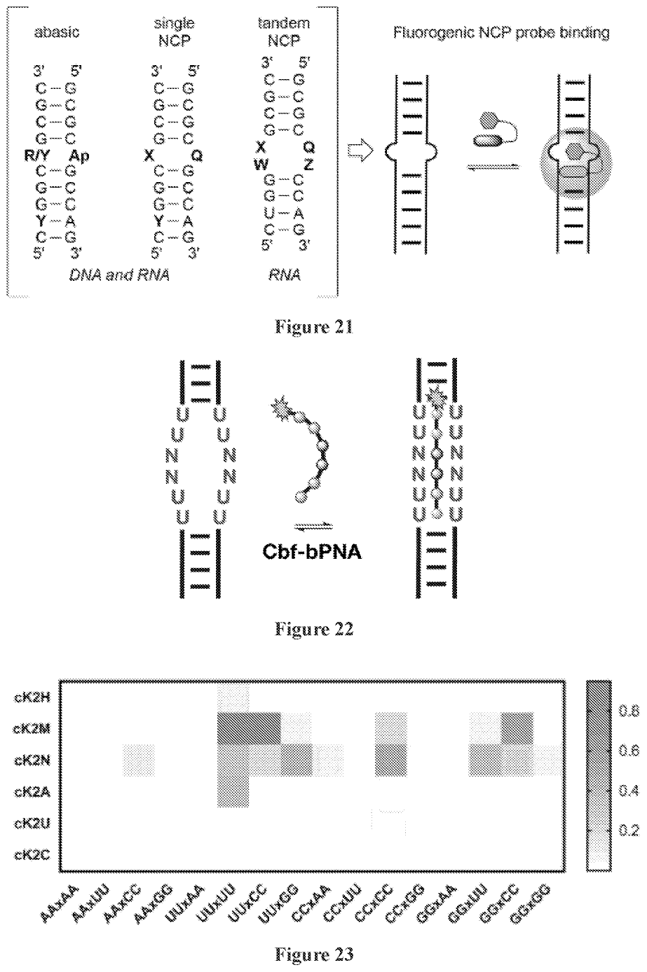

Tandem NCP sites. We focused our subsequent studies on tandem NCP sites in RNA only, as RNA is more likely to contain noncanonical motifs. A library of tandem NCPs in dsRNA, forming 2×2 internal bulges (FIG. 21) was annealed. It was anticipated that 2×2 bulges would be more dynamic than 1×1 bulges and would potentially be more accessible for binding at one or both NCPs, thereby providing a new structural context for probe binding. A limited set of 43 unique 2×2 bulges in dsRNAs was prepared to generally assess the reactivity of RR/RR, RR/RY, RY/RY, RR/YY, YY/YR and YY/YY NCP sites towards our probe library. In contrast to the monotonic output from the single NCP substrates, the tandem NCP library yielded an information-rich set of profiles (FIG. 19) that indicated context-dependent binding properties (FIG. 18). Not only were probes with broad scope binding identified, we found internal bulges that accepted many probes, in particular, AG/CU could be targeted by W1, C, N1, APU, CA and U probes. Notably, C, U, APU and CA did not show any binding in the abasic or single NCP screens, suggesting that tandem NCP motifs are significantly more accessible and/or reactive. We speculate that motifs such as AG/CU are probe-reactive due to the relative instability of the internal bulge such that probe binding provides an energetic benefit. For the specific bulge AG/CU, CU is a particularly destabilizing mismatch while AG is moderately stabilizing; moreover, a size mismatch between RR and YY is destabilizing due to poor stacking overlap. It is reasonable that this relatively unstable motif would present binding opportunities complementary all four native bases, resulting in broad scope of binding among the probes studied. When the tandem NCPs were scored according to size matching of the stacked pairs, we found the strongest probe binding with the largest size mismatch (RR/YY and YY/RR). Generally, probes did not bind well to size matched pairs (eg-RY/RY, YY/YY, RR/RR) (FIG. 18). An exception is N2 binding to UC/CU and UC/CC, which suggests selectivity of N for UC pairs (FIG. 19). However, for reasons not fully understood, N2 did not show binding to UC/CU, which has the lowest stability as calculated with bifold. It appears that fold energetics provide a backdrop to site reactivity, which remains directed by the base recognition element (FIG. 19). For instance, UU/UU did not accept any probes, despite the fact that bPNAs are well-established to bind to UU sites in RNA with M bases; however, M showed a strong fluorescence response with UU/GG. We rationalize this observation by noting that the UU/UU bulge is highly stabilized by wobble pairs and any benefit of melamine binding would be diminished by the loss of existing structure. In contrast, the YY/RR size mismatch in UU/GG renders the motif unstable, leading to a potential energetic benefit upon expansion of the UU site to a UMU triple; this would correct the size mismatch and increase base-stacking against GG. Interestingly, RR pairs have been observed to bridge the structural transition between G-quadruplex or base-triple domains and duplex domains.

While melamine and ammeline were good binders, overall weak binding was observed with the related triazines ammelide and cyanuric acid, possibly due to deprotonation of ammelide and cyanuric acid (pK_{AS}~6.9) while ammeline and melamine are neutral at pH 7. Anionic bases impose a protonation cost to binding and are particularly detrimental to base recognition; however, no changes in binding profiles were observed at pH 4-6.5. Similarly, while W1 (6-aminopseudoisocytidine) exhibited many NCP interactions, APU (6-aminopseudouridine) showed nearly no binding. It is notable that the APU and W1 are the pyrimidine analogs of ammelide and ammeline, respectively, and their reactivity patterns are nearly identical, suggesting that replacement of the exocyclic amine in ammeline and W1 with a keto group in ammelide and APU could be causative in loss of NCP binding, possibly due to introduction of carbonyl lone pair repulsions at the NCP interface.

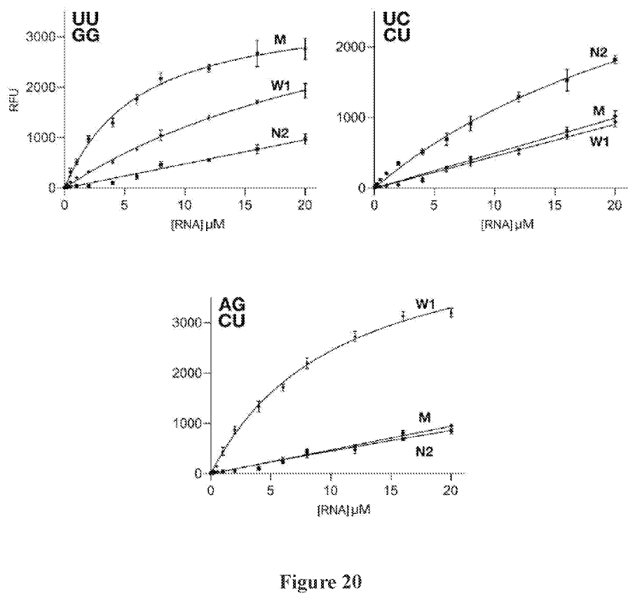

Three tandem NCP sites in dsRNA were selected for careful analysis of affinity: UU/GG, UC/CU and AG/CU. These sites have a clear preferences for M, N2 and W1 probes, respectively (FIG. 19). Using fluorescence readout, binding curves for all three NCP sites and all three probes (M, N2, W1) were generated in triplicate and fit to a 1:1 binding model (FIG. 20). The good fit and low standard deviation supported the 1:1 binding model, and importantly, substantiated the triplicated single concentration measurement data in the screen. As expected, M, N2 and W1 were the best binders for UU/GG, UC/CU and AG/CU, respectively (Table 1). Notably, probes that gave a weak RNA binding signal over free probe background (0-50× turn-on) could not saturate binding.

TABLE 1
Probe affinity to selected tandem NCPs.^{[a]}

| | K_{d} [μM] | | |
|---|---|---|---|
| | UU | AG | UC |
| Probe | GG | CU | CU |
| M | 5.7 (1.3) | >1000 | >1000 |
| N2 | >1000 | >1000 | 33.5 (9.6) |
| W1 | 28.4 (7.2) | 8.9 (2.4) | >1000 |

^{[a]}Standard deviation error from triplicated measurements shown in brackets. Non-saturating binding curves shown as >1000 μM K_{d}. Probe concentration was 500 nM.

### Conclusions

The observations herein indicate that probe binding is influenced by base recognition, NCP stability and accessibility, and underscore the fact that DNA and RNA targeting are entirely distinct problems. We speculate that differences in labeling profiles stem from greater NCP accessibility from the B-form major groove of DNA helices than the A-form minor groove of RNA helices; the probes thus encounter distinct steric challenges and hydrogen bonding interfaces. The abasic and single NCP screens validated expectations regarding hydrogen bond complementarity and also revealed steric limitations of targeting single base pair anomalies with the current probe design. We were both surprised and gratified to observe a broader range of probe reactivity in the tandem NCP library, which we attribute to increased accessibility and energetic driving force for probe binding. Thus, we find a greater diversity of binding selectivities, even among probes that were silent in the abasic and single NCP screens. In each set of screens, M, N and W1 exhibited the strongest binding signals to pyrimidine NCPs (TT, TC, CT, UU, UC, CU). Though apparently overlapping in substrate, the probes demonstrated selectivity in the tandem NCP screen, with M uniquely targeting the UU/GG motif and N uniquely targeting UC/CU and UC/CC motifs, respectively. Non-overlapping targeting is critical for broadening the scope of noncanonical hybridization and the ammeline base appears to be a useful tool to apply in conjunction with melamine. Though silent in the RNA single NCP screen, synthetic pyrimidine W1 has a wide array of substrates in the tandem NCP screen. The RNA targeting profile of W1 has not been previously reported and was found to be surprisingly rich in the tandem NCP screen; W1 could be applied as a “universal base,” capable of binding many different RNA motifs. Likewise, the AG/CU bulge may be a universally accepting motif due to its reactivity with many different probes. Despite the relative promiscuity of the AG/CU internal bulge, it is notable that neither M nor N bind well to this site, suggesting that macromolecular display of a combination M, N and W1 could potentially bind to an internal bulge containing UU, CU and AG NCPs in tandem. The C-probe exhibits a similar profile to W1 (FIG. 19), despite different hydrogen bonding patterns in the neutral base. Interestingly, protonated C(C+) shares the same pattern with W1 so it is possible that this is the active targeting base; however, limited screens under acidic conditions did not enhance or alter binding patterns. Thus, the synthetic and native base probes studied herein have revealed 4 potentially useful recognition elements (M, N, W1, C) for noncanonical hybridization of macromolecular reagents to RNA internal bulges, and studies to incorporate these bases into bPNAs are ongoing. Taken together, these data show that a step-wise, design-based approach can provide insight and complement other strategies for targeting noncanonical RNA sites with synthetic reagents.

### Materials and Methods

General Methods. All chemicals were used without further purification from commercial sources, unless otherwise noted. DNAs and RNAs were purchased from Integrated DNA Technologies (IDT). Nucleic acid strands shorter than 20 nt were used without further purification while longer DNAs/RNAs were purified by TBE-urea denaturing gel. SYBR® gold was purchased from Thermo Fisher Scientific. DNA stock solutions were serially diluted in Milli-Q (18 MΩ) water and concentrations were determined by solution absorbance (260 nm) with a Thermo Fisher Nanodrop 2000. Sample fluorescence was measured on Thermo Fisher Nanodrop 3300. Buffer for fluorescence turn-on (1×) contained: HEPES (pH=7.5) 50 mM, NaCl 100 mM. Analytical and semi-preparative HPLC was carried out with Cis reverse phase columns with the following HPLC solvents: solvent A (99% Milli-Q H₂O, 1% HPLC grade acetonitrile, 0.1% trifluoroacetic acid) and solvent B (10% Milli-Q H₂O, 90% HPLC grade acetonitrile, 0.07% trifluoroacetic acid).

### Nucleic Acid Sequences

DNA single mismatches. 5′ and 3′ strands were annealed together into duplexes before the fluorescence turn-on experiment. Mismatch site X-Q is bolded and underlined.

5′ C (left strand):
(SEQ ID NO. 13)
5′-CTGGC C GCGC-3′
5′ G (left strand):
(SEQ ID NO. 14)
5′-CTGGC G GCGC-3′
5′ A (left strand):
(SEQ ID NO. 15)
5′-CTGGC A GCGC-3′
5′ T (left strand):
(SEQ ID NO. 16)
5′-CTGGC T GCGC-3′
3′ C (right strand):
(SEQ ID NO. 17)
5′-GCGC C GCCAG-3′
3′ G (right strand):
(SEQ ID NO. 18)
5′-GCGC G GCCAG-3′
3′ A (right strand):
(SEQ ID NO. 19)
5′-GCGC A GCCAG-3′
3′ T (right strand):
(SEQ ID NO. 20)
5′-GCGC T GCCAG-3′

RNA single mismatches, S′ and 3′ strands were annealed together into duplexes before the fluorescence turn-on experiment. Mismatch site X-Q is bolded and underlined.

5′ C (left strand):
(SEQ ID NO. 21)
5′-CUGGC C GCGC-3′
5′ G (left strand):
(SEQ ID NO. 22)
5′-CUGGC G GCGC-3′
5′ A (left strand):
(SEQ ID NO. 23)
5′-CUGGC A GCGC-3′
5′ U (left strand):
(SEQ ID NO. 24)
5′-CUGGC U GCGC-3′
3′ C (right strand):
(SEQ ID NO. 25)
5′-GCGC C GCCAG-3′
3′ G (right strand):
(SEQ ID NO. 26)
5′-GCGC G GCCAG-3′
3′ A (right strand):
(SEQ ID NO. 27)
5′-GCGC A GCCAG-3′
3′ U (right strand):
(SEQ ID NO. 28)
5′-GCGC U GCCAG-3′

Abasic (apurine/apyrimidine) site. DNA and RNA abasic strands of the sequence indicated below were annealed with each DNA or RNA S′ strand before the fluorescence turn-on experiment. The AP site is indicated below.

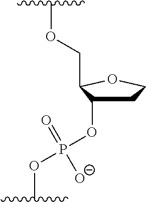

3′ AP (right strand):
(SEQ ID NO. 29)
5′-GCGC [AP] GCCAG-3′

Tandem RNA strands. S′ and 3′ strands were annealed together into duplexes before the fluorescence turn-on experiment. Mismatch sites X-Q and W—Z are bolded and underlined.

5′ GU (left strand):
(SEQ ID NO. 30)
5′-CUGG GU GCGC-3′
5′ UU (left strand):
(SEQ ID NO. 31)
5′-CUGG UU GCGC-3′
5′ CA (left strand):
(SEQ ID NO. 32)
5′-CUGG CA GCGC-3′
5′ CU (left strand):
(SEQ ID NO. 33)
5′-CUGG CU GCGC-3′
5′ AA (left strand):
(SEQ ID NO. 34)
5′-CUGG AA GCGC-3′
5′ UA (left strand):
(SEQ ID NO. 35)
5′-CUGG UA GCGC-3′
5′ CC (left strand):
(SEQ ID NO. 36)
5′-CUGG CC GCGC-3′
3′ GU (right strand):
(SEQ ID NO. 37)
5′-GCGC GU CCAG-3′
3′ UG (right strand):
(SEQ ID NO. 38)
5′-GCGC UG CCAG-3′
3′ UU (right strand):
(SEQ ID NO. 39)
5′-GCGC UU CCAG-3′
3′ GA (right strand):
(SEQ ID NO. 40)
5′-GCGC GA CCAG-3′
3′ CU (right strand):
(SEQ ID NO. 41)
5′-GCGC CU CCAG-3′
3′ GC(right strand):
(SEQ ID NO. 42)
5′-GCGC GC CCAG-3′
3′ CA (right strand):
(SEQ ID NO. 43)
5′-GCGC CA CCAG-3′
3′ UA (right strand):
(SEQ ID NO. 44)
5′-GCGC UA CCAG-3′
3′ AC(right strand):
(SEQ ID NO. 45)
5′-GCGC AC CCAG-3′
3 ′ CC (right strand):
(SEQ ID NO. 46)
5′-GCGC CC CCAG-3′
3′ AA (right strand):
(SEQ ID NO. 47)
5′-GCGC AA CCAG-3′

### Synthetic Procedures

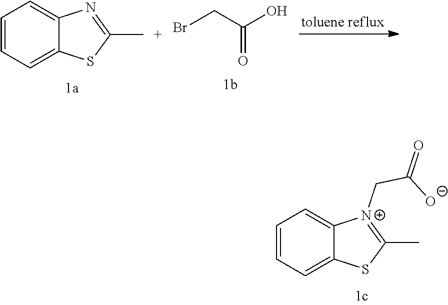

Synthesis of 2-(2-Methylbenzo[d]thiazol-3-ium-3-yl)acetate (1c). A mixture of 2-methylbenzothiazole (1a, 4.78 g, 32 mmol, 1.0 eq.) and bromoacetic acid (1b, 6.5 g, 47 mmol, 1.5 eq.) in toluene (100 ml) was heated at reflux overnight. After cooling to room temperature, the resulting mixture was filtered and the solid was washed with toluene (3×5 ml) to afford the product (1c, 6.5 g, 98%) after drying.

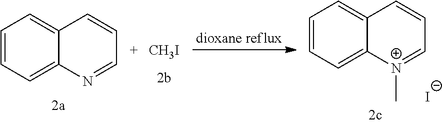

Synthesis of 1-methylquinolin-1-ium iodide (2c). A mixture of quinoline (2a, 2 g, 15.5 mmol, 1.0 eq.) and iodomethane (2b, 2 ml, 32.2 mmol, 2.1 eq.) in 1,4-dioxane (150 ml) was heated at reflux for 1 hr. After cooling to room temperature, the resulting mixture was filtered and the solid was washed with diethyl ether (3×5 ml) and hexanes (3×5 ml), affording the product (2c, 4 g, 95%) after drying.

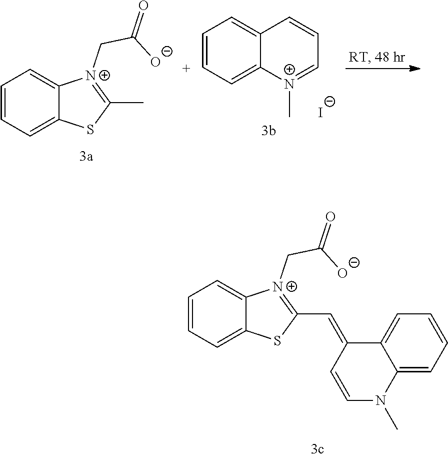 [SMILES: Cc1sc2ccccc2[n+]1CC(=O)[O-] ; C[n+]1cccc2c1=C[C+]=CC=2 ; [I-] ; CN1C=C/C(=C\c2sc3ccccc3[n+]2CC(=O)[O-])c2ccccc21]

Synthesis of TO-acetate (3c). 2-(2-Methylbenzo[d]thiazol-3-ium-3-yl)acetate (3a, 2.14 g, 10.4 mmol, 1.4 eq.), 1-methylquinolin-1-ium iodide (3b, 2 g, 7.4 mmol, 1 eq.) and triethylamine (2.24 g, 22.2 mmol, 3 eq.) were added in a round bottom flask with 30 ml dichloromethane. The mixture was stirred at room temperature for 48 hr. After 48 hr, the mixture was concentrated and 16 ml ethanol and 4 ml diethyl ether were added. The resulting mixture was stirred at 4° C. overnight. The resulting precipitate was collected by filtration and washed with diethyl ether (3×8 ml). The solid was then dissolved in 12 ml methanol with 3 ml water and was stirred overnight at 4° C. The resulting precipitate was collected by filtration and washed with water (3×8 ml). Then the solid was resuspended in acetone and stirred at room temperature for 1 hr. The final product was obtained by filtration and was washed with acetone (3×8 ml) and dried in vacuum to afford TO-acetate as red solid (3c, 160 mg, 6.4%). ¹H NMR (DMSO-d₆): 8.54 (1H, d); 8.48 (1H, d); 7.98 (1H, d); 7.94 (1H, d); 7.86 (1H, t); 7.64 (1H, d); 7.55 (2H, m); 7.38 (1H, t); 7.18 (1H, d); 6.77 (1H, s); 5.09 (2H, s); 4.10 (3H, s). HRMS (ESI): Mass calculated: [M+H⁺]=349.1005; Mass found: [M+H]⁺=349.1004.

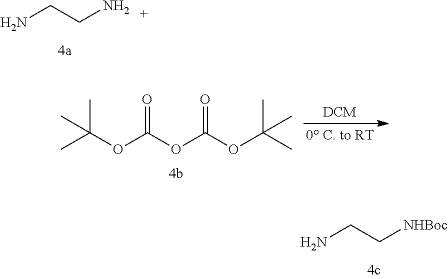

Synthesis of Boc-ethylenediamine (4c). To a stirring dichloromethane (200 ml) solution containing ethylenediamine (4a, 20 g, 0.33 mol, 5 eq.) was added di-tert-butyl dicarbonate (4b, 14.5 g, 66.6 mmol, 1 eq.) dichloromethane solution (20 ml) dropwise at 0° C. in 2 hr. After addition, the solution was warmed up to room temperature and stirred overnight. Then the reaction was concentrated in vacuum and dissolved in saturated sodium bicarbonate solution. The resulting mixture was extracted with dichloromethane (3×30 ml) and the combined organic phase was extracted with brine, dried with sodium sulfate and evaporated to dryness in vacuum, yielding the product (4c, 10.2 g, 96%) as colorless liquid. ¹H NMR (DMSO-d₆): 6.73 (1H, s), 2.90 (2H, q); 2.52 (2H, t); 1.40 (2H, t); 1.38 (9H, s).

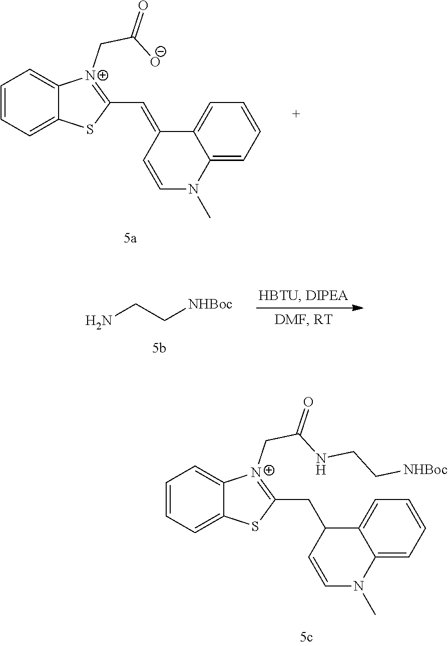 [SMILES: CN1C=C/C(=C\c2sc3ccccc3[n+]2CC(=O)[O-])c2ccccc21 ; CC(C)(C)OC(=O)NCCN ; CN1C=CC(Cc2sc3ccccc3[n+]2CC(=O)NCCNC(=O)OC(C)(C)C)c2ccccc21]

Synthesis of TO-ethylenediamine-Boc (5c). TO-acetate (5a, 100 mg, 0.29 mmol, 1 eq.), N,N-Diisopropylethylamine (74 mg, 0.58 mmol, 2 eq.) and HBTU (121 mg, 0.32 mmol, 1.1 eq) were added to DMF (5 ml) and stirred for 5 min. Boc-ethylenediamine (5b, 70 mg, 0.43 mmol, 1.5 eq.) was added to the solution and stirred overnight in darkness. The mixture was concentrated in vacuum and the crude product was dissolved in DCM and loaded to silica gel column for purification, affording orange solid (5c, 115 mg, 81%) as pure product. ¹H NMR (DMSO-d₆): 8.54 (1H, d); 8.48 (1H, d); 7.98 (1H, d); 7.94 (1H, d); 7.85 (1H, t); 7.62 (1H, d); 7.57 (2H, m); 7.37 (1H, t); 7.16 (1H, d); 6.75 (1H, s); 6.30 (1H, s); 5.97 (1H, s); 5.27 (2H, s); 4.10 (3H, s); 3.22 (2H, t); 2.96 (2H, t); 1.31 (9H, s). HRMS (ESI): Mass calculated: [M+H⁺]=491.2111; Mass found: [M+H⁺]=491.2108

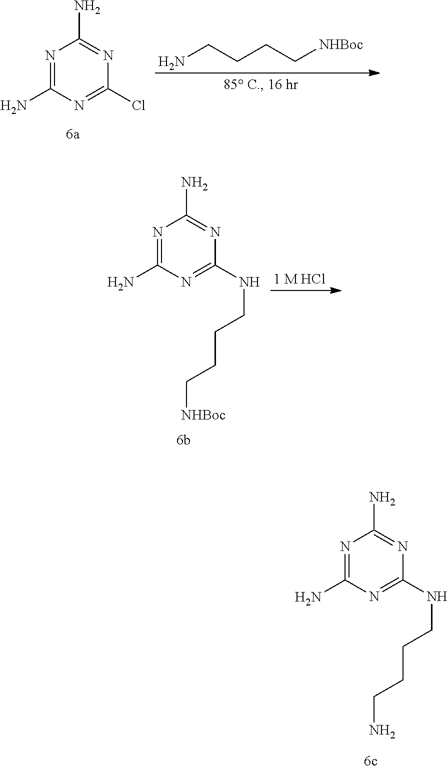

Synthesis of N²-(4-aminobutyl)-1,3,5-triazine-2,4,6-triamine (6c). 6-chloro-1,3,5-triazine-2,4-diamine (6a, 1.45 g, 10 mmol, 1 eq.) was added to a aqueous solution containing tert-butyl (4-aminobutyl) carbamate (2.25 g, 12 mmol, 1.2 eq.) and sodium bicarbonate (1.85 g, 22 mmol, 2.2 eq.). The mixture was stirred at 85° C. for 16 hr. After cooling to room temperature, the product (6b) was filtered, washed with cold water and dried. The resulting solid was dissolved in 1 M HCl and stirred for 2 hr. The reaction was dried in vacuum to afford white solid as pure product (6c, 1.76 g, 89%). ¹H NMR (D₂O): 1.66 (4H, m); 3.36 (2H, t); 3.68 (2H, t). HRMS (ESI): Mass calculated: [M+H⁺]=198.1462; Mass found: [M+H⁺]=198.1476

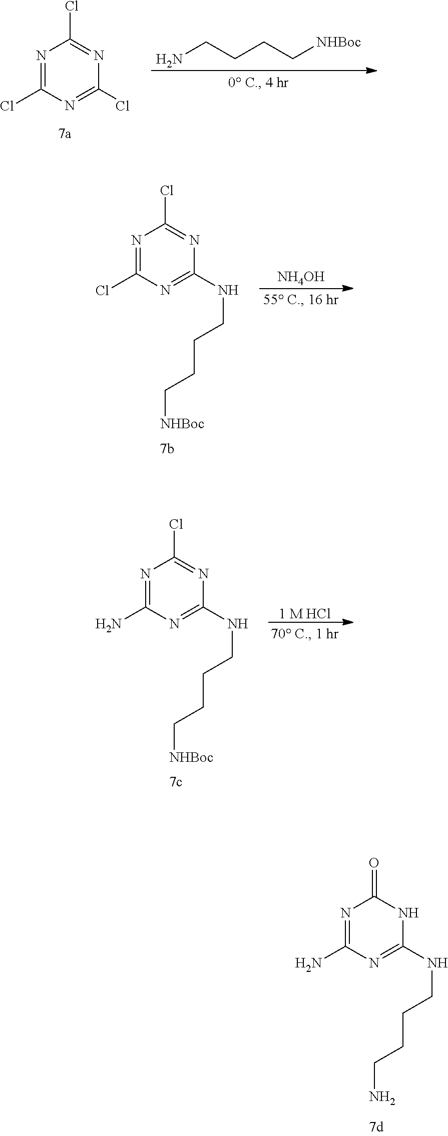

Synthesis of 4-amino-6-((4-aminobutyl)amino)-1,3,5-triazin-2 (1H)-one (7d). Triazine Chloride (7a, 2.0 g, 10.98 mmol, 1 eq.) was dissolved in acetone (30 ml) and iced cold water (40 ml) and the reaction mixture was cooled to 0° C. A solution of tert-butyl (4-aminobutyl) carbamate (2.0 g, 15 mmol, 1.5 eq.) was added at the same temperature. The resulting mixture was stirred at 0° C. for 4 h. The white solid was filtered, washed with water (3×20 ml) and dried under vacuum to afford a pure intermediate 7b that was used for the following reaction. 7b (1.0 g, 2.98 mmol) was dissolved in acetone (20 ml) and iced cold water (20 ml) at 0° C. 28% NH₄OH solution was added dropwise to the reaction mixture. The resulting mixture was stirred at the same temperature for 10 min and warmed to 55° C. for 16 hr. The reaction mixture was cooled to 0° C. for 30 mins and the solid was collected by vacuum filtration. The product was washed with water (3×20 ml) and dried under vacuum to afford pure product 7c. 7e (0.5 g, 1.4 mmol) was dissolved in 1 M HCl (20 ml) and heated to 70° C. for 1 hr. The reaction was cooled to room temperature and the water was removed by using Na stream until 5 ml solvent was left. The resulting mixture was freeze-dried to afford the product 7d (0.28 g, 96%) as white solid. ¹H NMR (DMSO-d₆): 8.81 (1H, t); 8.14 (2H, br); 3.89 (2H, q); 2.78 (2H, t); 1.57 (4H, m).

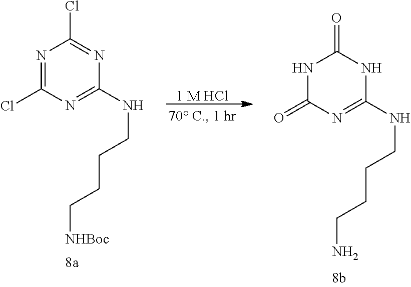

Synthesis of 6-((4-aminobutyl)amino)-1,3,5-triazine-2,4 (1H_{1,3}H)-dione (8b). 8a (0.5 g, 1.4 mmol) was dissolved in 1 M HCl (20 mL) and heated to 70° C. for 1 hr. The reaction was cooled to room temperature and removed the water by using N2 stream until 5 ml solvent was left. The resulting mixture was freeze dried to afford the product 8b (0.27 g, 93%) as white solid. ¹H NMR (DMSO-d₆): 8.12 (3H, m); 3.41 (2H, t); 2.80 (2H, t); 1.57 (4H, m).

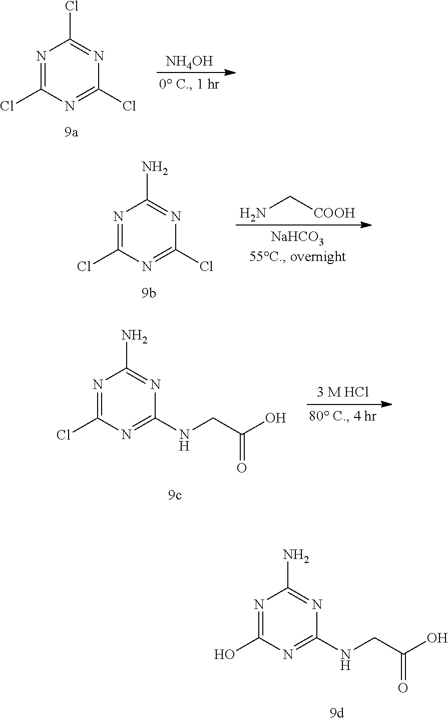 [SMILES: Clc1nc(Cl)nc(Cl)n1 ; "NH4OH" ; Nc1nc(Cl)nc(Cl)n1 ; NCC(=O)O ; Nc1nc(Cl)nc(NCC(=O)O)n1 ; Nc1nc(O)nc(NCC(=O)O)n1]

Synthesis of (4-amino-6-hydroxy-1,3,5-triazin-2-yl)glycine (9d). Cyanuric chloride (9a, 15 g, 82 mmol) was dissolved in acetone (~50 ml, until fully dissolved) and poured into cold water (50 ml) with stirring in ice bath to form a white suspension. Aqueous ammonia hydroxide (30%, 18 ml) was added dropwise to the suspension in the ice bath with stirring. The reaction was stirred at 0° C. for 1 hr and the precipitate was filtered, washed with ice-cold water and dried in vacuum. The resulting white solid (4,6-dichloro-1,3,5-triazin-2-amine, 9b) (11 g, 81% yield, 65 mmol, 1 eq.) was added to an aqueous solution (100 ml) containing glycine (5.3 g, 71.5 mmol, 1.1 eq.) and sodium bicarbonate (13.6 g, 162.5 mmol, 2.5 eq.) and the mixture was stirred at 55° C. overnight. The mixture was concentrated to ~50 ml and the pH was adjusted to 3 with 6 M HCl. The precipitate was collected by filtration, washed with acidified water (3×5 ml) and dried in vacuum to afford (4-amino-6-chloro-1,3,5-triazin-2-yl)glycine as white solid (9c, 9.1 g, 45 mmol, 69%). Then the solid was added to 3 M HCl (50 ml) and was stirred at 80° C. for 4 hr. The solvent was evaporated to yield the title product as white solid (9d, 8.3 g, 99%). ¹H NMR (DMSO-d₆): 10.26 (1H, s); 6.94 (2H, br); 6.57 (1H, t); 3.81 (2H, d). HRMS (ESI): Mass calculated: [M⁻]=184.0476; Mass found: [M⁻]=184.0473

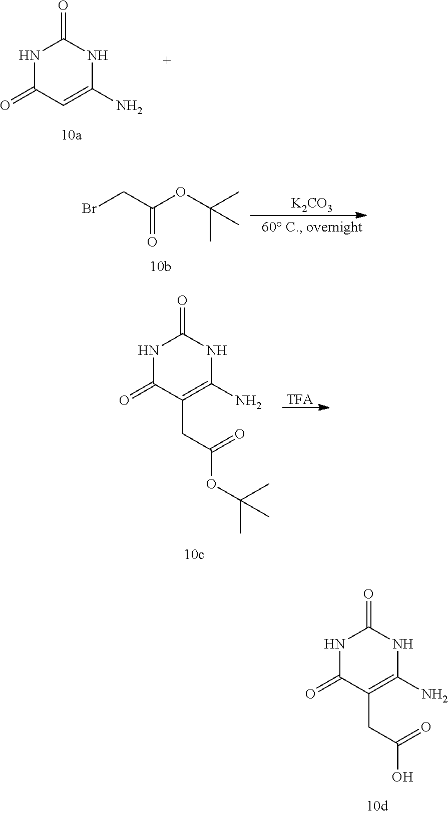

Synthesis of tert-butyl 2-(6-amino-2,4-dioxo-1,2,3,4-tetrahydropyrimidin-5-yl)acetate (10c). 6-aminopyrimidine-2,4 (1H,3H)-dione (10a, 1.27 g, 10 mmol, 1 eq.), potassium carbonate (2.76 g, 20 mmol, 2 eq.) and tert-butyl 2-bromoacetate (10b, 2.93 g, 15 mmol, 1.5 eq.) were added to DMF (40 ml) and stirred at 60° C. overnight. The solvent was removed and the residue was resuspended in water and the crude product was collected by filtration and dried in vacuum. The crude product was then loaded to silica gel column for purification, affording purified product (10c, 0.89 g, 37%) as white solid after drying. The product was characterized by ¹H NMR, ¹³C NMR, C—H HSQC, ¹³C APT, ¹³C APT-DEPT90 and HRMS (ESI). ¹H NMR (DMSO-d₆): 10.23 (1H, s); 9.98 (1H, s); 6.04 (2H, s); 3.08 (2H, $); 1.38 (9H, s). ¹³C NMR (DMSO-d₆): 170.57; 164.01; 152.28; 150.16; 79.75; 79.29; 28.95; 27.84. HRMS (ESI): Mass calculated: [M+H]⁺=242.1135, [M+Na⁺]=264.0955; Mass found: [M+H]⁺=242.1137, [M+Na⁺]=264.0955. 0.1 g 10c was added to 2 ml TFA and stirred for 2 hr. Then TFA was removed by N₂ stream and the residue (10d) was characterized by ESI and used for the following coupling reaction. HRMS (ESI): Mass calculated: [M+H]⁺=186.0509; Mass found: [M+H⁺]=186.0511.

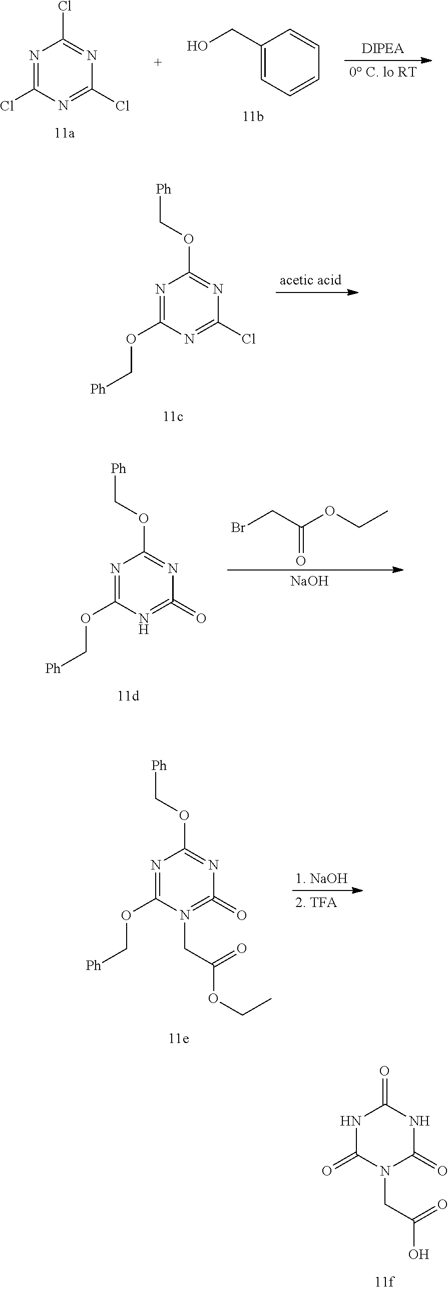

Synthesis of Cyanuric acid-acetate. Benzyl alcohol (11b, 38.3 ml, 0.36 M, 4 eq.) and N,N-Diisopropylethylamine (64.2 ml, 0.36 M, 4 eq.) were added to chloroform (34 ml) and the resulting mixture was dropwisely added to the chloroform solution (68 ml) containing cyanuric chloride (11a, 17 g, 0.09 M, 1 eq.) at 0° C. The reaction was warmed to room temperature and stirred overnight. 200 ml saturated NH₄Cl solution was added to the reaction and the organic layer was collected, washed with brine (2×100 ml) and dried in vacuum. The crude product 11c was used without further purification. The crude product (11c) was then added to a methanol solution (385 ml) containing n-methylmorpholine (40.2 ml, 0.4 M, 4 eq.) and acetic acid (10.6 ml, 0.18 M, 1.8 eq.) at 0° C. The reaction was then warmed up to room temperature and stirred for 1 hr. Acetic acid (10.6 ml, 0.18 M, 1.8 eq.) was added to the reaction and the solvent was removed in vacuum. 150 ml chloroform and 150 ml 1 M HCl was added to the residue and the organic layer was separated, washed with saturated saline solution and dried under reduced pressure, obtaining 4,6-bis(benzyloxy)-1,3,5-triazin-2 (1H)-one (11d) as crude product. The crude 4,6-bis(benzyloxy)-1,3,5-triazin-2(1H)-one (11d, 0.5 g) and NaOH (0.78 g) was added to 4.5 ml THF at 0° C. Then ethyl bromoacetate (0.36 ml) was added to the mixture and stirred overnight at room temperature. THF was removed and saturated NH₄Cl and ethyl acetate were added to the residue. The organic layer was separated, dried and concentrated in vacuum. The resulting crude product (Ile) was purified by silica gel column, affording ethyl 2-(4,6-bis(benzyloxy)-2-oxo-1,3,5-triazin-1 (2H)-yl)acetate (Ile, 0.25 g, 40%) as pure product. ¹H NMR (CDCl₃): 7.47 (2H, m); 7.37 (8H, m); 5.46 (4H, s); 4.68 (2H, s); 4.15 (2H, q); 1.20 (3H, t). HRMS (ESI): Mass calculated: [M+H]⁺=396.1554, [2 M+H⁺]=791.3035; Mass found: [M+H⁺]=396.1527, [2 M+H⁺]=791.2964.

Then Ile was added to 2 M NaOH (2 ml) and stirred for 4 hr. Then the solution pH was adjusted to pH 4 with concentrated HCl and the resulting precipitate was filtered and dried in vacuum. The resulting solid was added to TFA (2 ml) and stirred for 8 hr, dried with nitrogen stream and the residue (11f) was used for the following coupling reaction. HRMS (ESI): Mass calculated: [M⁻]=186.0156; Mass found: [M⁻]=186.0153.

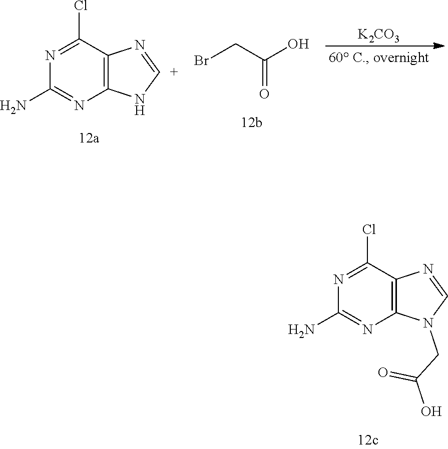

Synthesis of 2-(2-amino-6-chloro-9H-purin-9-yl) acetic acid (12c). 6-chloro-9H-purin-2-amine (12a, 5 g, 29.5 mmol, 1 eq.) and potassium carbonate (12.8 g, 93 mmol, 3.1 eq.) were added to 100 ml DMF with stirring. Bromoacetic acid (12b, 4.6 g, 33.6 mmol, 1.1 eq.) dissolved in 5 ml DMF was added dropwise into the previous DMF suspension. After addition, the resulting mixture was heated to 60° C. overnight. Then 150 ml water was added to the resulting suspension and stirred for 10 min. The mixture was then filtered and the filtrate was collected and acidified to pH~3 with 6 M HCl in an ice bath. The precipitate was filtered, washed with pH 3 water (3×5 ml) and dried in vacuum to give the product (12c, 4.2 g, 62%) as white solid. ¹H NMR (DMSO-d₆): 13.19 (1H, s); 8.10 (1H, s); 6.92 (2H, s); 4.81 (2H, s). HRMS (ESI): Mass calculated: [M⁻]=226.0137; Mass found: [M⁻]=226.0131.

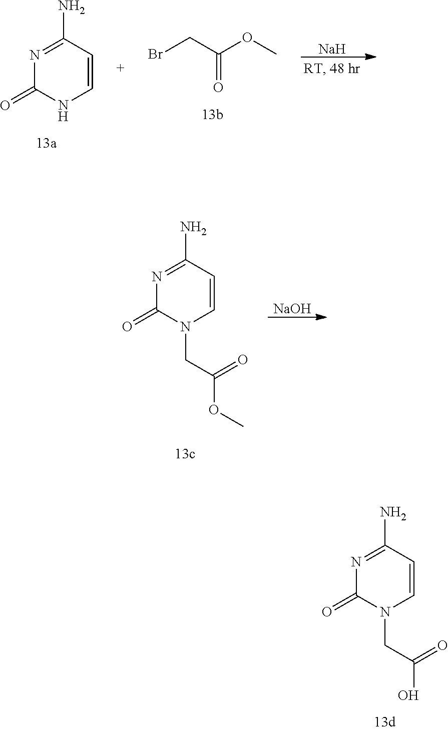

Synthesis of C-acetate (13d). To a suspension of cytosine (13a, 5 g, 45 mmol, 1 eq.) in DMF (100 ml) was added NaH (1.08 g, 45 mmol, 1 eq.) under N₂ at 0° C. The mixture was stirred at room temperature for 2 hr and methyl bromoacetate (13b, 7.6 g, 50 mmol, 1.1 eq) was added. The mixture was stirred at room temperature for 48 hr. Then the solvent was removed in vacuum and the residue was triturated in water (100 ml) and filtered to afford crude C-acetate methyl ester 13c. The crude C-acetate methyl ester was directly added into 30 ml 2 M NaOH solution and stirred for 4 hr. Then the solution pH was adjusted to pH 4 with concentrated HCl and the resulting precipitate was filtered, washed with acidified water and dried in vacuum to afford C-acetate (13d, 4.5 g, 59%) as white solid. ¹H NMR (DMSO-d₆): 7.55 (1H, d); 7.14 (2H, br); 5.67 (1H, d); 4.35 (2H, s). HRMS (ESI): [M+H⁺]=170.0560, [M+Na⁺]=192.0380; Mass found: [M+H⁺]=170.0561, [M+Na⁺]=192.0384.

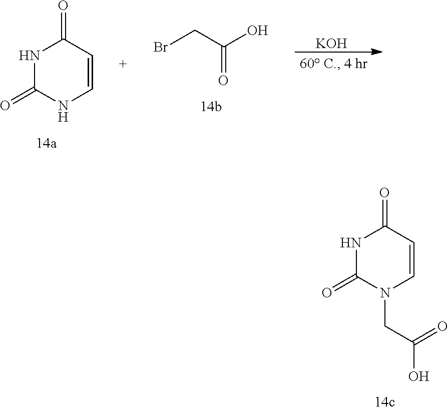 [SMILES: O=c1cc[nH]c(=O)[nH]1 ; O=C(O)CBr ; "KOH" ; O=C(O)Cn1ccc(=O)[nH]c1=O]

Synthesis of U-acetate (14c). Bromoacetic acid (14b, 8.3 g, 60 mmol, 1.2 eq.) in water (15 ml) was added dropwise into an aqueous solution (30 ml) containing uracil (14a, 5.6 g, 50 mmol, 1 eq.) and potassium hydroxide (2.8 g, 100 mmol, 2 eq.) at 60° C. The mixture was stirred at 60° C. for 4 hr and cooled to room temperature. The solution was adjusted to pH 2 with 6 M HCl and the resulting precipitate was filtered and washed with water (3×3 ml) and diethyl ether (5 ml). The solid was dried in vacuum to afford the product (14c, 6.4 g, 75%) as white solid. ¹H NMR (DMSO-d₆): 13.13 (1H, s); 11.33 (1H, s); 7.62 (1H, d); 5.60 (1H, d); 4.41 (2H, s). HRMS (ESI): Mass calculated: [M+H⁺]=171.0400, [M+Na⁺]=193.0220; Mass found: [M+H⁺]=171.0402, [M+Na⁺]=193.0221.

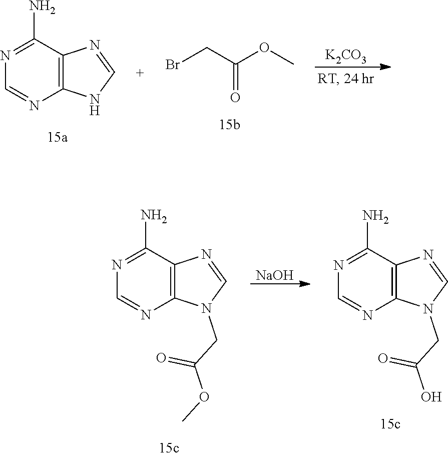

Synthesis of A-acetate (15d). Adenine (15a, 3 g, 22.2 mmol, 1 eq.) and potassium carbonate (1 g, 44.4 mmol, 2 eq.) were added to DMF (100 ml) and stirred for 1 hr. Methyl bromoacetate (15b, 5 g, 33.3 mmol, 1.5 eq.) in DMF (5 ml) was added to the mixture dropwise. The reaction was stirred at room temperature for 24 hr. Then the solvent was removed in vacuum and the residue was triturated in water (100 ml) and filtered to afford crude A-acetate methyl ester 15c. The crude A-acetate methyl ester was directly added into 30 ml 2 M NaOH solution and stirred for 4 hr. Then the solution pH was adjusted to pH 3 with concentrated HCl and the resulting precipitate was filtered, washed with acidified water and dried in vacuum to afford A-acetate (15d, 2.9 g, 69%) as off-white solid. 1H NMR (DMSO-d₆): 8.13 (1H, s); 8.10 (1H, s); 7.22 (2H, s); 4.95 (2H, s). HRMS (ESI): Mass calculated: [M+H⁺]=194.0673, Mass found: [M+H]⁺=194.0666.

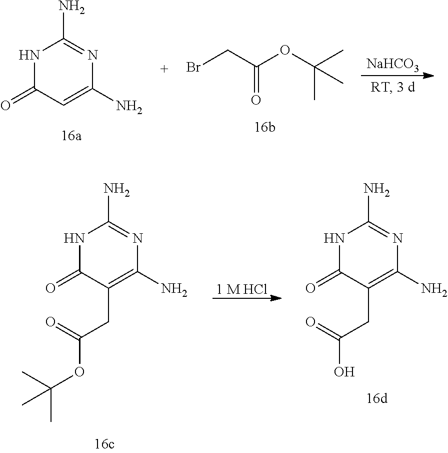

Synthesis of W1-acetate (16d). 2,6-diaminopyrimidin-4 (3H)-one (16a, 2.52 g, 20 mmol, 1 eq.), sodium bicarbonate (2 g, 24 mmol, 1.2 eq.) and tert butyl bromoacetate (16b, 3.9 g, 20 mmol, 1 wq.) were added to DMF (50 ml). The reaction was stirred for 3 days at room temperature. Then the solvent was removed under reduced pressure and the residue was poured into 160 ml of hot water. The product 16c (3.3 g, 70%) was crystallized after cooling as light yellow solid. The solid was then added to 20 ml 1 M HCl and stirred for 2 hr. Then the solvent was dried in lyophilizer, yielding W1-acetate (16d) as yellow solid. ¹H NMR (DMSO-d₆): 9.97 (1H, s); 6.10 (2H, s); 5.75 (2H, s); 3.12 (2H, s). HRMS (ESI): Mass calculated: [M+H⁺]=185.0669; Mass found: [M+H]⁺=185.0667.

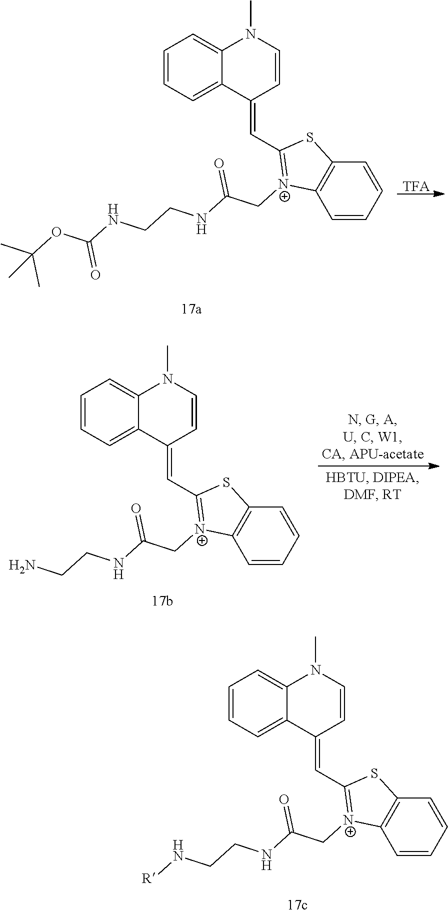

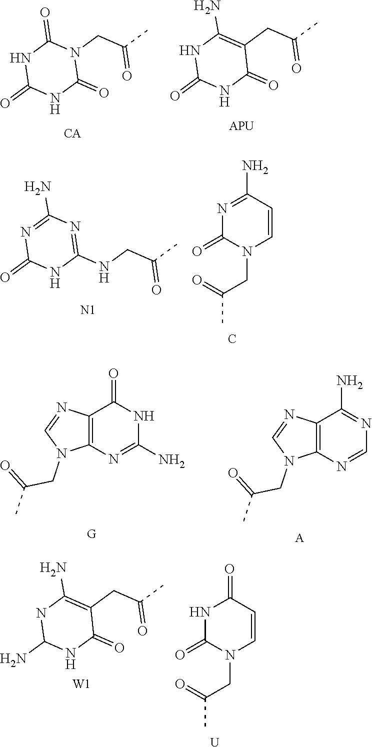

General procedure for R1 probe synthesis. The procedures were similar for R1 derivative synthesis. C was used as an example. TO-ethylenediamine-Boc (17a, 9.8 mg, 0.02 mmol, 1 eq.) was added to trifluoroacetic acid (1 ml) and the reaction was stirred for 4 hr at room temperature. Then TFA was removed by N2 stream and the residue 17b was dissolved in DMF (1.5 ml). N,N-Diisopropylethylamine (20 mg, 0.2 mmol, 10 eq.) and C-acetate (5.1 mg, 0.03 mmol, 1.5 eq.) were added to DMF and stirred for 2 min. HBTU (12 mg, 0.03 mmol, 1.5 eq.) was added to the mixture and the reaction was stirred overnight. The solvent was removed by N2 stream and the residue was resuspended in HPLC solvent A (90% acetonitrile, 10% water, 0.1% TFA), centrifuged and the supernatant was purified by HPLC. The solubility of the R1 probes were low in HPLC solvent during the purification step (estimated solubility <0.2 mg/ml), resulting in a low quantity of purified products. Therefore, HPLC-ESI was used for characterization of the TO conjugated probes.

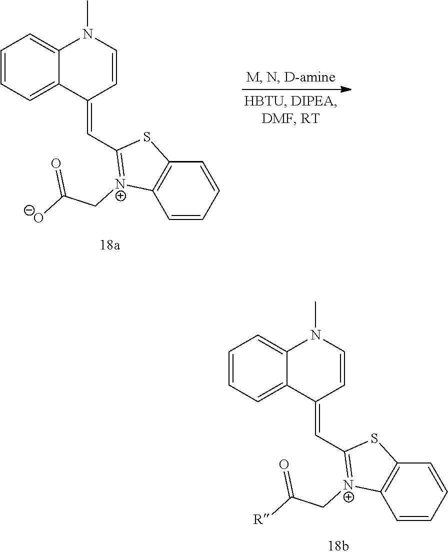

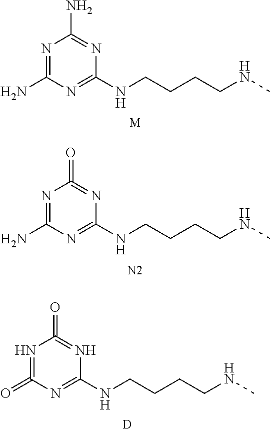

General procedure for making R2 probes. The procedures were similar for R2 derivative synthesis. M was used as an example. TO-acetate (18a, 7 mg, 0.02 mmol, 1 eq.), N2-(4-aminobutyl)-1,3,5-triazine-2,4,6-triamine (5.9 mg, 0.03 mmol, 1.5 eq.) and N,N-Diisopropylethylamine (10 mg, 0.1 mmol, 5 eq.) were added to DMF (1.5 ml) with stirring. HBTU (12 mg, 0.03 mmol, 1.5 eq.) was added to the solution and the reaction was stirred overnight at room temperature. Then DMF was removed by N2 stream and the residue was resuspended in HPLC solvent A (90% acetonitrile, 10% water, 0.1% TFA), centrifuged and the supernatant was purified by HPLC. HPLC-ESI was used for characterization of the TO conjugated probes.

Fluorescence Measurements. Samples for screening were prepared as solutions with 50 mM HEPES (pH 7.5), 100 mM NaCl, 2 μM dsDNA/dsRNA, 2 μM probe (4 μM probe with tandem mismatch). Prior to use, nucleic acids were annealed (95° C./5 min, slow cool to RT over 2 h) to pre-form the duplex. Prepared samples were incubated at room temperature for 30 min before measuring fluorescence (Thermo Fisher Nanodrop 3300, excitation/emission=470/522 nm). For each measurement, 2 μl of sample was applied to the center of the nanodrop probe. All fluorescence values are the average of triplicate measurements, starting from fresh sample preparation. Error bars indicate standard deviation. The relative intensity (turn-on) was calculated using the equation below.

$$relative F = \frac{RFU of probe with DNA / RNA}{RFU of probe only}$$

Binding Affinity. Probes (M, N2, W1) were held at a constant concentration of 500 nM and treated with RNA to final concentrations from 0 to 20 μM in buffer (50 mM. HEPES, pH 7.5, 100 mM NaCl). The experiments were triplicated from sample preparation and error bars indicate standard deviation. The data were fit using the equation below to obtain dissociation constant K_{d}.

$$RFU = \frac{m * ( Kd + x + 500 ) - m * sqrt [ sqr ( Kd + x + 500 ) - 4 * x * 500 ]}{2}$$

### REFERENCES

[1] Miao S, Liang Y, Rundell S, Bhunia D, Devari S, Munyaradzi O, Bong D, Biopolymers 2021, 112, e23399.
[2] Lemieux S, Major F, Nucleic Acids Res. 2002, 30, 4250-4263.
[3] Kumpina I, Brodyagin N, Mackay J A, Kennedy S D, Katkevics M, Rozners E, J. Org. Chem. 2019, 84, 13276-13298.
[4] Zengeya T, Gupta P, Rozners E, Angew. Chem. Int. Ed. 2012, 51, 12593-12596.
[5] Urbach A R, Dervan P B, Proceedings of the National Academy of Sciences 2001, 98, 4343-4348.
[6] Turner J M, Swalley S E, Baird E E, Dervan P B, J. Am. Chem. Soc. 1998, 120, 6219-6226.
[7] Dervan P B, Edelson B S, Curr. Opin. Struct. Biol. 2003, 13, 284-299.
[8] Trauger J W, Baird E E, Dervan P B, Nature 1996, 382, 559-561.
[9] Benner S A, Karalkar N B, Hoshika S, Laos R, Shaw R W, Matsuura M, Fajardo D, Moussatche P, Cold Spring Harb. Perspect. Biol. 2016, 8, DOI 10.1101/cshperspect.a023770.
[10] Benner S A, Battersby T R, Eschgfaller B, Hutter D, Kodra J T, Lutz S, Arslan T, Baschlin D K, Blattler M, Egli M, Hammer C, Held H A, Horlacher J, Huang Z, Hyrup B, Jenny T F, Jurczyk S C, Konig M, von Krosigk U, Lutz M J, MacPherson L J, Moroney S E, Muller E, Nambiar K P, Piccirilli J A, Switzer C Y, Vogel J J, Richert C, Roughton A L, Schmidt J, Schneider K C, Stackhouse J, Pure Appl. Chem. 1998, 70, 263-266.
[11] Hoshika S, Leal N A, Kim M J, Kim M S, Karalkar N B, Kim H J, Bates A M, Watkins N E, SantaLucia H A, Meyer A J, DasGupta S, Piccirilli J A, Ellington A D, SantaLucia J, Georgiadis M M, Benner S A, Science 2019, 363, 884-887.
[12] Disney M D, Dwyer B G, Childs-Disney J L, Cold Spring Harb. Perspect. Biol. 2018, 10, DOI 10.1101/cshperspect.a034769.
[13] Ursu A, Vezina-Dawod S, Disney M D, Drug Discov. Today 2019, 24, 2002-2016.
[14] Connelly C M, Boer R E, Moon M H, Gareiss P, Schneekloth J S Jr, ACS Chem. Biol. 2017, 12, 435-443.
[15] Abulwerdi F A, Xu W, Ageeli A A, Yonkunas M J, Arun G, Nam H, Schneekloth J S Jr, Dayie T K, Spector D, Baird N, Others, ACS Chem. Biol. 2019.
[16] Eubanks C S, Hargrove A E, Biochemistry 2019, 58, 199-213.
[17] Rzuczek S G, Colgan L A, Nakai Y, Cameron M D, Furling D, Yasuda R, Disney M D, Nat. Chem. Biol. 2017, 13, 188-193.
[18] Velagapudi S P, Cameron M D, Haga C L, Rosenberg L H, Lafitte M, Duckett D R, Phinney D G, Disney M D, Proc. Natl. Acad. Sci. U.S.A 2016, 113, 5898-5903.
[19] Barros S A, Chenoweth D M, Angew. Chem. Int. Ed Engl. 2014, 53, 13746-13750.
[20] Donlic A, Morgan B S, Xu J L, Liu A, Roble C Jr, Hargrove A E, Angew. Chem. Int. Ed Engl. 2018, 57, 13242-13247.
[21] Felsenstein K M, Saunders L B, Simmons J K, Leon E, Calabrese D R, Zhang S, Michalowski A, Gareiss P, Mock B A, Schneekloth J S Jr, ACS Chem. Biol. 2016, 11, 139-148.
[22] Disney M D, J. Am. Chem. Soc. 2019, 141, 6776-6790.
[23] Donlic A, Hargrove A E, Wiley Interdiscip. Rev. RNA 2018, 9, e1477.
[24] Connelly C M, Moon M H, Schneekloth J S Jr, Cell Chem Biol 2016, 23, 1077-1090.
[25] Morgan B S, Sanaba B G, Donlic A, Karloff D B, Forte J E, Zhang Y, Hargrove A E, ACS Chem. Biol. 2019, DOI 10.1021/acschembio.9b00631.
[26] Donlic A, Morgan B S, Xu J L, Liu A, Roble C Jr., Hargrove A E, Angew. Chem. Int. Ed Engl. 2018, 130, 13426-13431.
[27] Moon M H, Hilimire T A, Sanders A M, Schneekloth J S Jr, Biochemistry 2018, 57, 4638-4643.
[28] Warner K D, Homan P, Weeks K M, Smith A G, Abell C, Ferré-D'Amaré A R, Chem. Biol. 2014, 21, 591-595.
[29] Wang F, Fesik S W, Fragment-based Drug Discovery: Lessons and Outlook 2015.
[30] Yao R W, Wang Y, Chen L L, Nat. Cell Biol. 2019, 21, 542-551.
[31] Quinn J J, Chang H Y, Nat. Rev. Genet. 2016, 17, 47-62.
[32] Batista P J, Chang H Y, Cell 2013, 152, 1298-1307.
[33] Lange R F M, Beijer F H, Sijbesma R P, Hooft R W W, Kooijman H, Spek A L, Kroon J, Meijer E W, Angew. Chem. Int. Ed Engl. 1997, 36, 969-971.
[34] Zeng Y, Pratumyot Y, Piao X, Bong D, J. Am. Chem. Soc. 2012, 134, 832-835.
[35] Piao X, Xia X, Bong D, Biochemistry 2013, 52, 6313-6323.
[36] Liang Y, Miao S, Mao J, DeSantis C, Bong D, Biochemistry 2020, 59, 2410-2418.
[37] Liang Y, Mao J, Bong D, in Methods in Enzymology (Ed.: Hargrove A E), Academic Press, 2019, pp. 151-175.
[38] Mao J, DeSantis C, Bong D, J. Am. Chem. Soc. 2017, 139, 9815-9818.
[39] Mao J, Bong D, Synlett 2015, 26, 1581-1585.
[40] Xia X, Piao X, Bong D, J. Am. Chem. Soc. 2014, 136, 7265-7268.
[41] Piao X, Xia X, Mao J, Bong D, J. Am. Chem. Soc. 2015, 137, 3751-3754.
[42] Zhou Z, Xia X, Bong D, J. Am. Chem. Soc. 2015, 137, 8920-8923.
[43] Xia X, Zhou Z, DeSantis C, Rossi J J, Bong D, ACS Chem. Biol. 2019, 14, 1310-1318.
[44] Miao S, Liang Y, Marathe I, Mao J, DeSantis C, Bong D, J. Am. Chem. Soc. 2019, 141, 9365-9372.
[45] Xia X, Piao X, Fredrick K, Bong D, Chembiochem 2013, 15, 31-36.
[46] Li Q, Zhao J, Liu L, Jonchhe S, Rizzuto F J, Mandal S, He H, Wei S, Sleiman H F, Mao H, Mao C, Nat. Mater. 2020, 19, 1012-1018.
[47] Arambula J F, Ramisetty S R, Baranger A M, Zimmerman S C, Proc. Natl. Acad. Sci. U.S.A 2009, 106, 16068-16073.
[48] Fechter E J, Olenyuk B, Dervan P B, J. Am. Chem. Soc. 2005, 127, 16685-16691.
[49] Köhler O, Jarikote D V, Seitz O, Chembiochem 2005, 6, 69-77.
[50] Sato T, Sato Y, Nishizawa S, J. Am. Chem. Soc. 2016, 138, 9397-9400.
[51] Vilaivan T, Beilstein J Org. Chem. 2018, 14, 253-281.
[52] Chen M C, Cafferty B J, Mamajanov I, Gállego I, Khanam J, Krishnamurthy R, Hud N V, J. Am. Chem. Soc. 2014, 136, 5640-5646.
[53] Whitesides G M, Simanek E E, Mathias J P, Seto C T, Chin D, Mammen M, Gordon D M, Acc. Chem. Res. 1995, 28, 37-44.
[54] Ma M, Bong D, Langmuir 2011, 27, 8841-8853.
[55] Ma M, Bong D, Langmuir 2010, 27, 1480-1486.
[56] Vysabhattar R, Ganesh K N, Tetrahedron Lett. 2008, 49, 1314-1318.
[57] Branda N, Kurz G, Lehn J M, Chem. Commun. 1996, 2443.
[58] Chen D, Meena M, Sharma S K, MeLaughlin L W, J. Am. Chem. Soc. 2004, 126, 70-71.
[59] Chen H, Meena, Mclaughlin L W, J. Am. Chem. Soc. 2008, 130, 13190-13191.
[60] Kumar V, Gothelf K V, Org. Biomol. Chem. 2016, 14, 1540-1544.
[61] Schwergold C, Depecker G, Giorgio C D, Patino N, Jossinet F, Ehresmann B, Terreux R, Cabrol-Bass D, Condom R, Tetrahedron 2002, 58, 5675-5687.
[62] Mohapatra B, Pratibha, Saravanan R K, Verma S, Inorganica Chim. Acta 2019, 484, 167-173.
[63] Dolgosheina E V, Jeng S C Y, Panchapakesan S S S, Cojocaru R, Chen P S K, Wilson P D, Hawkins N, Wiggins P A, Unrau P J, ACS Chem. Biol. 2014, 9, 2412-2420.
[64] Kierzek R, Burkard M E, Turner D H, Biochemistry 1999, 38, 14214-14223.
[65] Mathews D H, Sabina J, Zuker M, Turner D H, J. Mol. Biol. 1999, 288, 911-940.
[66] Belousov S, Reuter J S, Seetin M G, Mathews D H, Nucleic Acids Res. 2013, 41, W471-4.
[67] Baeyens K J, De Bondt H L, Holbrook S R, Nat. Struct. Biol. 1995, 2, 56-62.
[68] Warner K D, Chen M C, Song W, Strack R L, Thom A, Jaffrey S R, Ferre-D'Amare A R, Nat. Struct. Mol. Biol. 2014, 21, 658-663.
[69] Huang H, Suslov N B, Li N S, Shelke S A, Evans M E, Koldobskaya Y, Rice P A, Piccirilli J A, Nat. Chem. Biol. 2014, 10, 686-691.
[70] Jang Y H, Hwang S, Chang S B, Ku J, Chung D S, J. Phys. Chem. A 2009, 113, 13036-13040.
[71] Geyer C R, Battersby T R, Benner S A, Structure 2003, 11, 1485-1498.

### Example 3. Non-Canonical Hybridization

### Overview

We have an ongoing program to expand the capability of bPNAs to triplex hybridize with noncanonical bulge sites in RNA (i.e., those which are not Watson-Crick basepaired). Using the synthetic base melamine, we have already rigorously established that a U-rich internal loop (URIL) comprised of 8 uridine bases in structured RNA (U4xU4) can be selectively targeted within the cell by bPNA probes. We have preliminary data that shows the ability of bPNAs that contain melamine and other bases to target URILs that are not completely uridine in content. In particular, bulge sites that contain UG or UC pairings may also be targeted using another synthetic base, ammeline, in conjunction with melamine. We also have found that a fully heterogeneous dyad (AG/CU) is a generally promiscuous site for targeting with synthetic pyrimidines and the native base cytidine. Our preliminary studies thus demonstrate that it is possible to expand the targeting capability of bPNAs beyond monotonic U-loops.

### Significance

A T/U targeting toehold for selective recognition of non-duplex domains within folded nucleic acids. Non-canonical interactions emerge at the interface domains between defined secondary structural elements, such as junctions, loops and bulges. These interface domains can be considered as structural transitions, with the potential to be stabilized by a recognition partner. In keeping with this notion, these sites of non-canonical interactions are often where functions such as molecular recognition (tertiary contacts) and catalysis emerge. To date, there is no general solution for the recognition for non-duplex regions within folded nucleic acids such as loop, junction or bulges; aminoglycosides have been found to bind proximal to these sites of duplex distortion, but promiscuously. We and others have demonstrated that melamine readily forms base-triples with two equivalents of thymine or uracil in the context of folded nucleic acids or when coupled to a nonspecific intercalator. While molecular recognition of uninterrupted oligo T/U loops is a solved problem using bPNAs, T/U-rich sequences punctuated with other nucleotides present a greater challenge. The larger arc of our research program considers the melamine base as a “toehold” in the molecular recognition of native T/U-rich non-duplex secondary structures to create a productive entry point for chemical probing and intervention in nucleic acid biology. The subset of T/U-rich non-duplex structures that are biologically important is considerable, including telomere TTA loops, trinucleotide repeat disorders (CUG/CTG repeats), and regulatory structures in the 3′UTR (AU-rich elements, UAU triple strands). If this recognition problem could be generally solved with synthetic reagents, the solution would yield a set of tools to annotate native macromolecular dynamics and interactions, discover new biological pathways and new therapeutic avenues. Our research efforts to develop engineered oligo-T/U domains as genetically encoded sites for probe and ligand installation are motivated by this larger molecular recognition problem, which we believe can be addressed by improving upon weak bPNA toehold binders to native T/U sites.

### Preliminary Data

Binding to heterogeneous URILs in RNA. We prepared RNA duplexes that hosted a 6×6 noncanonical bulge site flanked on both sides by 12 base pair helices (FIG. 22). This URIL contained variation (NNxNN) in the central tetrad. We synthesized bPNAs that were N-terminally labeled with carboxyfluorescein and contained three RNA-binding sites: K^{2M}K^{XX}K^{2M}, with beta alanine spacers in between each lysine derivative. We synthesized bPNA variants (Scheme 1) of the general form K^{2M}K^{XX}K^{2M}, where X is a lysine sidechain-displayed base (X=A, U, C, M, N) and the lysines are separated by beta alanine residues. Thus, we examined via native gel the binding of each bPNA to each tetrad variant by quantification of the Cbf stained RNA band on the gel. This gel data was normalized against a positive control (K^{2M}K^{2M}K^{2M} binding to U6xU6) and converted into a heat map of binding (FIG. 23). These data indicated that the central residue in bPNA could determine binding selectivity to the URIL variants, providing strong support to the notion that bPNA binding to heterogeneous URILs is possible by partial variation of base content in bPNA.

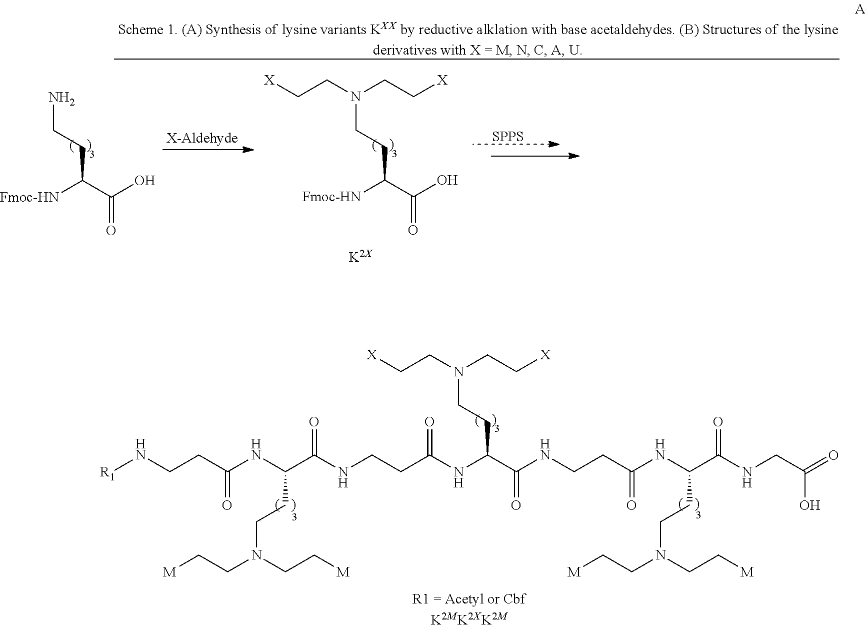

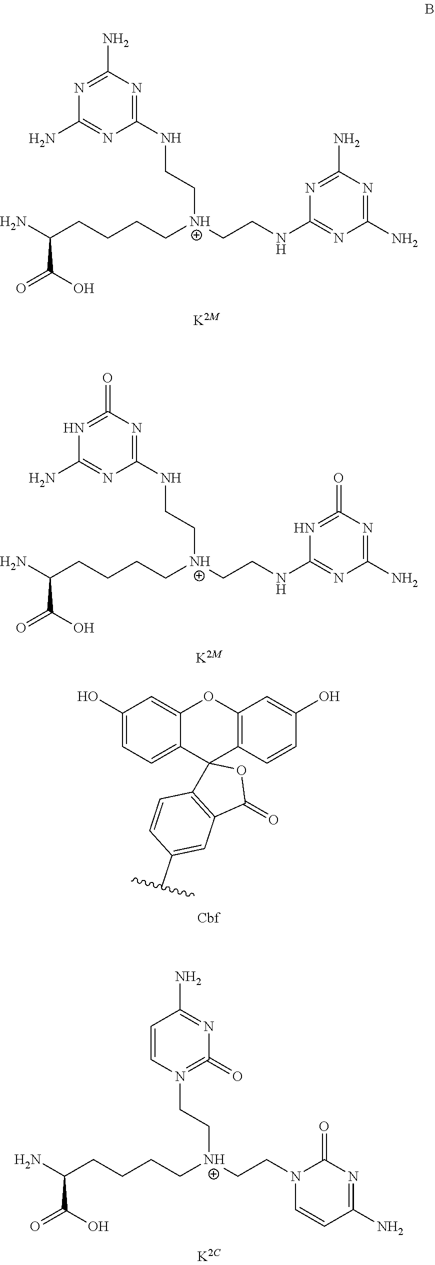

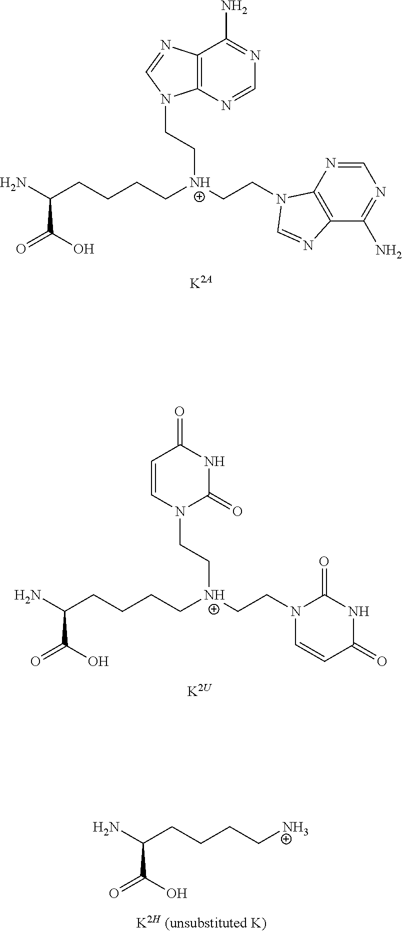 [SMILES: Nc1ncnc2c1ncn2CC[NH+](CCCC[C@H](N)C(=O)O)CCn1cnc2c(N)ncnc21 ; N[C@@H](CCCC[NH+](CCn1ccc(=O)[nH]c1=O)CCn1ccc(=O)[nH]c1=O)C(=O)O ; N[C@@H](CCCC[NH3+])C(=O)O]

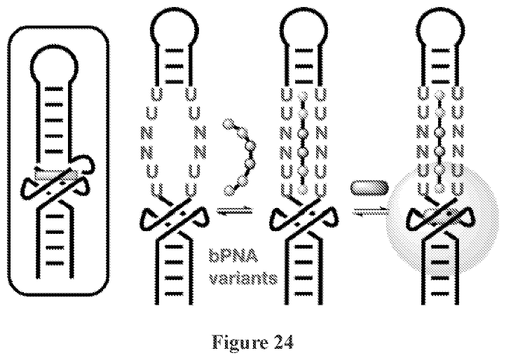

Fluorogenic turn-on with an RNA internal bulge cassette. We have developed a sensitive fluorescent readout for bPNA binding using a Spinach aptamer modified on P2 with a U₄XU₄ internal bulge. This fluorogenic RNA discriminates among unlabeled bPNA backbone variants in terms of DFHBI emission intensity (FIG. 24). The core concept of this assay is to connect stem restructuring via triplex hybridization to a fluorescence signal. This is accomplished by replacement of the P2 duplex stem with a URIL, which destructors the Spinach aptamer binding site, resulting in loss of fluorogenic dye-binding. We have previously demonstrated that bPNA binding to a URIL-spinach variant can restore fluorogenic binding. In this assay, we alter the 6×6 internal bulge of the URIL to contain a central tetrad (NNxNN) wherein N contains non-uridine bases. We find that these central tetrad variants are not activated by bPNAs containing only melamine (6 M bPNAs).

The N-acetylated bPNA derivatives K^{2M}K^{XX}K^{2M} were then used to bind to URIL variants of the spinach aptamer as described. Triplicated data confirmed the potential utility of the triazine base ammeline (N) to complement the binding of melamine to noncanonical sites. The data indicates fluorogenic turn-on caused by DFHBI dye binding, which in turn represents bPNA binding. As a positive control, we used a Spinach aptamer (the minimal baby spinach scaffold) without modification of P2. As indicated the variant is nearly off (inactive with regard to dye binding) in the absence of bPNA. Melamine-only bPNA (K^{2M}K^{2M}K^{2M}) largely restores fluorogenic dye binding but other derivatives also do.

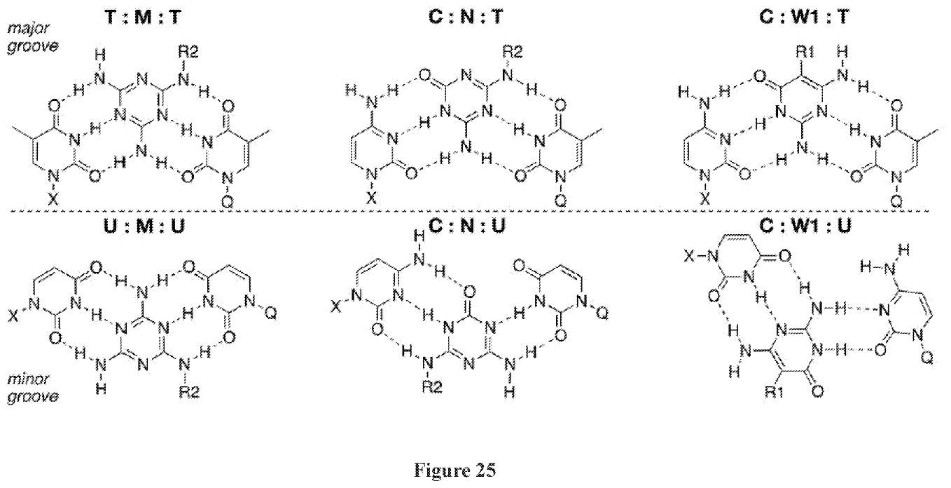

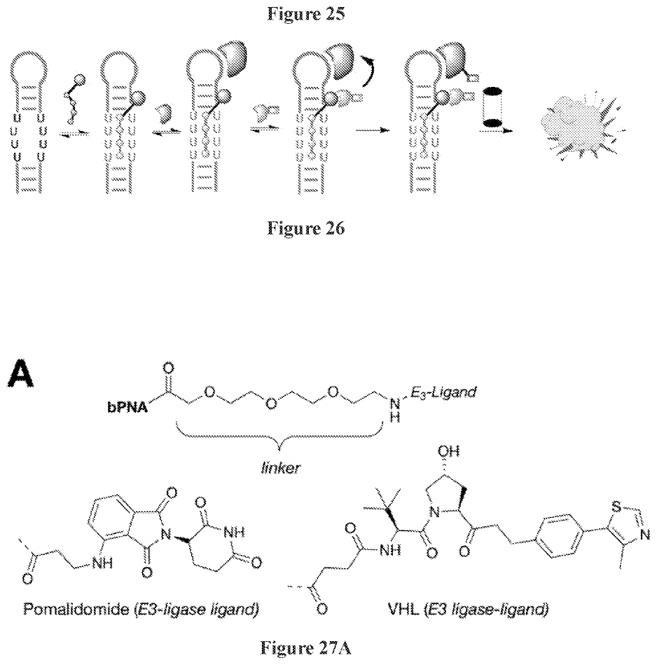

Notably, when the central tetrad in the URIL is altered to S′-GGxUU-3′ from all U, we observe preferential binding of the K^{2M}K^{2N}K^{2M} bPNA, which has two ammeline bases on the central residue rather than melamine. This selectivity is absent when the tetrad direction is reversed with 5′-UUxGG-3′, supportive of a selective binding interaction. Though no binding is seen for UUxCC, it is possible that binding may be observed for the reverse direction, CCxUU. Work is in progress to study other potential binding interactions such as the binding of ammeline (N) to CU or UC (FIG. 25).

### Example 4. RNA-PROTACs

### Overview

In this Example, we describe how bPNA-RNA hybrids can direct degradation of RNA binding proteins in ribonucleoprotein (RNP) complexes in a method we call RNA-PROTACs. This degradation strategy utilizes endogenous protein degradation pathways, similar to the method generally known as PROTACs. However, in the current method, the interactome surrounding a particular RNA secondary structure motif may be targeted for RNP degradation, allowing precise isolation of the PROTACs effect to the proteins that interact with a specific transcript site. This methodology has potential to result in development of novel therapeutics that act on dysregulated RNPs.

### Background and Significance

Ligand-directed proteolysis: RNA-PROTACs. Proteolysis targeting chimeras (PROTACs) are degradation strategies in which a small-molecule inhibitor is coupled to a ligand for E3-ubiquitin ligase. Small-molecule protein binding can direct ubiquitination of the target, marking the protein for proteolysis. Functionalization of bPNA with E3 ligase ligands and triplex hybridization to RNA can direct selective proteolysis of RBPs docked proximal to stem replacement modified sites in an approach we call RNA-PROTACs. Interestingly, lncRNA HOTAIR is known to direct RBP proteolysis through scaffolding of E3 ligase and substrates Ataxin-1 and Snurportin-1, resulting in ubiquitination and proteolysis of substrates, underscoring the biomimetic aspect of our approach. Unlike PROTACs methods that require conjugation to a transport domain, bPNA has established cell-penetrating properties. Of particular interest as substrates are prion-like proteins causative in neurodegenerative disorders (eg-ALS, Alzheimer's disease, Huntington's disease), which have few therapeutic levers. Prior methods have focused on binding Tau protein to drive ubiquitinylation; however, nearly all proteins that exhibit neurotoxic prion-like properties contain an RRM, and RNA-binding is essential for toxicity. Thus, an RNA-PROTACs approach enabled by bPNA is potentially a general platform for addressing the prion family of diseases. Importantly, the approach described herein enables precise knockdown of the RNP interactome centered at a specific RNA secondary structural motif. No other method can offer this selective outcome, and thus this strategy could hold a therapeutic advantage.

### Experimental Design

Initially, we focused on two well-studied ligands for Cereblon (CNBR) and Von Hippel Lindau (VHL) E3-ligases which have been shown to generate selective kinase degradation profiles in PROTACS, even when conjugated to promiscuous kinase inhibitor targeting modalities.

### Findings

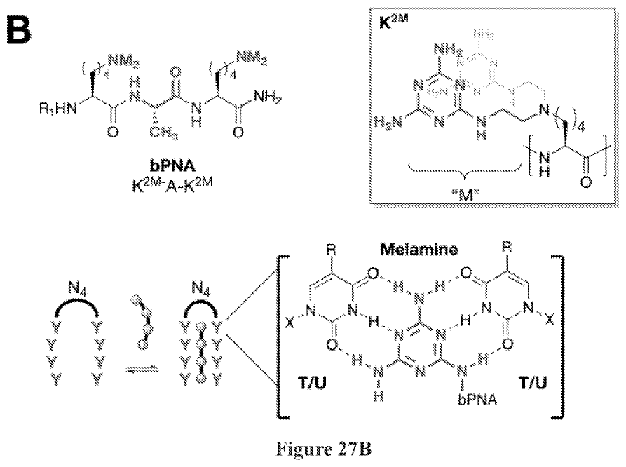

Following published synthetic protocols, we conjugated pomalidomide (POM, FIG. 27(A)), a CNBR ligand to the N-terminus of tripeptide bPNA (FIG. 27), using a linker verified to be effective in PROTACs.

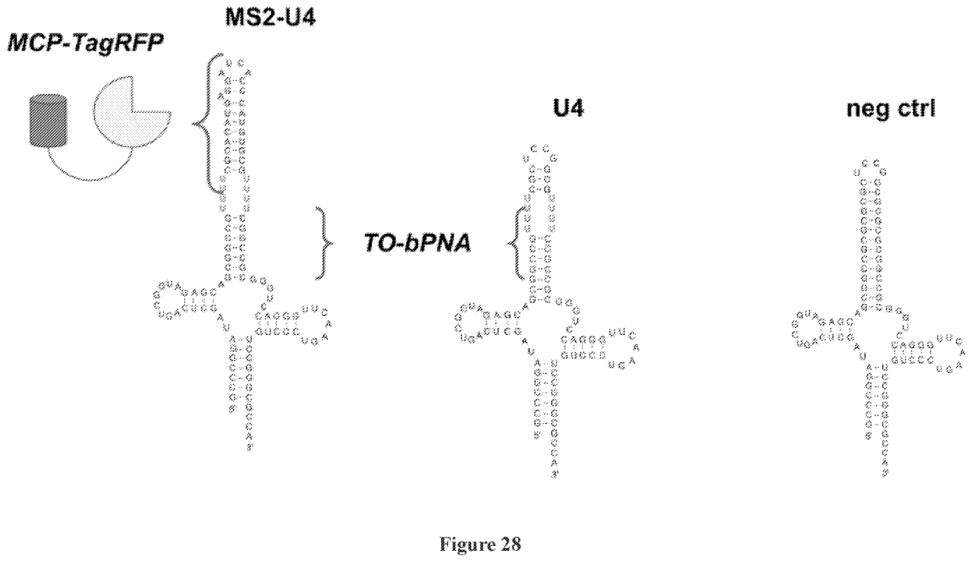

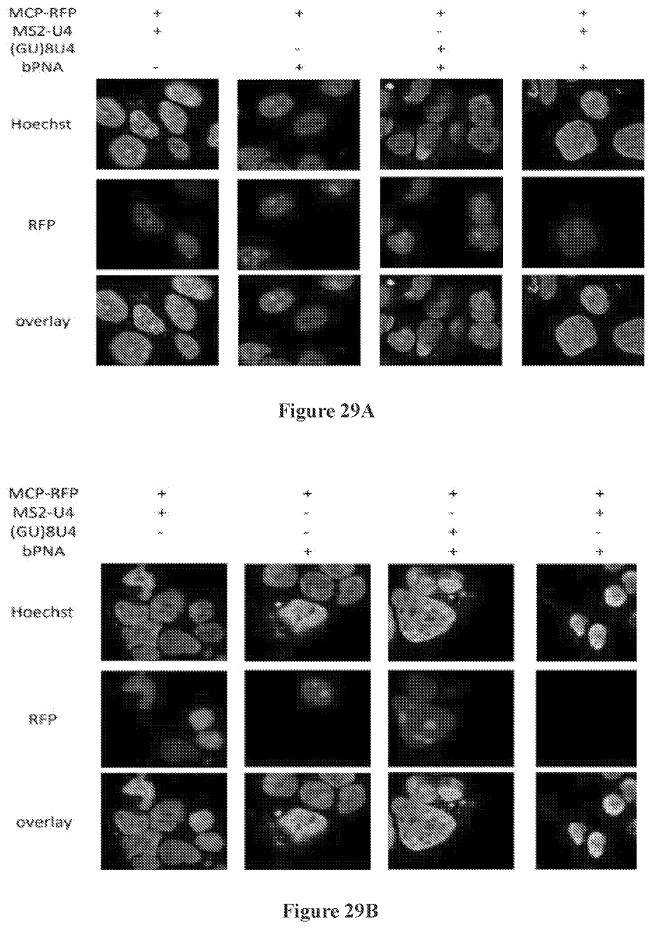

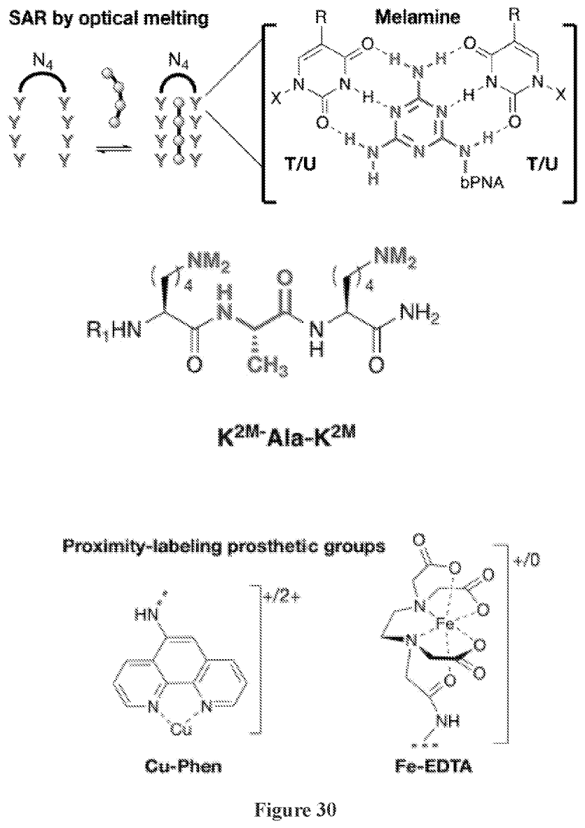

We then tested intracellular RNA-directed PROTACs using MS2 hairpin RNA, which pairs with MCP-RFP to form an RNP. We assessed RFP degradation by confocal fluorescence microscopy. An URIL binding site for bPNA (U4xU4) was inserted into a tRNA scaffold. This was used alone, or juxtaposed with an MS2 hairpin sequence. A negative control was also prepared that contained a base-paired region in place of the URIL (FIG. 28). Additionally, the structured hairpin in U4 tRNA was fused to an RNA (UG) 8 that does not bind MCP-RFP

These RNAs were introduced into HEK293 cells via plasmid transfection, along with a plasmid encoding MCP-tagRFP, which is nuclear-localized. Cells were treated in media with POM-bPNA at 0.5 micromolar concentration. Cells were harvested after 24 and 72 hr and demonstrated a marked loss of RFP signal. The RFP signal was not diminished when bPNA was left out or when the (GU) 8 U4 RNA was used in place of the MS2-U4 RNA. Thus, RNP formation is required to diminish the RFP signal, which is completely ablated in 72 hr. These data support intracellular degradation of the MCP-RFP protein via RNA-directed PROTACs.

### Example 5. bPNA-RNA Hybrids to Direct Proximity-Biotinylation Labeling of RNA Binding Proteins in Ribonucleoprotein (RNP) Complexes

### Overview

In this Example, we describe that bPNA-RNA hybrids can direct proximity biotinylation labeling of RNA binding proteins in Ribonucleoprotein (RNP) complexes. Proximity-labeling may be used to both verify suspected RNP complexes as well screen for interactions. While this concept is well-established for protein-centered proximity labeling, we disclose herein a method for RNA-centered proximity labeling with precision at the level of secondary structure motifs that is not possible with alternative technology. While there are reports of RNA or DNA directed proximity labeling, these prior methods are limited to specific nucleic acid sequences that must be introduced into the cell, or are precision-limited to the entire transcript and thus are not applicable to generally assess the global interactome centered at a particular structural motif. The method described herein is a broadly useful strategy for mapping out motif-specific interactomes within a transcript and thus will be highly impactful in discovery biology, including identification and verification of new drug targets.

### Background

Bifacial peptide nucleic acids (bPNAs) provide a general solution for labeling of U-rich internal loops (URILs) in RNA. Targeting or URILs with bPNAs offers a versatile platform for probing RNA biology via noncovalent modification with bPNAs bearing prosthetic groups, in particular groups useful for directing chemical labeling of RNA binding partners such as proteins within RNPs. Though chemical methods exist for modification, each method has drawbacks with regard to scope and yield and require specialized expertise. Crosslinking immunoprecipitation with high throughput sequencing (HITS-CLIP, PAR-CLIP) approaches eschew selective modification altogether to identify RNA binding partners by exploiting the ability of nucleobases to crosslink with binding partners upon exposure to UV light. Similarly, HTS chemical probing uses reagents with broad reactivity to modify exposed RNA sites in vivo to rapidly assess folded domains and potential sites of tertiary contact. Proximity-labeling refers to indiscriminate enzymatic modification of nearby biomolecules, typically with biotin; this enables spatially resolved proteomic or Seq-based census of intracellular compartments when organelle-anchored enzymes are used, or interaction partners of soluble fusion proteins. Though powerful, this centers proximity-labeling on the enzyme instead of the molecule of interest. Furthermore, site-specific studies are needed to elucidate the details of engagement beyond the global view provided by HTS methods. This next level of investigation can occur in vitro in fixed cells only (FISH) or live cells (molecular beacons, bacteriophage-derived tags, Cas-based targeting, fluorogenic aptamers). The application of transglucosylases can also covalently modify RNA, but appears to work best in vitro, unlike proximity-labeling which operates in live cells. The use of stem-replacement triplex hybridization with bPNA to install a prosthetic group allows us to direct URIL-proximity labeling of RNPs with motif-specific precision and without the use of pendant enzymes or limitations on RNA context. Our preliminary studies strongly suggest that triplex hybridization with bifacial peptide nucleic acids (bPNAs) can drive intracellular biotinylation of known RNPs. These data provide a strong scientific premise for the notion that bPNAs provide a platform for RNA directed proximity labeling that has significant advantages over known methods.

### Experimental Design

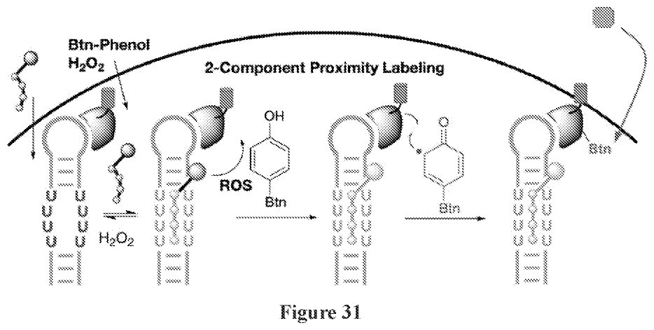

We synthesized bPNAs bearing ROS-generating metal centers (Fe-EDTA, Cu-Phen), which are known to direct oxidation of DNA and proteins via ROS generation of free hydroxyl radicals (Fe-EDTA) or metal-bound radical species (Cu-Phen). We previously demonstrated that bPNAs modified in this way could freely penetrate mammalian cells in culture, bind and cleave DNA targets, while RNA substrates were resistant to oxidative cleavage. The high reactivity of ROS generated from these metal centers is known to react with phenol and derivatives and was thus expected to react with the phenol group in biotin-phenol to generate a phenoxyl radical, as known for peroxidase based proximity labeling. We therefore expected that Fe-EDTA and Cu-Phen displaying bPNAs could generate ROS centered on its RNA substrate. These ROS would then generate a phenoxyl radical with biotin-phenol, identical to that generated by APEX proximity labeling (FIG. 31). The ROS and phenoxyl radicals generated in proximity of the bPNA-RNA hybridization site would then effect proximity biotinylation as previously reported, but centered on RNA secondary structural motifs (URIL) and without the use of a pendant or membrane-anchored peroxidase enzyme.

### Findings

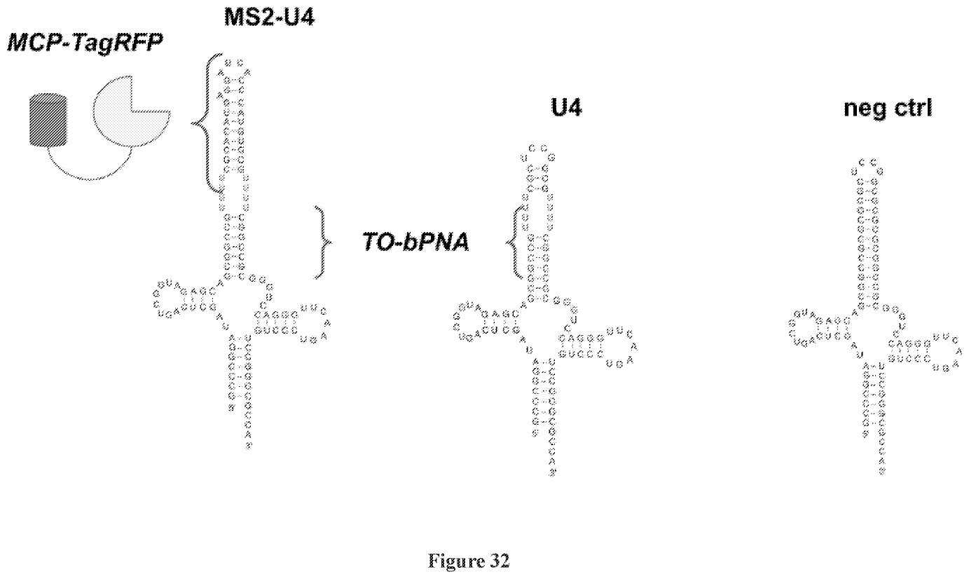

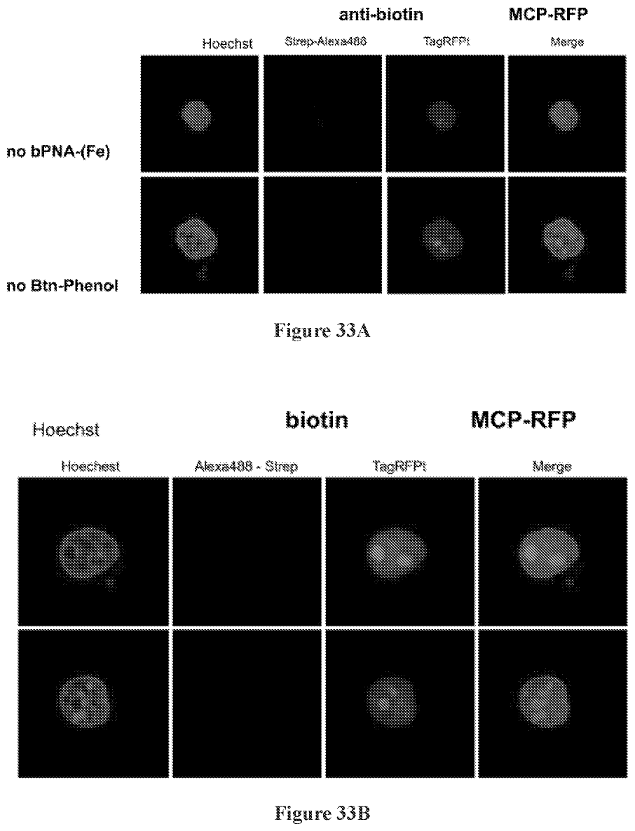

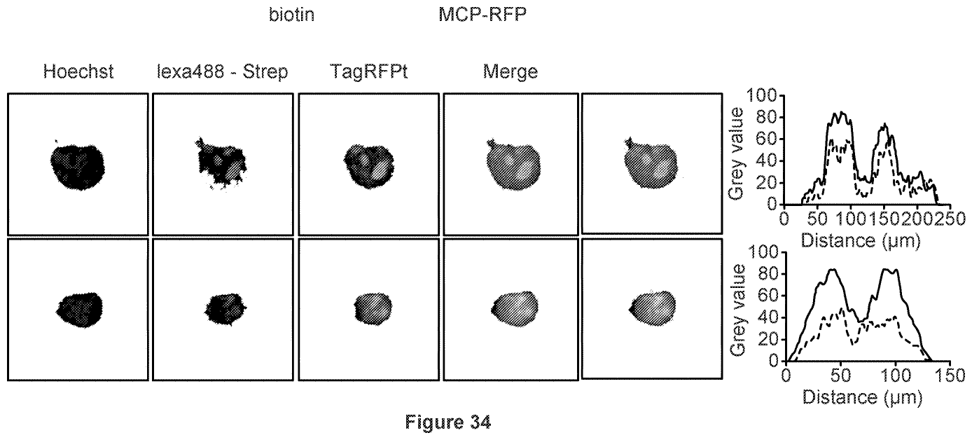

We tested intracellular RNA-directed biotinylation using MS2 hairpin/MCP protein pairs and assessed by intracellular streptavidin-488 co-labeling. An URIL binding site for bPNA (U4xU4) was inserted into a tRNA scaffold. This was used alone, or juxtaposed with an MS2 hairpin sequence. A negative control was also prepared that contained a base-paired region in place of the URIL (FIG. 32).

These RNAs were introduced into HEK293 cells via plasmid transfection, along with a plasmid encoding MCP-tagRFP, which is nuclear-localized. Cells were treated in media with 0.5 micromolar ROS-bPNA (Fe-EDTA) and 1 mM biotin-phenol. Unlike APEX proximity labeling, no additional oxidant was added. Addition of H₂O₂ was found to result in non-specific oxidation in our hands as well as the literature data. Following treatment, proximity labeling with biotin was assessed by fixing and permeabilizing cells, followed by staining with Streptavidin-488 (modified with Alexa-488). We judged co-localization of the green Alexa-488 signal with RFP to indicate successful biotinylation of the MCP-RFP fusion protein.

### Conclusions

ROS-bPNA can target URILs in endogenously expressed RNAs and effect proximity-biotinylation of RNPS in an intracellular context in mammalian cells. This method is convenient and avoids the use of cumbersome enzyme conjugation and enables the novel application of proximity labeling concepts to secondary structure motifs in RNA. There is no existing technology which can provide proximity labeling at the sub-transcript level of precision with such a general platform.

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

1. A compound defined by Formula I below 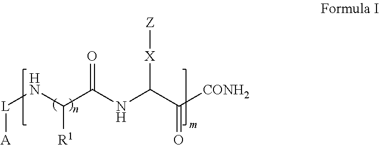
wherein
A represents a prosthetic group;
X is absent or represents a first bivalent linking group;
L is absent or represents a second bivalent linking group;
n is, individually for each occurrence, an integer selected from 1 and 2;
m is an integer selected from 2, 3, and 4;
Z represents, individually for each occurrence, a binding motif selected from one of the following 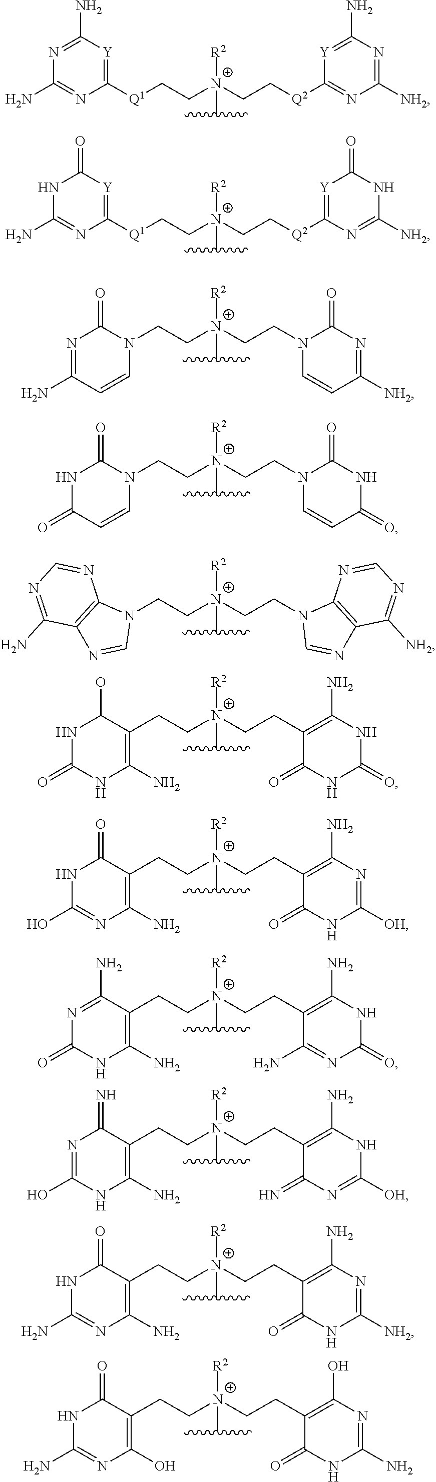
Q¹ and Q² individually represent —O— or —NR^{A}—;
Y is N, —CR^{B}—,
R¹ is, individually for each occurrence, selected from one of the following 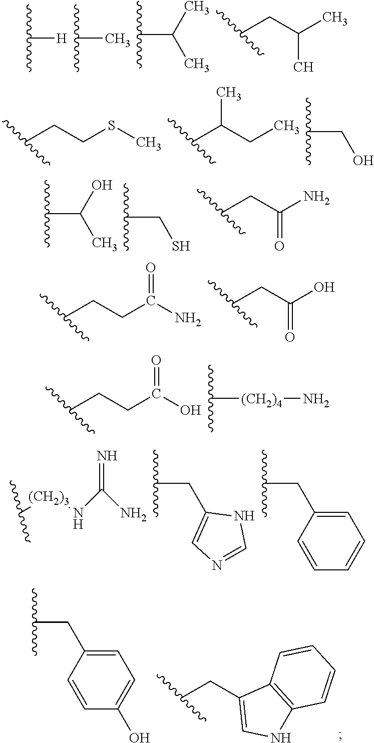
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

2. (canceled)

3. The compound of claim 1, wherein R² represents H for each occurrence.

4. (canceled)

5. The compound of claim 1, wherein Q¹ and Q² represent —NH— for each occurrence.

6. The compound of claim 1, wherein R¹ is, individually for each occurrence, selected from one of the following

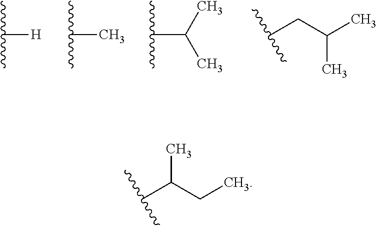

7. The compound of claim 1, wherein R¹ is, individually for each occurrence, —H or —CH₃.

8. (canceled)

9. (canceled)

10. (canceled)

11. (canceled)

12. The compound of claim 1, wherein X represents a first bivalent linking group and L represents a second bivalent linking group.

13. (canceled)

14. (canceled)

15. (canceled)

16. (canceled)

17. (canceled)

18. The compound of claim 1, wherein in at least one occurrence, Z represents the binding motif shown below 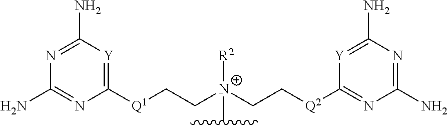
wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

19. The compound of claim 1, wherein m is 3 and Z represents the binding motif shown below in two occurrences 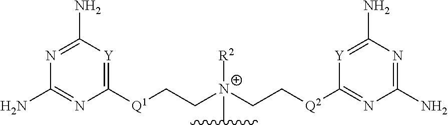
wherein
Q¹ and Q² individually represent —O— or —NR^{A}—;
Y is N, —CR^{B}—,
R² represents, individually for each occurrence, H, a C1-C4 alkyl group, a C2-C4 alkenyl group, a C2-C4 alkynyl group, or a C1-C4 haloalkyl group;
R^{A} represents, individually for each occurrence, H, a C1-C4 alkyl group, a C2-C4 alkenyl group, a C2-C4 alkynyl group, or a C1-C4 haloalkyl group; and
R^{B} represents, individually for each occurrence, H, halogen, methyl, or trifluoromethyl.

20. The compound of claim 1, wherein the compound is defined by Formula IA below 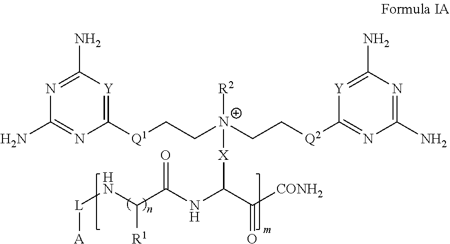
wherein A, L, Y, Q¹, Q², X, n, and m are as defined above

21. The compound of claim 1, wherein the compound is defined by Formula IA 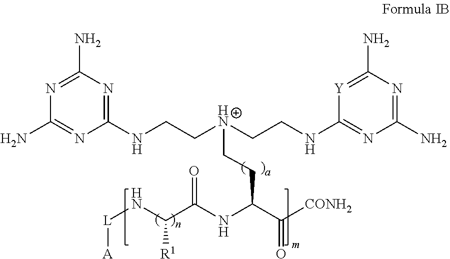
wherein
A, L, R¹, m, and n are as defined above; and
a is, individually for each occurrence, an integer selected from 3, 4, 5, and 6.

22. (canceled)

23. The compound of claim 1, wherein the prosthetic group is selected from the group consisting of a fluorogenic dye, a protein ligand, a redox-active center, an ROS-generating center, a photoreactive center, a spin label, a group transfer agent, a therapeutic agent, a catalytic center, a click motif, or an NMR active label.

24. The compound of claim 23, wherein the prosthetic group is a fluorogenic dye selected from the group consisting of thiazole derived dyes such as thiazole orange, dimethylindole red and derivatives thereof, symmetric cyanine dyes, asymmetric cyanine dyes, fluoresceins, rhodamines, fluorogenic variants of asymmetric cyanine dyes and fluoresceins such as JF635 and JF646, arsenate dyes such as FIASH and ReASH, malachite green and derivatives thereof, coumarin dyes, and hydroxybenzylidene dyes.

25. (canceled)

26. (canceled)

27. The compound of claim 23, wherein the prosthetic group is a protein ligand selected from the group consisting of a ubiquitin ligase ligand, a ligand for a translational activator, a ligand for a translational inhibitor, a ligand for a transcription activator, a ligand for a transcription inhibitor, a ligand for a nuclease, or a ligand for a cell surface protein.

28. (canceled)

29. The compound of claim 27, wherein the protein ligand is a ubiquitin ligase ligand that binds to an E3 ligase selected from the group consisting of XIAP, VHL, cereblon, and MDM2.

30. The compound of claim 1, wherein the compound binds to a non-canonical base pairing site in a nucleic acid via triplex hybridization.

31. (canceled)

32. The compound of claim 30, wherein the non-canonical base pairing site comprises a U-rich internal loop (URIL).

33. The compound of claim 32, wherein the URIL is a loop that includes at least four non-canonical base pairs, wherein at least 50% of the non-canonical base pairs comprise U—U pairs.

34. The compound of claim 33, wherein the URIL comprises from 4-8 non-canonical base pairs.

35. A method of detecting a target nucleic acid comprising
contacting the target nucleic acid with a bifacial peptide nucleic acid probe comprising a triplex hybrid forming moiety conjugated to a fluorogenic dye, wherein the bifacial peptide nucleic acid probe binds to a non-canonical base pairing site in the target nucleic acid via triplex hybridization.

36. (canceled)

37. A method of selectively degrading a target protein in vivo, the method comprising
contacting a nucleic acid with a bifacial peptide nucleic acid probe comprising a triplex hybrid forming moiety conjugated to a ubiquitin ligase ligand,
wherein the bifacial peptide nucleic acid probe binds to a non-canonical base pairing site in the target nucleic acid via triplex hybridization;
wherein the nucleic acid forms a ribonucleoprotein complex with the nucleic acid; and
wherein the ubiquitin ligase ligand of the bifacial peptide nucleic acid probe bound to the nucleic acid directs ubiquitination of the target protein, thereby directing proteolysis of the target protein.

38. (canceled)

39. A method of selectively labeling a target protein in vivo, the method comprising
contacting a nucleic acid with a bifacial peptide nucleic acid probe comprising a triplex hybrid forming moiety conjugated to a redox-active center, an ROS-generating center, a photoreactive center, or a catalytic center,
wherein the bifacial peptide nucleic acid probe binds to a non-canonical base pairing site in the target nucleic acid via triplex hybridization;
wherein the nucleic acid forms a ribonucleoprotein complex with the target protein; and
wherein the redox-active center, the ROS-generating center, the photoreactive center, or the catalytic center of the bifacial peptide nucleic acid probe bound to the nucleic acid participates in a chemical reaction that results in covalent modification of the target protein, thereby labeling the target protein.

40. (canceled)

41. (canceled)

42. (canceled)


SEQUENCE LISTING (60 sequences)

SEQ ID NO: 1 - RNA, 28 nt, synthetic construct
  misc_feature 1..28: note = Description of sequence: RNA duplex monomer
  source 1..28: mol_type = other RNA
cgcatagctcagttttgactcgatacgc

SEQ ID NO: 2 - RNA, 28 nt, synthetic construct
  misc_feature 1..28: note = Description of sequence: RNA duplex monomer
  source 1..28: mol_type = other RNA
gcgtatcgagtcttttctgagctatgcg

SEQ ID NO: 3 - RNA, 30 nt, synthetic construct
  misc_feature 1..30: note = Description of sequence: RNA duplex monomer
  source 1..30: mol_type = other RNA
cgcatagctcagttttttgactcgatacgc

SEQ ID NO: 4 - RNA, 30 nt, synthetic construct
  misc_feature 1..30: note = Description of sequence: RNA duplex monomer
  source 1..30: mol_type = other RNA
gcgtatcgagtcttttttctgagctatgcg

SEQ ID NO: 5 - RNA, 31 nt, synthetic construct
  misc_feature 1..31: note = Description of sequence: RNA duplex monomer
  source 1..31: mol_type = other RNA
ggcagcttttttttggtagttttttgctgcc

SEQ ID NO: 6 - RNA, 19 nt, synthetic construct
  misc_feature 1..19: note = Description of sequence: RNA duplex monomer
  source 1..19: mol_type = other RNA
gcaccgctaccaacggtgc

SEQ ID NO: 7 - RNA, 91 nt, synthetic construct
  misc_feature 1..91: note = Description of sequence: U4-tRNA
  source 1..91: mol_type = other RNA
gcccggatagctcagtcggtagagcagcggccgttttcgctccggcgttttcggccgcgggtccagggttcaagtccctgttcgggcgcca

SEQ ID NO: 8 - RNA, 106 nt, synthetic construct
  misc_feature 1..106: note = Description of sequence: U4-MS2 (MBSV5) tRNA
  source 1..106: mol_type = other RNA
gcccggatagctcagtcggtagagcagcggccgttttcgcacatgaggatcacccatgtgcgttttcggccgcgggtccagggttcaagtccctgttcgggcgcca

SEQ ID NO: 9 - RNA, 91 nt, synthetic construct
  misc_feature 1..91: note = Description of sequence: NEG tRNA
  source 1..91: mol_type = other RNA
gcccggatagctcagtcggtagagcagcggccgcgcgcgctccggcgcgcgcggccgcgggtccagggttcaagtccctgttcgggcgcca

SEQ ID NO: 10 - RNA, 46 nt, synthetic construct
  misc_feature 1..46: note = Description of sequence: U4-(GU)8 RNA
  source 1..46: mol_type = other RNA
cggccgttttcgctccggcgttttcggccggtgtgtgtgtgtgtgt

SEQ ID NO: 11 - DNA, 12 bp, synthetic construct
  misc_feature 1..12: note = Description of sequence: Target Sequence
  source 1..12: mol_type = other DNA
ggagaggctggg

SEQ ID NO: 12 - DNA, 12 bp, synthetic construct
  misc_feature 1..12: note = Description of sequence: Target Sequence
  source 1..12: mol_type = other DNA
agcagatgtagg

SEQ ID NO: 13 - DNA, 10 bp, synthetic construct
  misc_feature 1..10: note = Description of sequence: DNA duplex monomer
  source 1..10: mol_type = other DNA
ctggccgcgc

SEQ ID NO: 14 - DNA, 10 bp, synthetic construct
  misc_feature 1..10: note = Description of sequence: DNA duplex monomer
  source 1..10: mol_type = other DNA
ctggcggcgc

SEQ ID NO: 15 - DNA, 10 bp, synthetic construct
  misc_feature 1..10: note = Description of sequence: DNA duplex monomer
  source 1..10: mol_type = other DNA
ctggcagcgc

SEQ ID NO: 16 - DNA, 10 bp, synthetic construct
  misc_feature 1..10: note = Description of sequence: DNA duplex monomer
  source 1..10: mol_type = other DNA
ctggctgcgc

SEQ ID NO: 17 - DNA, 10 bp, synthetic construct
  misc_feature 1..10: note = Description of sequence: DNA duplex monomer
  source 1..10: mol_type = other DNA
gcgccgccag

SEQ ID NO: 18 - DNA, 10 bp, synthetic construct
  misc_feature 1..10: note = Description of sequence: DNA duplex monomer
  source 1..10: mol_type = other DNA
gcgcggccag

SEQ ID NO: 19 - DNA, 10 bp, synthetic construct
  misc_feature 1..10: note = Description of sequence: DNA duplex monomer
  source 1..10: mol_type = other DNA
gcgcagccag

SEQ ID NO: 20 - DNA, 10 bp, synthetic construct
  misc_feature 1..10: note = Description of sequence: DNA duplex monomer
  source 1..10: mol_type = other DNA
gcgctgccag

SEQ ID NO: 21 - RNA, 10 nt, synthetic construct
  misc_feature 1..10: note = Description of sequence: RNA duplex monomer
  source 1..10: mol_type = other RNA
ctggccgcgc

SEQ ID NO: 22 - RNA, 10 nt, synthetic construct
  misc_feature 1..10: note = Description of sequence: RNA duplex monomer
  source 1..10: mol_type = other RNA
ctggcggcgc

SEQ ID NO: 23 - RNA, 10 nt, synthetic construct
  misc_feature 1..10: note = Description of sequence: RNA duplex monomer
  source 1..10: mol_type = other RNA
ctggcagcgc

SEQ ID NO: 24 - RNA, 10 nt, synthetic construct
  misc_feature 1..10: note = Description of sequence: RNA duplex monomer
  source 1..10: mol_type = other RNA
ctggctgcgc

SEQ ID NO: 25 - RNA, 10 nt, synthetic construct
  misc_feature 1..10: note = Description of sequence: RNA duplex monomer
  source 1..10: mol_type = other RNA
gcgccgccag

SEQ ID NO: 26 - RNA, 10 nt, synthetic construct
  misc_feature 1..10: note = Description of sequence: RNA duplex monomer
  source 1..10: mol_type = other RNA
gcgcggccag

SEQ ID NO: 27 - RNA, 10 nt, synthetic construct
  misc_feature 1..10: note = Description of sequence: RNA duplex monomer
  source 1..10: mol_type = other RNA
gcgcagccag

SEQ ID NO: 28 - RNA, 10 nt, synthetic construct
  misc_feature 1..10: note = Description of sequence: RNA duplex monomer
  source 1..10: mol_type = other RNA
gcgctgccag

SEQ ID NO: 29 - skipped

SEQ ID NO: 30 - RNA, 10 nt, synthetic construct
  misc_feature 1..10: note = Description of sequence: RNA duplex monomer
  source 1..10: mol_type = other RNA
  misc_feature 5..6: note = Mismatch site
ctgggtgcgc

SEQ ID NO: 31 - RNA, 10 nt, synthetic construct
  misc_feature 1..10: note = Description of sequence: RNA duplex monomer
  source 1..10: mol_type = other RNA
  misc_feature 5..6: note = Mismatch site
ctggttgcgc

SEQ ID NO: 32 - RNA, 10 nt, synthetic construct
  misc_feature 1..10: note = Description of sequence: RNA duplex monomer
  source 1..10: mol_type = other RNA
  misc_feature 5..6: note = Mismatch site
ctggcagcgc

SEQ ID NO: 33 - RNA, 10 nt, synthetic construct
  misc_feature 1..10: note = Description of sequence: RNA duplex monomer
  source 1..10: mol_type = other RNA
  misc_feature 5..6: note = Mismatch site
ctggctgcgc

SEQ ID NO: 34 - RNA, 10 nt, synthetic construct
  misc_feature 1..10: note = Description of sequence: RNA duplex monomer
  source 1..10: mol_type = other RNA
  misc_feature 5..6: note = Mismatch site
ctggaagcgc

SEQ ID NO: 35 - RNA, 10 nt, synthetic construct
  misc_feature 1..10: note = Description of sequence: RNA duplex monomer
  source 1..10: mol_type = other RNA
  misc_feature 5..6: note = Mismatch site
ctggtagcgc

SEQ ID NO: 36 - RNA, 10 nt, synthetic construct
  misc_feature 1..10: note = Description of sequence: RNA duplex monomer
  source 1..10: mol_type = other RNA
  misc_feature 5..6: note = Mismatch site
ctggccgcgc

SEQ ID NO: 37 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcgtccag

SEQ ID NO: 38 - RNA, 10 nt, synthetic construct
  misc_feature 1..10: note = Description of sequence: RNA duplex monomer
  source 1..10: mol_type = other RNA
  misc_feature 5..6: note = Mismatch site
gcgctgccag

SEQ ID NO: 39 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcttccag

SEQ ID NO: 40 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcgaccag

SEQ ID NO: 41 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcctccag

SEQ ID NO: 42 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcgcccag

SEQ ID NO: 43 - RNA, 10 nt, synthetic construct
  misc_feature 1..10: note = Description of sequence: RNA duplex monomer
  source 1..10: mol_type = other RNA
  misc_feature 5..6: note = Mismatch site
gcgccaccag

SEQ ID NO: 44 - RNA, 10 nt, synthetic construct
  misc_feature 1..10: note = Description of sequence: RNA duplex monomer
  source 1..10: mol_type = other RNA
  misc_feature 5..6: note = Mismatch site
gcgctaccag

SEQ ID NO: 45 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcacccag

SEQ ID NO: 46 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcccccag

SEQ ID NO: 47 - RNA, 10 nt, synthetic construct
  misc_feature 1..10: note = Description of sequence: RNA duplex monomer
  source 1..10: mol_type = other RNA
  misc_feature 5..6: note = Mismatch site
gcgcaaccag

SEQ ID NO: 48 - RNA, 91 nt, synthetic construct; identical to SEQ ID NO: 9
  misc_feature 1..91: note = Description of sequence: NEG tRNA
  source 1..91: mol_type = other RNA

SEQ ID NO: 49 - RNA, 106 nt, synthetic construct; identical to SEQ ID NO: 8
  misc_feature 1..106: note = Description of sequence: MS2-U4 tRNA
  source 1..106: mol_type = other RNA

SEQ ID NO: 50 - DNA, 24 bp, synthetic construct
  misc_feature 1..24: note = Description of sequence: Internal control sequence
  source 1..24: mol_type = other DNA
tagtagcagatgtaggaggtagtg

SEQ ID NO: 51 - DNA, 24 bp, synthetic construct
  misc_feature 1..24: note = Description of sequence: Internal control sequence
  source 1..24: mol_type = other DNA
cactacctcctacatctgctacta

SEQ ID NO: 52 - RNA, 135 nt, synthetic construct
  misc_feature 1..135: note = Description of sequence: Internal control sequence
  source 1..135: mol_type = other RNA
agcagatgtagggtttgagagctaccccagcagagcatatgggctcgctggggtagcaagttcaaataaggctagtccgttatcaacttggagcttttatatggttttgctccaagtggcaccgagtcggtgctt

SEQ ID NO: 53 - RNA, 27 nt, synthetic construct
  misc_feature 1..27: note = Description of sequence: TDP-43 binding sequence
  source 1..27: mol_type = other RNA
ttttacatgaggatcacccatgttttt

SEQ ID NO: 54-57 - skipped (4 entries)

SEQ ID NO: 58 - RNA, 106 nt, synthetic construct
  misc_feature 1..106: note = Description of sequence: tRNA constructs for proximity labeling
  source 1..106: mol_type = other RNA
gcccggatagctcagtcggtagagcagcggccgttttcgcacatgaggatcacccatgtgcgttttcggccgcgggtccagggttcaagtccctgtccgggcgcca

SEQ ID NO: 59 - RNA, 91 nt, synthetic construct
  misc_feature 1..91: note = Description of sequence: tRNA constructs for proximity labeling
  source 1..91: mol_type = other RNA
gcccggatagctcagtcggtagagcagcggccgttttcgctccggcgttttcggccgcgggtccagggttcaagtccctgtccgggcgcca

SEQ ID NO: 60 - RNA, 91 nt, synthetic construct
  misc_feature 1..91: note = Description of sequence: tRNA constructs for proximity labeling
  source 1..91: mol_type = other RNA
gcccggatagctcagtcggtagagcagcggccgcgcgcgctccggcgcgcgcggccgcgggtccagggttcaagtccctgtccgggcgcca